(19) United States
(12) Patent Application Publication
(10) Pub. No.: US 2020/0246404 A1
(43) Pub. Date: Aug. 6, 2020

(54) FORMULATIONS

(71) Applicant: Molecular Infusions, LLC, Franklin, MA (US)

(72) Inventors: Tuna Yucel, Medford, MA (US); Scott S. Finnance, Providence, RI (US); Stephen E. Zale, Hopkinton, MA (US); Nicholas J. Boylan, Boylston, MA (US); William Stephen Faraci, Arlington, MA (US)

(21) Appl. No.: 16/789,869

(22) Filed: Feb. 13, 2020

Related U.S. Application Data
(63) Continuation of application No. PCT/US2018/045714, filed on Aug. 8, 2018.
Application No. PCT/US2018/045714 is a continuation-in-part of application No. PCT/US2018/018382, filed on Feb. 15, 2018.
(60) Provisional application No. 62/546,149, filed on Aug. 16, 2017.
(60) Provisional application No. 62/640,158, filed on Mar. 8, 2018.
(60) Provisional application No. 62/459,086, filed on Feb. 15, 2017.
(60) Provisional application No. 62/546,149, filed on Aug. 16, 2017.

(51) Int. Cl. A61K 36/185; A23L 33/105; A23L 2/56; A61K 47/26; A61K 47/10; A61K 31/05; A61K 31/352; A61K 9/48; A61K 9/00

(52) U.S. Cl. CPC A61K 36/185; A23L 33/105; A23L 2/56; A61K 47/26; A61K 45/06; A61K 31/05; A61K 31/352; A61K 9/48; A61K 9/0053; A61K 47/10

(57) ABSTRACT

The invention provides for compositions comprising one or more or at least two active ingredient(s), e.g., a cannabinoid, cannabinoid extract, terpene, or terpene extract. The invention includes cannabinoid formulations, including self-emulsifying formulations and micellar dispersions, as well as methods of making and using the same. Some formulations comprise a cannabinoid and surfactant. The formulations have improved dissolution, stability, absorption and/or oral bioavailability. Some of the formulations are rapid dispersing formulations.

### CROSS-REFERENCE TO RELATED APPLICATIONS

This application is a continuation of International Application No. PCT/US2018/045714, filed on Aug. 8, 2018, which claims the benefit of U.S. Provisional Application Nos. 62/546,149, filed Aug. 16, 2017 and 62/640,158, filed Mar. 8, 2018. International Application No. PCT/US2018/045714 is also a continuation-in-part of International Application No. PCT/US2018/018382, filed Feb. 15, 2018, which claims the benefit of U.S. Provisional Application Nos. 62/459,086, filed Feb. 15, 2017 and 62/546,149, filed Aug. 16, 2017. The entire teachings of the above applications are incorporated herein by reference.

### FIELD OF THE INVENTION

The present invention provides for compositions comprising one or more or at least two active ingredient(s), e.g., a cannabinoid, cannabinoid extract, terpene, or terpene extract. The invention further provides for compositions comprising a surfactant and/or co-solvent, as well as methods of making and using the same. The compositions include self-emulsifying formulations and formulations that form micelle solution/dispersions. The compositions of the present invention are suitable for oral administration. The compositions increase drug solubilization through colloidal or micellar dispersion. The compositions may reduce the time of onset, effect of food on absorption, and potentially lower hepatic first-pass metabolism of the cannabinoid, thereby improving bioavailability.

### BACKGROUND OF THE INVENTION

Cannabinoids are a class of active compounds derived from the Cannabis sativa, Cannabis indica, or Cannabis hybrid plant commonly known as marijuana. The most notable cannabinoid is the phytocannabinoid tetrahydrocannabinol (THC), the primary psychoactive compound in cannabis. Delta-9-tetrahydrocannabinol (Δ9-THC) and delta-8-tetrahydrocannabinol (Δ8-THC) mimic the actions of anandamide and 2-arachidonoylglycerol neurotransmitters produced naturally in the body. These cannabinoids produce the effects associated with cannabis by binding to the CB1 cannabinoid receptors in the brain.

Cannabidiol (CBD) is another major constituent of the cannabis plant. Other cannabinoids include Cannabigerol (CBG), Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM), Tetrahydrocannabinolic acid (THCA), cannabinol (CBN), and Cannabidiolic Acid (CBDA).

Synthetic Δ9-tetrahydrocannabinol (dronabinol) is marketed under the trade name MARINOL®. Dronabinol is approved by the Food and Drug Administration (FDA) for the control of nausea and vomiting associated with chemotherapy and for appetite stimulation of AIDS patients suffering from the wasting syndrome. MARINOL® is a formulation of dronabinol in sesame oil presented as a soft gelatin capsule for oral administration. After oral administration, dronabinol has an onset of action of approximately 0.5 to 1 hours and peak effect at 2 to 4 hours. Duration of action for psychoactive effects is 4 to 6 hours, but the appetite stimulant effect of dronabinol may continue for 24 hours or longer after administration. Dronabinol is almost completely absorbed (90 to 95%) after single oral doses. Due to the combined effects of first pass hepatic metabolism and high lipid solubility, only 10 to 20% of the administered dose reaches the systemic circulation.

There is a need for additional, preferably less complex, self-emulsifying and micellar dispersion forming formulations, particularly those that are more stable, faster acting (i.e., have a faster onset of action), avoid or reduce hepatic first-pass metabolism, deliver more of the active ingredient(s) to the lymphatic system, or increase oral bioavailability for treating a variety of conditions. There also remains a need for more effective compositions for treating pain, inflammation and inflammatory diseases, including autoimmune inflammatory diseases such as MS, and other diseases, conditions, and pathologies. There is still a further need for composition comprising a lipophilic agent that disperses rapidly in aqueous medium and forms a transparent emulsion or micellar dispersion. The present invention addresses these needs by providing improved formulations, including rapid dispersing formulations, for use in a variety of conditions including pain, inflammation and inflammatory diseases, including autoimmune inflammatory diseases such as MS, nausea and vomiting, and other diseases or conditions.

### SUMMARY OF THE INVENTION

A first aspect provides a composition comprising:

at least one active ingredient.

In one embodiment, the composition comprises at least two active ingredients.

In one embodiment, at least one of the active ingredients is a cannabis extract.

In one embodiment, at least one of the active ingredients is selected from a cannabinoid, cannabinoid extract, terpene, or terpene extract.

In another embodiment, the composition comprises at least two active ingredients selected from: one or more cannabinoids and/or one or more terpenes and/or one or more other active ingredients selected from ethyl pyruvate, melatonin, caffeine or resveratrol.

In one embodiment, the composition comprises at least one active ingredient; and at least one surfactant.

In one embodiment, at least one of the active ingredients is a cannabis extract.

In one embodiment, the active ingredient is selected from a cannabinoid, cannabinoid extract, terpene, or terpene extract. In one embodiment, the composition comprises more than one active ingredient and/or more than surfactant.

In another embodiment, the composition further comprises a co-solvent.

In one embodiment, the composition comprises:

at least one active ingredient;

at least one surfactant; and

at least one fatty acid, at least one monoglyceride, at least one diglyceride, or at least one triglyceride, or a combination thereof.

In one embodiment, at least one of the active ingredients is a cannabis extract. In another embodiment, the active ingredient is selected from a cannabinoid, cannabinoid extract, terpene, or terpene extract. In a further embodiment, the composition comprises more than one active ingredient. In another embodiment, the composition comprises more than one surfactant. In another embodiment, the composition comprises more than one fatty acid, more than one monoglyceride, more than one diglyceride, more than one triglyceride, or a combination thereof. In another embodiment, the composition further comprises a co-solvent.

In another embodiment, the composition is a non-aqueous formulation. In another embodiment, the composition is a pharmaceutical composition, preferably an oral dosage form. In various embodiments, the oral dosage form is a solid, liquid, or semi-solid oral dosage form. In another embodiment, the invention provides for a unit dose of the composition.

A second aspect provides a method of making the composition of the first aspect or otherwise of the present invention, comprising the steps of:

providing at least one active ingredient,

providing at least one surfactant,

optionally, providing at least one co-solvent, and,

optionally, providing at least one fatty acid, at least one monoglyceride, at least one diglyceride, or at least one triglyceride, or a combination thereof;

combining said active ingredient, said surfactant and, optionally, said co-solvent and/or a fatty acid, monoglyceride, diglyceride, triglyceride, or a combination thereof to form a homogeneous or isotropic mixture.

In one embodiment, the method comprises providing more than one active ingredient. In another embodiment, the method comprises providing more than one surfactant. In another embodiment, the method comprises providing more than one fatty acid, more than one monoglyceride, more than one diglyceride, and/or more than one triglyceride. In another embodiment, the method comprises providing a co-solvent.

In one embodiment, at least one of the active ingredients is a cannabis extract.

In one embodiment, the active ingredient is selected from a cannabinoid, cannabinoid extract, terpene, or terpene extract.

A third aspect provides for a method of treating or preventing a disease or condition in a subject, e.g., human, including pain, nausea, and/or vomiting, autoimmune inflammatory diseases such as MS, and other diseases and conditions, comprising the step of administering to said subject an effective amount of a composition of the first aspect.

In one embodiment, the composition is a non-aqueous composition. In another embodiment, the composition is free of fats or oils. In another embodiment, the composition is free of exogenously added fatty acids, monoglycerides, diglycerides, or triglycerides (e.g., MCT or LCT). In another embodiment, the composition is a pharmaceutical composition. In another embodiment, the pharmaceutical composition comprises a physiologically/pharmaceutically acceptable excipient. In a further embodiment, the composition is a rapid dispersing formulation, forming a transparent emulsion or micellar dispersion within a time selected from: 5, 10, 15, 20, 25, 30, 35, 40, 45, 50, 55 or 60, or 90 seconds, or within 2, 2.5, 3, 3.5, 4, 4.5, or 5 minutes after addition to an aqueous medium at a final concentration of 0.1 wt % (composition) and at a temperature selected from: 4 degrees, 20 degrees, 40 degrees, or 60 degrees C. In the further embodiment, the rapid dispersing formulation does not require agitation (e.g., shaking or stirring) for emulsification/dispersion. In a further embodiment, the composition is a unit dose. In a further embodiment, the composition is an oral dosage form, or more preferably, a solid, liquid, or semi-solid, non-aqueous, oral dosage form.

### BRIEF DESCRIPTION OF THE FIGURES

FIG. 1. Emulsion particle size as a function of HLB number. Formulation surfactant content of 50 vol. % and aqueous emulsion concentration of 1.0 vol. %. Open and solid circles denote Polysorbate—Span 80 blends and pure polysorbates, respectively.

FIG. 2. Emulsion particle size as a function of HLB number at an aqueous emulsion concentration of 1.0 vol. %. Formulation surfactant content for squares, triangles and x symbols were 50, 75 and 90 vol. %, respectively.

FIG. 3. Particle size vs. turbidity rank for 1.0 vol. % emulsions.

FIG. 4. Emulsion particle size as a function of HLB number at an aqueous emulsion concentration of 0.1 vol. %. Formulation surfactant content for squares, triangles and x symbols were 50, 75 and 90 vol. %, respectively.

FIG. 5. Particle size vs. turbidity rank for 0.1 vol. % emulsions.

FIG. 6. Dilutability as a function of HLB number at an aqueous emulsion concentration of 1.0 vol. %. Formulation surfactant content for squares, triangles and x symbols were 50, 75 and 90 vol. %, respectively.

FIG. 7. Dissolution time and viscosity as a function of polysorbate 80 concentration in binary ethanol: polysorbate 80 system.

FIG. 8. Dissolution time and viscosity as a function of polyethylene glycol (PEG) 40 hydrogenated castor oil concentration in binary ethanol: polyethylene glycol (PEG) 40 hydrogenated castor oil system.

FIG. 9. Minimum mass ratio of PEG-40 hydrogenated castor oil to THC-distillate as a function of artificial orange flavor concentration in some beverage additive compositions that rapidly self-emulsify into micellar dispersions when added to water or another beverage of choice.

### DETAILED DESCRIPTION OF THE INVENTION

The term “comprising” is as used herein, is synonymous with “including,” “containing,” or “characterized by,” is inclusive or open-ended and does not exclude additional, unrecited elements or method steps, unless the context clearly requires otherwise. For example, a composition of the present invention “comprising an active ingredient” contains one or any number of active ingredients, unless otherwise specified.

The phrases “consists of” or “consisting of” are closed-ended and includes only those features specified. When used in a clause, the phrases “consists of” or “consisting of” limit only the element set forth in that clause.

The phrases “consists essentially of” and “consisting essentially of” are partially open and limited to features that do not materially affect the basic and novel characteristic(s)” of the claimed invention. For example, the phrases include an unrecited level of impurities that do not materially affect the basic and novel characteristic(s), e.g., activity or stability, of a composition of the invention.

As used herein, when a range is set forth as “between” two values, it is understood that the range is inclusive of the end values.

As used herein, the terms “treat”, “treating” or “treatment” means to alleviate, reduce or abrogate one or more symptoms or characteristics of a disease or disorder and may be curative, palliative, prophylactic or slow the progression of the disease or disorder.

The term “effective amount” means an amount of active ingredient(s) that will result in a desired effect or result. The term “therapeutically effective amount” means an amount of active ingredient(s) that will elicit a desired biological or pharmacological response, e.g., effective to prevent, alleviate, or ameliorate symptoms of a disease or disorder; slow, halt or reverse an underlying process or progression of a disease or disorder; partially or fully restore cellular function; or prolong the survival of the subject being treated.

The term “patient” or “subject” means an animal, including mammals, non-human animals, and especially humans. In one embodiment, the patient or subject is a human. In another embodiment, the patient or subject is a human male. In another embodiment, the patient or subject is a human female.

The present invention relates to compositions comprising a at least one active ingredient. In some embodiments, the composition comprises at least two active ingredients. In one embodiment, at least one of the active ingredients is a cannabinoid, cannabinoid extract, or cannabis extract. In some embodiments, the composition further comprises a surfactant. In some embodiments, the compositions further comprise a co-solvent. In some embodiments, the composition is a rapid dispersion formulation.

The compositions include self-emulsifying compositions, e.g., self-emulsifying drug delivery systems (SEDDS), oil-free, and micellar dispersions, comprising an active ingredient, e.g., cannabinoid. Some of the compositions comprise at least one co-solvent. Some of the compositions comprise at least one fatty acid, at least one monoglyceride, at least one diglyceride, at least one triglyceride, or a combination thereof. The compositions that comprise a triglyceride include compositions that comprise a medium chain triglyceride (MCT) or a long chain triglyceride (LCT), wherein the MCT and LCT, unless otherwise specified, represents either a single species of medium chain triglyceride or long chain triglyceride, respectively, or a mixture of medium chain triglycerides and/or long chain triglycerides. In the presence of an aqueous solvent some of the compositions produce emulsions via self-emulsifying mechanisms. The compositions, including self-emulsifying drug delivery systems (SEDDS) and micelles, (e.g., swollen micellar dispersions), of the present invention enhance oral bioavailability by the formation of colloidal dispersions, thus increasing solubility of the active ingredient.

In one embodiment, the composition is a non-aqueous composition. In another embodiment, the composition is free of fats or oils. In another embodiment, the composition is free of exogenously added fatty acids, monoglycerides, diglycerides, or triglycerides (e.g., MCT or LCT).

In one embodiment, the composition has a viscosity of less than or equal to: 100, 90, 80, 70, 60, 50, 45, 40, 35, 30, 25, 20, 17.5, 15, 12.5, 10, 9, 8, 7, 6, 5, 4, 3, 2, 1.5 cP at 20 degrees C.

In a further embodiment, the composition is a rapid dispersing formulation, forming a transparent emulsion or micellar dispersion within a time selected from: 5, 10, 15, 20, 25, 30, 35, 40, 45, 50, 55 or 60, or 90 seconds, or within 2, 2.5, 3, 3.5, 4, 4.5, or 5 minutes after addition to an aqueous medium, e.g., water or deionized water, at a temperature selected from: 4 degrees, 20 degrees, 40 degrees, or 60 degrees C., wherein the final concentration of the composition in the aqueous medium is 0.1 wt %. In the further embodiment, the rapid dispersing formulation does not require agitation (e.g., shaking or stirring) for emulsification/dispersion. In a further embodiment, the composition is a unit dose. In a further embodiment, the composition is an oral dosage form, or more preferably, a solid, liquid, or semi-solid, non-aqueous, oral dosage form.

In another embodiment, the composition is a pharmaceutical composition. In another embodiment, the pharmaceutical composition comprises a physiologically/pharmaceutically acceptable excipient.

The compositions of the present invention include formulations that avoid hepatic first-pass metabolism, in part, by targeting chylomicron/lipoprotein delivery. The compositions of the present invention include formulations that have a faster onset of action—the time it takes an active ingredient to reach a minimum effective concentration after the active ingredient is administered. The compositions of the present invention include formulations that have greater stability, greater oral bioavailability, or reduced individual variability of bioavailability, e.g., by reducing food-effect, greater efficacy, or, in the case of THC, a more intense psychotropic effect as compared to MARINOL® and may be formulated for immediate release.

The compositions of the present invention comprise at least one active ingredient. In one embodiment, at least one of the active ingredients is a cannabis extract. In another embodiment, at least one of the active ingredients is selected from a cannabinoid, cannabinoid extract, terpene, or terpene extract.

In some embodiments, the composition comprises at least two active ingredients. In one embodiment, at least one of the active ingredients is selected from one or more cannabis extract, cannabinoid, cannabinoid extract, terpene, or terpene extract, or combinations thereof; and at least one of the active ingredients is selected from one or more anti-insomnia, anti-tussive, opioid analgesic, decongestant, non-opioid analgesic/anti-inflammatory drug, anti-migraine drug, anti-emetic, anti-histamine, proton pump inhibitor, H2 antagonist/H2 blocker, tranquilizer, anticonvulsant, hypnotic, muscle relaxant, anti-psychotic, anti-diarrheal, Attention Deficit and Hyperactivity Disorder (ADHD) drug, anti-Parkinson disease drug, benzodiazepine, benzodiazepine antagonist, barbiturate, barbiturate antagonist, stimulant, stimulant antagonist, antidepressant, nutraceutical, nicotine, BCS Class II active ingredient, BCS Class IV active ingredient, an anti-multiple sclerosis (MS) drug, ethyl pyruvate, or a combination thereof.

In some embodiments, the composition comprises:

at least one active ingredient; and

a surfactant.

In one embodiment, the active ingredient(s) comprise an anti-insomnia. In further embodiments, the anti-insomnia is selected from any one or more of: melatonin, L-Theanine, trazodone, zolpidem, temazepam, elprazolam, amitriptyline, halcion, lorazepam, clonazepam, Intermezzo, eszopiclone, diphenhydramine, doxepin, mirtazapine, gabapentin, doxylamine, quetiapine, flurazepam, estazolam, olanzapine, Seconal, triazolam, zaleplon, secobarbital, chloral hydrate, oxazepam, quazepam, ramelteon, suvorexant, butabarbital, pentobarbital, phenobarbital, phenyltoloxamine, amobarbital, diphenhydramine, dimenhydrinate, diphenhydramine/magnesium salicylate, diphenhydramine/naproxen, diphenhydramine/aspirin, diphenhydramine/paracetamol, diphenhydramine/ibuprofen, or tasimelteon.

In one embodiment, the active ingredient(s) comprise an anti-tussive. In further embodiments, the anti-tussive is selected from any one or more of: benzonatate, caramiphen edisylate, chlophedianol, codeine, dextromethorphan hydrobromide, hydrocodone, levopropoxyphene, morphine, codeine, ethylmorphine, dihydrocodeine, benzylmorphine, laudanum, dihydroisocodeine, nicocodeine, nicodicodeine, hydrocodone, hydromorphone, acetyldihydrocodeine, thebacon, diamorphine (heroin), acetylmorphone, noscapine, or pholcodine.

In one embodiment, the active ingredient(s) comprise an opioid analgesic. In further embodiments, the opioid analgesic is selected from any one or more of: alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, or tramadol.

In one embodiment, the active ingredient(s) comprise a decongestant. In further embodiments, the decongestant is selected from any one or more of: pseudoephedrine hydrochloride, phenylephrine bitartrate, phenylephrine hydrochloride or pseudoephedrine sulfate.

In one embodiment, the active ingredient(s) comprise a non-opioid analgesic/anti-inflammatory drug. In further embodiments, the non-opioid analgesic/anti-inflammatory is selected from any one or more of: acetaminophen or a non-steroidal anti-inflammatory agent selected from the group consisting of aspirin, celecoxib, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, or isoxicam.

In one embodiment, the active ingredient(s) comprise an anti-migraine drug. In further embodiments, the anti-migraine drug is selected from any one or more of: 2-bromo-LSD, acetaminophen/dichloralphenazone/isometheptene mucate, almotriptin, alniditan, amidrine, avitriptan, caffeine/ergotamine, calcitonin gene-related peptide receptor antagonist, clonidine, dasolampanel, dihydroergotamine, dimetotiazine, donitriptan, dotarizine, eletriptan, ergotamine, ergotamine/chlorcyclizine/caffeine, flumedroxone acetate, iprazochrome, lasmiditan, lisuride, lomerizine, methysergide, migraleve, naratriptan, naproxen, naproxen/sumatripta, olcegepant, oxetorone, paracetamol/metoclopramide, prochlorperazine, promethazine, proxibarbital, rimegepant, rizatriptan, selurampanel, sumatriptan, telcagepant, tezampanel, topiramate, or zolmitriptan.

In one embodiment, the active ingredient(s) comprise an anti-emetic. In further embodiments, the anti-emetic is selected from any one or more of: dolasetron, granisetron, ondansetron, tropisetron, palonosetron, mirtazapine, metoclopramide, cyclizine, diphenhydramine, dimenhydrinate, meclizine, promethazine, or hydroxyzine.

In one embodiment, the active ingredient(s) comprise an anti-histamine. In further embodiments, the anti-histamine is selected from any one or more of: diphenhydramine, loratadine, desloratadine, meclizine, fexofenadine, pheniramine, cetirizine, promethazine, brompheniramine, clemastine fumarate or chlorpheniramine.

In one embodiment, the active ingredient(s) comprise a proton pump inhibitors (PPI). In further embodiments, the PPI is selected from any one or more of: omeprazole, esomeprazole, pantoprazole, lansoprazole, or rabeprazole.

In one embodiment, the active ingredient(s) comprise a H2 antagonist/H2 blocker. In further embodiments, the H2 antagonist/H2 blocker is selected from any one or more of: cimetidine, ranitidine, or famotidine, nizatidine, ebrotidine, mifentidine, roxatidine, pisatidine, or aceroxatidine.

In one embodiment, the active ingredient(s) comprise a tranquilizer. In further embodiments, the tranquilizer is selected from any one or more of: amobarbital, pentobarbital, secobarbital, phenobarbital, clonazepam, diazepam, estazolam, flunitrazepam, lorazepam, midazolam, nitrazepam, oxazepam, triazolam, temazepam, chlordiazepoxide, or alprazolam.

In one embodiment, the active ingredient(s) comprise an anticonvulsant. In further embodiments, the anti-convulsant is selected from any one or more of: elbamate, carbamazepine, oxcarbazepine, vigabatrin, progabide, tiagabine, topiramate, gabapentin, pregabalin, ethotoin, phenytoin, valproic acid, or lamotrigine.

In one embodiment, the active ingredient(s) comprise a hypnotic. In further embodiments, the hypnotic is selected from any one or more of: zolpidem, zaleplon, zopiclone, or eszopiclone.

In one embodiment, the active ingredient(s) comprise a muscle relaxant. In further embodiments, the muscle relaxant is selected from any one or more of: methocarbamol, carisoprodol, chlorzoxazone, cyclobenzaprine, gabapentin, metaxalone, or orphenadrine.

In one embodiment, the active ingredient(s) comprise an anti-psychotic. In further embodiments, the anti-psychotic is selected from any one or more of: haloperidol, droperidol, chlorpromazine, fluphenazine, perphenazine, prochlorperazine, thioridazine, trifluoperazine, mesoridazine, promazine, triflupromazine, levomepromazine, methotrimeprazine, pimozide, chlorprothixene, flupenthixol, thiothixene, zuclopenthixol, clozapine, olanzapine, risperidone, quetiapine, ziprasidone, amisulpride, asenapine, or paliperidone.

In one embodiment, the active ingredient(s) comprise an anti-diarrheal. In further embodiments, the anti-diarrheal is bismuth subsalicylate or loperamide.

In one embodiment, the active ingredient(s) comprise an Attention Deficit and Hyperactivity Disorder (ADHD) drug. In further embodiments, the ADHD drug is selected from any one or more of: methylphenidate, dextroamphetamine sulfate, amphetamine, or atomoxetine hydrochloride.

In one embodiment, the active ingredient(s) comprise an anti-Parkinson disease drug. In further embodiments, the anti-Parkinson disease drug is selected from any one or more of: amantadine, Apokyn, apomorphine, bromocriptine, carbidopa/levodopa, Cycloset, Duopa, entacapone/levodopa/carbidopa, Gocovri, levodopa, Mirapex, Mirapex ER, Neupro, Parlodel, pramipexole, Requip, Requip XL, ropinirole, rotigotine, Rytary, Sinemet, Sinemet CR, or Stalevo.

In one embodiment, the active ingredient(s) comprise a benzodiazepine. In further embodiments, the benzodiazepine is selected from any one or more of: alprazolam, bromazepam, chlordiazepoxide, clorazepate, diazepam, estazolam, flurazepam, halazepam, ketazolam, lorazepam, nitrazepam, oxazepam, prazepam, quazepam, temazepam, and triazolam.

In one embodiment, the active ingredient(s) comprise is a benzodiazepine antagonist. In further embodiments, the benzodiazepine antagonist is flumazenil.

In one embodiment, the active ingredient(s) comprise a barbiturate. In further embodiments, the barbiturate is selected from any one or more of: amobarbital, aprobarbotal, butabarbital, butalbital, methohexital, mephobarbital, metharbital, pentobarbital, phenobarbital, and secobarbital.

In one embodiment, the active ingredient(s) comprise a barbiturate antagonist. In further embodiments, the barbiturate is an amphetamine.

In one embodiment, the active ingredient(s) comprise a stimulant. In further embodiments, the stimulant is selected from caffeine or an amphetamine, such as amphetamine, dextroamphetamine resin complex, dextroamphetamine, methamphetamine, or methylphenidate.

In one embodiment, the active ingredient(s) comprise a stimulant antagonist. In further embodiments, the stimulant antagonist is a benzodiazepine.

In one embodiment, the active ingredient(s) comprise an antidepressant. In further embodiments, the antidepressant is selected from any one or more of: agomelatine, Allegron (see nortriptyline), amitriptyline, Anafranil (see clomipramine), Brintellix (see vortioxetine), Cipralex (see escitalopram), Cipramil (see citalopram), citalopram, clomipramine, Cymbalta (see duloxetine), Depefex XL (see venlafaxine), dosulepin, doxepin, duloxetine, Edronax (see reboxetine), Efexor XL (see venlafaxine), escitalopram, Faverin (see fluvoxamine), fluoxetine, fluvoxamine, Foraven XL (see venlafaxine), imipramine, isocarboxazid, lofepramine, Lomont (see lofepramine), Lustral (see sertraline), Manerix (see moclobemide), mianserin, mirtazapine, moclobemide, Molipaxin (see trazodone), Nardil (see phenelzine), nortriptyline, Oxactin (see fluoxetine), Parnate (see tranylcypromine), paroxetine, phenelzine, Politid XL (see venlafaxine), Prothiaden (see dosulepin), Prozac (see fluoxetine), Prozep (see fluoxetine), reboxetine, Seroxat (see paroxetine), sertraline, Sinepin (see doxepin), Sunveniz XL (see venlafaxine), Surmontil (see trimipramine), Tofranil (see imipramine), Tonpular XL (see venlafaxine), tranylcypromine, trazodone, trimipramine, Triptafen, Valdoxan (see agomelatine), Venadex XL (see venlafaxine), Venaxx XL (see venlafaxine), venlafaxine, Venlalic XL (see venlafaxine), ViePax (see venlafaxine), vortioxetine, Zispin (see mirtazapine). In preferred embodiments, the antidepressant is selected from any one or more of: citalopram (Celexa), escitalopram (Lexapro), fluoxetine (Prozac), fluvoxamine (Luvox), paroxetine (Paxil), sertraline (Zoloft), desvenlafaxine (Pristiq), duloxetine (Cymbalta), levomilnacipran (Fetzima), milnacipran (Ixel, Savella), venlafaxine (Effexor), reboxetine (Edronax), teniloxazine (Lucelan, Metatone), or viloxazine (Vivalan).

In one embodiment, the active ingredient(s) comprise a nutraceutical. In further embodiments, the nutraceutical is selected from any one or more of: 5-methyltetrahydrofolic acid, ademetionine, adenine, adenosine monophosphate, alfacalcidol, alpha-linolenic acid, ATP, beta carotene, biotin, calcidiol, calcitriol, castor oil, cholecalciferol, choline, chondroitin sulfate, coenzyme A, coenzyme Q10, creatine, curcumin, cyanocobalamin, cystine, dihomo-gamma-linolenic acid, ephedra, ergocalciferol, eucalyptol, fish oil, folic acid, Ginkgo biloba, ginkgolide-A, ginkgolide-B, ginkgolide-C, ginkgolide-J, ginkgolide-M, ginseng, ginsenoside C, ginsenoside Rb1, ginsenoside Rg1, glutamic acid, glutathione, glycine, glycine betaine, histidine, hyperforin, icosapent, icosapent ethyl, inulin, kava, krill oil, L-Alanine, L-Arginine, L-Asparagine, L-Aspartic Acid, L-Citrulline, L-Cysteine, L-Glutamine, L-Isoleucine, L-Leucine, L-Lysine, L-Phenylalanine, L-Proline, L-Threonine, L-Tryptophan, L-Tyrosine, L-Valine, lipoic acid, lutein, melatonin, menadione, methionine, N-Acetylglucosamine, NADH, niacin, octacosanol, omega-3 fatty acids, omega-6 fatty acids, ornithine, oxitriptan, oxogluric acid, pantothenic acid, phosphatidyl serine, phosphocreatine, prasterone, pyridoxal, pyridoxal phosphate, pyridoxine, pyruvic acid, riboflavin, sage oil, serine, serotonin, sesame oil, sinecatechins, spermine, St. John's Wort, succinic acid, taurine, tetrahydrofolic acid, thiamine, tretinoin, tyramine, ubidecarenone, ubiquinol, vitamin A, vitamin C, vitamin D, vitamin E, or vitamin K.

In other embodiments, one or more active ingredients may be selected from the following ingredients based on indication, classification, or site of action:

Skin health/beauty: calcium, chromium, selenium, zinc, ascobyl pulminate, magnesium, L-carnitine, N-acetyL-L-carnitine, L-glutamine, collagen hydrolysate, tumeric, dmae (dimethylaminoethanol), green tea, grape seed, alpha lipoic acid, aloe vera extract, coenzymeq10, walnut, pomegranate, botanical gelatin, polyphenols, flavonoids;

Sleep: melatonin, L-Theanine;

Cholesterol: policosanols;

CNS and brain function: Vinpocetine, Ginkgo Biloba, L-Arginine, Acetyl-L-Carnitine, Feverfew, DMAE (Dimethylaminoethanol), DMAE bitartrate, P-chlorophenoxyacetate;

Bone: coral calcium, magnesium, Vitamin K, boron; Digestive: tarragon oil, amylase, proteases, lipase, cellulose, pectin, HCL, sucrase, maltase, lactase, probiotics;

Energy: Vitamin B-complex, ginseng, Ginkgo biloba, caffeine, theobromine; Hormone: DHEA (Dehydroepiandrosterone), pregnenolone, melatonin; Weight Loss: Hoodia gordonii, Gymnema sylvestre, hydroxycitrate: green tea leaf extract, betaine, piperine, potassium, maltodextrin, Vitamin C, Vitamin E, thiamin, riboflavin, niacinamide, pyridoxine hydrochloride, biotin, chromium, molybdenum, Garcinia Cambogia, Congugated Linoleic Acid (CLA), Glucosol, Guarana, Hawthorn, ECGC (epigallocatechin-3-gallate);

Prostate: nettle root, saw palmetto, pygeum, lysopene;

Joint: MSM (dimethylsulfone), glucosamine chondroitin;

Liver Detox: N-Acetyl Cystene, Milk, Thistle, Green Tea, Alpha Lipoic Acid, Red Clover,

Multi-Vitamin: Vitamins A, C, D3, E, B1, B2, B3, B6, B12, folic acid pantothenic acid, biotin, calcium, iodine, magnesium, zinc, selenium, manganese, chromium, molybdenum, potassium, inositol;

Immune: green tea extract, colostrum, indole 3 carbonal, shiiake mushroom, grapefruit seed extract, beta 1-3 glucon;

Eye: L-taurine, N-acetyl cysteine, alpha lipoic acid, bilberry, lycopene, astazanthin, lutein; Heart: alpha lipoic acid, co-enzyme Q10, grape seed extract, hawthorne extract, L-taurine;

Male Libido: L-arginine, Muira puama, Avena sativa, Tribulis terristris, choline, Ginkgo biloba;

Female Libido: pantothenic acid, L-arginine, Muira puama, maca root, Avena sativa, dong quai, choline, Ginkgo biloba;

Mood: 5 HTP (5-hydroxytryptophan), L-theanine; Post Memopausal: black cohash, dong quai, chastertree berry, green tea, red clover, indole 3 carbinol;

Body Building: Androstenedione, L-glutamine, L-tyrosine, L-arginine, L-glycine, L-lysine, whey protein, DHEA (Dehydroepiandrosterone);

Antioxidant: Vitamin C, Vitamin E, grape seed, alpha lipoic acid, green tea; Hangover: pharmaceutical charcoal, calcium.

In one embodiment, the nutraceutical is selected from the group consisting of: folic acid, B-6, K-1, Co-Q, green tea, echinacea, myrrh or other medicinal oils, and derivatives of seaweed or kelp. In another embodiment, the nutraceuticals is selected from the group consisting of: vitamins, minerals, trace minerals, amino acids, antioxidants, alpha lipoic acid, CoQ10, DMAE, SAMe, phospholipids, choline, triglycerides, and hormones such as pregnenolone, DHEA, melatonin, naturally derived estrogen and progesterone. In another embodiment, the nutraceutical is a plants or plant component selected from the group consisting of: garlic, Ginkgo biloba, kava kava, noni, ginseng, saw palmetto, milk thistle, stinging nettle, eucalyptus, aloe vera, feverfew, nasturtium, Ma Huang, and echinacea.

In one embodiment, the active ingredient(s) comprise nicotine.

In another embodiment, the active ingredient(s) comprise a BCS Class II active ingredient. In further embodiments, the BCS Class II active ingredient is selected from any one or more of following: aceclofenac, albendazole, amiodarone, atorvastatin, azithromycin, bicalutamide, bisacodyl, cabergoline, candesartancilexetil, carbamazepine, carvedilol, cefditoren, celecoxib, chloroquine, chlorpromazine, cilostazol, ciprofloxacin, cisapride, clarithromycin, clofazimine, clopidogrel, clozapine, cyclosporine, cyproterone, danazol, dapsone, diazepam, diclofenac, diflunisal, digoxin, diloxanide, ebastine, efavirenz, epalrestat, eprosartan, erythromycin, ethylicosapentate, ezetimibe, fenofibrate, flurbiprofen, furosemide, gefitinib, gliclazide, glimepiride, glipizide, glyburide, glyburide(glibenclamide), griseofulvin, haloperidol, hydroxyzine, ibuprofen, imatinib, indinavir, indomethacin, irbesartan, isotretinoin, itraconazole, ketoconazole, ketoprofen, lamotrigine, lansoprazolei, lopinavir, loratadine, lorazepam, lovastatin, mebendazole, medroxyprogesterone, meloxicam, menatetrenone, metaxalone, metoclopramide, mosapride, mycophenolatemofetil, nabumetone, naproxen, nelfinavir, nevirapine, nicergoline, niclosamide, nifedipine, nimesulide, ofloxacin, olanzapine, orlistat, oxaprozin, phenazopyridine, phenytoin, pioglitazone, piroxicam, pranlukast, praziquantel, pyrantel, pyrimethamine, quetiapine, quinine, raloxifene, rebamipide, retinol, rifampicin, risperidone, ritonavir, rofecoxib, saquinavir, simvastatin, sirolimus, spironolactone, sulfasalazine, tacrolimus, talinolol, tamoxifen, telmisartan, teprenone, terfenadine, ticlopidine, tocopherolnicotinate, tosufloxacin, triflusal, ursodeoxycholicacid, valproicacid, valsartan, verapamil, warfarin, or zaltoprofen.

In another embodiment, at least one active ingredient is a BCS Class IV active ingredient. In further embodiments, the BCS Class IV active ingredient is selected from any one or more of following: acetaminophen (paracetamol), acetazolamide, acetylsalicylic acid, acyclovir, allopurinol, aluminium hydroxide, amoxicillin, azathioprine, cefdinir, cefixime, cefotiam, cefpodoxime, cefuroxime axetil, dapsone, dexamethasone, doxycycline, famotidine, folic acid, hydrochlorothiazide, L-carbocysteine, levodopa, linezolid, mesalamine, methylphenidate, metronidazole, modafinil, nalidixic acid, nitrofurantoin, nystatin, oxcarbazepine, oxycodone, phenobarbital, propylthiouracil, roxithromycin, sulfadiazine, sulfamethoxazole, sulpiride, sultamicillin, theophylline, or trimethoprim.

In one embodiment, at least one active ingredient is ethyl pyruvate. Ethyl pyruvate is a redox analogue of dimethyl fumarate (Tecfidera), a drug for treating multiple sclerosis treatment. Ethyl pyruvate efficiently suppressed the release of MS signature cytokines, IFN-γ and IL-17, from human PBMCs in vitro and from encephalitogenic T cells after in vivo application of EP to rats. Djordje Miljković, et al. J Immunol 194 (6) 2493-2503 (2015). Ethyl pyruvate has also been described for use in treating reperfusion injury (WO01/24793), some inflammatory disorders (WO02/074301, US2003/0232884), and renal failure (WO02/081020).

In another embodiment, the active ingredient(s) is selected from one or more of group consisting of: ace-inhibitors, anti-Alzheimer's agents, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-emetics, anti-histamines, anti-hypertensive drugs, anti-inflammatory agents, anti-lipid agents, anti-manics, anti-migraines, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, anxiolytics, anti-psychotics, appetite stimulants, biological response modifiers, blood modifiers, bone metabolism regulators, bronchodilators, cardiovascular agents, central nervous system stimulates, cholinesterase inhibitors, contraceptives, decongestants, dietary supplements, dopamine receptor agonists, endometriosis management agents, enzymes, erectile dysfunction agents, fertility agents, gastrointestinal agents, H2-antagonists, homeopathic remedies, hormones, hypercalcemia and hypocalcemia management agents, immunomodulators, immunosuppressives, migraine preparations, motion sickness treatments, muscle relaxants, non-steroidal anti-inflammatories (NSAID's), obesity management agents, osteoporosis preparations, oxytocics, parasympatholytics, parasympathomimetics, prostaglandins, psychotherapeutic agents, respiratory agents, sedatives, serotonin 5-HT3 receptor antagonists, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, anti-obesity drugs, erythropoietic drugs, anti-asthmatics, cough suppressants, mucolytics, DNA and genetic modifying drugs, and combinations thereof.

In another embodiment, the active ingredient is selected from one or more of the following: acetylcholine agonists, acromegaly agents, AIDS/HIV adjunct agents, alcohol dependence preparations, amyotrophic lateral sclerosis therapeutic agents, acetaminophen, centrally acting analgesic, narcotics, narcotic agonist-antagonist, narcotics, nonsteroidal anti-inflammatory drugs (NSAIDS), salicylates, aspirin, general anaesthetics, local anaesthetics, anticonvulsants barbiturates, benzodiazepines, Gaba analogues, hydantoins, anticonvulsants, phenyltriazines, antidiabetic agents (including biguanides, glucosidase inhibitors, insulins intermediate acting insulins, intermediate and rapid acting insulin, long acting insulins, rapid acting insulins, meglitinides, sulfonylureas, or thiazolidinediones), acetaminophen antagonists, antivenins, benzodiazepine antagonists, chelating agents, digoxin antagonists, antifibrosis therapy, antihistamines, anti-infective agents, aids adjunct anti-infectives (including non-nucleoside reverse transcriptase inhibitors nucleoside reverse transcriptase inhibitors, nucleotide analogue reverse transcriptase inhibitors or protease inhibitors), anthelmintics, antibiotics, aminoglycosides or lactam antibiotics, cephalosporins, macrolides, penicillins, quinolones, sulphonamides, tetracyclines, antifungals, antimalarial agents, antiprotozoal agents, antituberculosis agents, antivirals, leprostatics, urinary anti-infectives, antineoplastics (including alkylating agents, alkylating agents, nitrogen mustards, or nitrosoureas, antimetabolites, cytotoxic agents, hormonal agonists/antagonists, multi-kinase inhibitor, immunomodulators, or antiestrogens), estrogens, gonadotropin releasing hormone (GNRH) analogues, photosensitizing agents, skin & mucous membrane steroids, antiparkinsonian agents, catechol-o-methyltransferase, agents inhibitors, dopamine agonists, dopaminergic agents, monoamine oxidase inhibitors (MAOI), antirheumatic agents antirheumatic agents, biologicals, alphal-proteinase inhibitor, antitoxins & antivenins, immune serums, vaccines, blood modifiers anticoagulants, antiplatelet agents, colony stimulating factors, granulocyte (G-C SF), granulocyte macrophage (GM-CSF), hematinics, erythropoiesis stimulants, folic acid derivatives & iron, hemostatics, systemic hemostatics, plasma fractions, human albumin, antihemophilic factor, anti-inhibitor coagulant complex, antithrombin III, factor IX complex, immune globulin, plasma protein fraction, selective factor XA inhibitor, thrombin inhibitors, thrombolytic agents, vitamin K, bone metabolism regulators, cardioprotective agents, adrenergic blockers, peripheral & adrenergic stimulants, central & alpha/beta adrenergic blockers, angiotensin converting enzyme (ACE) inhibitors, angiotensin converting enzyme (ACE) inhibitors with calcium channel blockers, angiotensin converting enzyme (ACE) inhibitors with diuretics, angiotensin II receptor antagonists, angiotensin II receptor antagonists with diuretics, group I antiarrhythmics, antiarrhythmics, antihypertensive agents, antilipidemic agents, bile acid sequestrants, cholesterol absorption inhibitors, fibric acid derivatives, HMG-COA reductase inhibitors, antilipidemic agents, nicotinic acid agents, beta adrenergic blocking agents, beta adrenergic blocking agents with diuretics, calcium channel blockers, diuretics, loop diuretics, potassium-sparing diuretics, thiazides & related diuretics, endothelin receptor antagonist, inotropic agents, vasodilators, coronary vasodilators, natriuretic peptides, pulmonary vasodilators, vasopressors, vasoprotective agents, central nervous system depressant, amphetamines, appetite suppressants, cholinesterase inhibitors, amino acids, blood modifiers, iron, digestive aids, fiber supplements, herbal immune system support, minerals & electrolytes, calcium, magnesium, multiminerals, phosphorous, potassium, zinc, prenatal formulations, multivitamins, multivitamins with minerals, vitamin A, B vitamins, vitamin C, vitamin D analogues, vitamin E, dopamine receptor agonists, antacids (ingredients calcium carbonate alone or in combination with magnesium hydroxide, and/or aluminum hydroxide, or H2-antagonists), antidiarrheals (e.g., loperamide), antiemetics, antiflatulents, anti-inflammatory agents, antispasmodics & anticholinergics, bowel evacuants, cytoprotective agents, digestive enzymes, duodenal ulcer adherent complex, histamine (H2) receptor antagonists, laxatives, bulk producing laxatives, emollient laxatives, enemas, saline laxatives, stimulant laxatives, proton pump inhibitors, homeopathic remedies, androgens, calcitonin, estrogens, glucocorticoids, glucose elevating agents, gonadotropin inhibitors, gonadotropin releasing hormones (GNRH), gonadotropin releasing hormones (GNRH) analogues, gonadotropins, menotropins, urofollitropins, growth factor, growth hormone, growth hormone receptor antagonist, progestins, somatostatin analogues, thyroid preparations, synthetic T3 or T4, synthetic T3, synthetic T4, vasopressin & derivatives, hypercalcemia management agent, hypocalcemia management agent, hyponatremia management agent, immunomodulators, immunosupressives, medical foods, migraine preparations, ergot derivatives, serotonin (5-HT) receptor agonists, motion sickness products, multiple sclerosis management agent, muscle relaxants, acetylcholine inhibitors muscle relaxants, neuromuscular blocking agents, skeletal muscle relaxants, nasal preparations (including antibiotics, anticholinergics, anti-inflammatory agents, steroidal anti-inflammatory agents, hormones, sympathomimetics), obesity management agent, appetite suppressants, lipase inhibitors, ophthalmic agent (including acetylcholine blocking agents, antihistamine & mast cell stabilizer, quinolones, sulfonamides & anti-inflammatory agents nonsteroidal anti-inflammatory drugs (NSAIDS), steroidal anti-inflammatory agents & artificial tears/lubricants & beta adrenergic blocking agents, beta adrenergic blocking agent & carbonic anhydrase inhibitor, photodynamic therapy agents, prostaglandins, vasoconstrictors or sympathomimetics), osteoporosis agent, bisphosphonates, calcitonin, otic preparations (including antibiotic & steroids), patent ductus arteriosus agents, phosphate binders, porphyria agents, prostaglandins, antianxiety agents, benzodiazepines, antidepressants, monoamine oxidase, inhibitors (MAOI), selective serotonin reuptake inhibitors (SSRI), tricyclic antidepressants, antipanic agents, antipsychotic agents, phenothiazines, bipolar agents, obsessive-compulsive disorder management agents, antitussives, narcotic antitussives, non-narcotic antitussives, bronchodilators, anticholinergics, anticholinergics with sympathomimetics, sympathomimetics, xanthine derivatives, decongestants, expectorants, leukotriene antagonists, leukotriene formation inhibitors, lung surfactants, cold & cough products with analgesics, sedatives & hypnotics, barbiturates, benzodiazepines, acne preparations, analgesics, anesthetics, anorectal preparations, antihistamines (e.g., chlorpheniramine maleate, dextromethorphan, or pseudoephedrine HCl), anti-infectives, antibiotics, antifungals, antivirals, anti-infectives, antineoplastics, antiperspirants, antipruritics, antipsoriatic agents, burn preparations deodorants, drying agents, emollients & moisturizers, enzymes, hair growth stimulants, keratolytics, skin & mucous membrane agents including canker sore preparations, dental preparations, lozenges & sprays, mouth & throat products, oral rinses, saliva products, photosensitizing agents, scar tissue treatment, steroids, wart preparations, wound care products, smoking cessation aids, urinary tract agents including acidifiers, alkalinizers, analgesics, antibacterials, antispasmodics, benign prostatic hyperplasia (BPH) therapy, calcium oxalate stone prevention, cytoprotective agents, impotence agents, urinary tract agents, vaginal preparations including anti-infectives, estrogens, prostaglandins, or vasodilators, including cerebral vasodilators.

In another embodiment, the active ingredient is selected from one or more of: adrenergic agonists such as clonidine; anxiolytics such as alprazolam (available as Xanax®); anti-psychotics such as clozapine (available as Clozaril®) and haloperidol (available as Haldol®); non-steroidal anti-inflammatories (NSAID's) such as dicyclofenac (available as Voltaren®) and etodolac (available as Lodine®), anti-histamines such as loratadine (available as Claritin®), astemizole (available as Hismanal®), nabumetone (available as Relafen®), fexofenadine (available as Allegra®), and clemastine (available as Tavist®); anti-emetics such as granisetron hydrochloride (available as Kytril®), serotonin 5-HT3 receptor antagonists such as ondansetron (available as Zofran®) and nabilone (available as Cesamet™); bronchodilators such as salbutamol (aka albuterol, available as Ventolin®), albuterol sulfate (available as Proventil®); anti-depressants such as fluoxetine hydrochloride (available as Prozac®), sertraline hydrochloride (available as Zoloft®), and paroxetine hydrochloride (available as Paxil®); anti-migraines such as sumatriptan (available as Imigran®), ACE-inhibitors such as enalapril (available as Vasotec®), captopril (available as Capoten®) and lisinopril (available as Prinivil® and Zestril®); anti-Alzheimer's agents, such as nicergoline; calcium channel blocker (CCB) such as nifedipine (available as Procardia® and Adalat®), and verapamil hydrochloride (available as Calan®); opioid analgesics such as fentanyl (available as Sublimaze®), alfentanil, sufentanil, remifentanil, carfentanil, and lofentanil; cough suppressants such as dextromethorphan; local anesthetics such as benzocaine (available as Cepacol® and Anbesol®); peptide hormones such as insulin; oral contraceptives such as estrogen (estradiol) and a progestogen (progestin); vaccines such as killed vaccines (e.g., influenza vaccine, cholera vaccine, bubonic plague vaccine, polio vaccine, hepatitis A vaccine, and rabies vaccine); attenuated vaccines (e.g., yellow fever, measles, rubella, and mumps); toxoid vaccines (e.g., tetanus and diphtheria); subunit vaccines (e.g., subunit vaccine against Hepatitis B virus, virus-like particle (VLP) vaccine against human papillomavirus, and the hemagglutinin and neuraminidase subunits of the influenza virus); fluoridating agents such as sodium fluoride, sodium monofluorophosphate (MFP) and stannous fluoride; stimulants such as caffeine, theobromine, theophylline, yohimbine, and nicotine; energy boosters such as methylxanthines (e.g., caffeine), B vitamins (e.g., Vitamin B12), herbs, guarana, yerba mate, acai, taurine, various forms of ginseng, maltodextrin, inositol, carnitine, creatine, glucuronolactone, Ginkgo biloba, bitter orange extract, coenzyme Q10, amino acids (e.g., L-carnitine), bee pollen, royal jelly, green tea extract, spirulina, gotu kola, and glucose; opioid antidiarrheals such as loperamide (available as Imodium®); sports supplements such as fish oil, dietary protein, creatine, caffeine, glutamine, essential fatty acids (e.g., (alpha-linolenic acid and linoleic acid), prohormones (e.g., chrysin and 4-androstene-3,6,17-trione), and testosterone boosters (e.g., Fenugreek, Eurycoma longifolia, D-Aspartic acid, Boron, L-Carnitine and Tribulus terrestris); analgesics such as non-steroidal anti-inflammatory drugs (NSAIDs); COX-2 inhibitors such as rofecoxib, celecoxib and etoricoxib; opiates such as morphine, diacetylmorphine, codeine, oxycodone, hydrocodone, dihydromorphine, pentazocine, butorphanol, and pethidine; dietary supplements such as melatonin (N-acetyl-5-methoxytryptamine), vitamins, minerals, fiber, fatty acids, and amino acids; electrolytes such as sodium (NO, potassium (K+), calcium (Ca2+), magnesium (Mg2+), chloride (Cl−), hydrogen phosphate (HPO4 2−), hydrogen carbonate (HCO3−), erectile dysfunction therapies (including, sildenafil, tadalafil, vardenafil, apomorphine, yohimbine and alprostadil), ondansetron (available as Zuplenz® and Zofran®), diphenhydramine (available as Benadryl®), simethicone (available as Gas-X®), melatonin (available as MelatoninPM®), benzocaine (available as Orajel®); buprenorphine and naloxone (available as Suboxone®), buprenorphine (available as Subutex®), phenylephrine or pseudoephedrine (available as Sudafed®), acetaminophen, chlorpheniramine maleate, dextromethorphan hydrobromide, and pseudoephedrine hydrochloride (available as Theraflu®), and paracetamol and phenylephrine hydrochloride (available as Lemsip®).

In another embodiment, the composition comprises at least two active ingredients selected from: one or more cannabinoids, and/or ethyl pyruvate, and/or one or more terpenes.

In one embodiment, said composition comprises one or more cannabinoids and ethyl pyruvate. In another embodiment, said composition comprises ethyl pyruvate and one or more terpenes. In another embodiment, said composition comprises one or more cannabinoids and one or more terpenes. In another embodiment, said composition comprises one or more cannabinoids, ethyl pyruvate, and one or more terpenes. In another embodiment, said composition comprises at least two cannabinoids. In another embodiment, said composition comprises at least two terpenes. In another embodiment, said composition comprises one or more cannabinoids and at least one other active ingredient; ethyl pyruvate and at least one other active ingredient; or one or more terpenes and at least one other active ingredient.

In another embodiment, the composition comprises THC, CBD, melatonin and, optionally, one or more selected from CBN, limonene and/or beta-myrcene. In one embodiment, the composition comprises THC, CBD, melatonin, and CBN; THC, CBD, melatonin, CBN, limonene and beta-myrcene, THC, CBD, melatonin, CBN, and limonene, THC, CBD, melatonin, limonene and beta-myrcene, or THC, CBD, melatonin, CBN, and beta-myrcene.

In one embodiment, the combined active ingredients in a composition of the present invention has synergistic activity, as compared to the additive activity of equivalent compositions comprising each active ingredient alone.

In one embodiment, the composition comprises a one or more of a cannabinoid, cannabinoid extract, terpene, terpene extract, or a combination thereof. In some embodiments, the composition further comprises a surfactant. Some of the compositions of the present invention form emulsions, preferably nanoemulsions, microemulsions, or micelle dispersions in an aqueous medium. Preferably, the dispersion or emulsion is transparent at 0.1 wt % in an aqueous medium, e.g., water. In a further embodiment, the composition is a rapid dispersing formulation, forming a transparent dispersion/emulsion within a time selected from: 5, 10, 15, 20, 25, 30, 35, 40, 45, 50, 55 or 60, or 90 seconds, or within 2, 2.5, 3, 3.5, 4, 4.5, or 5 minutes after addition to an aqueous medium at a temperature selected from: 4 degrees, 20 degrees, 40 degrees, or 60 degrees C., wherein the final concentration of the composition in the aqueous medium is 0.1 wt %. In the further embodiment, the rapid dispersing formulation does not require agitation (e.g., shaking or stirring) to form a transparent dispersion or emulsion within a time selected from: 5, 10, 15, 20, 25, 30, 35, 40, 45, 50, 55 or 60, or 90 seconds, or within 2, 2.5, 3, 3.5, 4, 4.5, or 5 minutes after addition to an aqueous medium at a temperature selected from: 4 degrees, 20 degrees, 40 degrees, or 60 degrees C., wherein the final concentration of the composition in the aqueous medium is 0.1 wt %. In a further embodiment, the composition, form a transparent dispersion or emulsion within 3 min, 90 sec or 60 sec at 20 degrees C. In one embodiment, the compositions of the present invention that are rapid dispersing formulations are added to an aqueous medium dropwise using a dropper or dropper bottle or as a single bolus.

In another embodiment, the composition is a non-aqueous formulation, i.e., the composition does not contain water. In certain embodiments, the composition comprises less than; 10 wt %, 9 wt %, 8 wt %, 7 wt %, 6 wt %, 5 wt %, 4 wt %, 3 wt %, 2 wt %, 1 wt %, 0.5 wt %, 0.25 wt %, 0.1 wt %, or 0.05 wt % water.

In another embodiment, the composition is a pharmaceutical composition. In another embodiment, the composition or pharmaceutical composition is an oral dosage form, e.g., a liquid, a solid or a semi-solid oral dosage form. Another embodiment includes a unit dose (or serving) of the composition.

In one embodiment, the active ingredient is an extract from a cannabis plant (“cannabis extract”). Cannabis plants belong to the family Cannabaceae, preferably Cannabis sativa, Cannabis indica, or Cannabis hybrid. The cannabis extract may comprise one or more cannabinoids, or terpenes or other actives. In another embodiment, the cannabis extract comprises a cannabinoid, i.e., a “cannabinoid extract”. In another embodiment, the terpene is in the form of an extract from a cannabis or other plant comprising a terpene, i.e., a “terpene extract”. In a further embodiment, the cannabis, cannabinoid, or terpene extract is from a cannabis plant selected from Cannabis sativa, Cannabis indica, or Cannabis hybrid. In one embodiment, the cannabis, cannabinoid or terpene extract is an extract of Cannabis sativa. In another embodiment, the cannabis, cannabinoid or terpene extract is an extract of Cannabis indica. In another embodiment, the cannabis, cannabinoid or terpene extract is an extract of Cannabis hybrid. In another embodiment, the cannabis, cannabinoid or terpene extract is a distillate. In a further embodiment, the cannabinoid distillate is the product of short path distillation of a cannabinoid extract. In a further embodiment, the cannabinoid or terpene is synthetic.

In further embodiments, the cannabinoid extract comprises total cannabinoid(s) in an amount selected from: 50-75 wt %, 50-99 wt %, 75-99 wt %, 75-95 wt %, 80-99 wt %, 85-99 wt %, 90-99 wt %, 85-95 wt %, 90-95 wt %, or >99 wt % total cannabinoid(s). In further embodiments, the total concentration of cannabinoid(s) in a composition of the present invention is 1-200 mg/mL. In further embodiments, the total concentration of cannabinoid(s) in a composition of the present invention is selected from: 1-5 mg/mL, 1-10 mg/mL, 1-50 mg/mL, 1-100 mg/mL, 5-50 mg/mL, 10-50 mg/mL, 10-100 mg/mL, 5-10 mg/mL, 10-15 mg/mL, 15-20 mg/mL, 20-30 mg/mL, 30-40 mg/mL, 40-50 mg/mL, 50-75 mg/mL, 75-100 mg/mL, 100-150 mg/mL, or 150-200 mg/mL. In another embodiment, the total concentration of cannabinoid(s) in a composition of the present invention is <0.001 mg/mL, 0.001-0.01 mg/mL, or 0.01-1 mg/mL. In another embodiment, the total concentration of cannabinoid(s) in a composition of the present invention is selected from: 0.01-1 wt %, 0.5-1 wt %, 1-2.5 wt %, 2.5-5 wt %, 5.7.5 wt %, 7.5-10 wt %, 10-12.5 wt %, 12.5-15 wt %, 7.5-15 wt %, 10-15 wt %, 10-20 wt %, 20-30 wt %, 30-40 wt %, 40-50 wt %, or >50 wt %.

In one embodiment, the composition comprises at least one terpene. In one embodiment, the terpene is found in Cannabis sativa, Cannabis indica, or Cannabis hybrid. In a further embodiment, the terpene is extracted from a plant species, preferably a species of Cannabis (e.g., Cannabis sativa, Cannabis indica, Cannabis hybrid or other). In a further embodiment, the terpene is synthetic. In a further embodiment, the terpene is selected from any, one, two, three, four, five, six, seven, eight, nine, ten, eleven, twelve, thirteen, fourteen, fifteen, sixteen, seventeen, eighteen, nineteen, twenty, or more of the group consisting of: alpha-bisabolol, alpha-phellandrene, alpha-pinene, alpha-terpinene, alpha-terpineol, beta-caryophyllene, beta-pinene, borneol, cadinene, camphene, camphor, carvacrol, caryophyllene acetate, caryophyllene oxide, cedrane, citral, citronellol, dextro carvone, dextro fenchone, eucalyptol (1,8-cineole), eugenol, farnesene, gama-3-carene, gamma-terpinene, geraniol, geranyl acetate, guaiene, humulene, isopulegol, limonene, linalool, linalyl acetate, menthol, myrcene, nerol, nerolidol, ocimene, ocimene, p-cymene, phytol, pulegone, terpineol, terpinen-4-ol, terpinolele, terpinolene, thymol, valencene, valencene, 1-menthol, and combinations thereof.

In another embodiment, the composition further comprises at least one terpene. In a further embodiment, the at least one terpene is any one, two, three, four, five, six, or all six terpenes selected from the group consisting of beta-caryophyllene, linalool, limonene, alpha-pinene, eucalyptol, and myrcene. In a further embodiment, the at least one terpene is any one, two, three, four, or all five selected from beta-caryophyllene, linalool, limonene, alpha-pinene, or eucalyptol. In one embodiment, the composition comprises beta-caryophyllene. In another embodiment, the composition comprises myrcene. In another embodiment, the composition comprises linalool. In another embodiment, the composition comprises limonene. In another embodiment, the composition comprises alpha-pinene. In another embodiment, the composition comprises eucalyptol. In another embodiment, the composition comprises beta-caryophyllene, linalool, limonene, alpha-pinene, and eucalyptol.

The surfactants of the present invention include pharmaceutically acceptable or food grade surfactants. Surprisingly, some compositions comprising high concentrations of surfactant, including compositions containing no exogenously added fatty acid, monoglyceride, diglyceride, triglyceride, particularly, no added MCT or LCT, performed as well or better than formulations comprising an MCT or LCT.

In some embodiments, the surfactant has an HLB value greater than 9, 10, 11, 12, 13, 14, 15, 16, or greater than 16. In other embodiments, the surfactant has an HLB value between 9-17, 9-16.7, 9-16, 9-15, 9-14, 10-17, 10-16.7, 10-16, 10-15, 12-14, 12-16, 14-16, 14-17, 15-17, and between 10-14. In a preferred embodiment, the surfactant has an HLB value between 14-16, In a further preferred embodiment, the surfactant has an HLB value of about 15. In another embodiment, the composition comprises at least two surfactants, independently, with an HLB value selected from 9, 10, 11, 12, 13, 14, 15, 16, greater than 16, 9-17, 9-16.7, 9-16, 9-15, 9-14, 10-17, 10-16.7, 10-16, 10-15, 12-14, 12-16, 14-16, 14-17, 15-17, and between 10-14.

In some embodiments, the surfactant is selected from: PEG 15 hydroxystearate (Solutol HS15), polyoxyl-10-Oleyl Ether (BRIJ® 97), polyethylene glycol 25 hydrogenated castor oil, polyethylene glycol (PEG) 40 hydrogenated castor oil (Kolliphor RH40, Cremophor RH40), polyethylene-polypropylene glycol (poloxamer 124), PEG 8 caprylic/capric glycerides (Labrasol), PEG 300 oleic glycerides (Labrafil M 1944), diethylene glycol monoethyl ether (Transcutol), lauroyl macrogol 32 glycerides (GELUCIRE® 44/14), polyethylene glycol 400 (PEG 400), propylene glycol laurate (Lauroglycol FCC), D-α-Tocopherol polyethylene glycol 1000 succinate (TPGS), polyethylene-polypropylene glycol (poloxamer 188), polyethylene-polypropylene glycol (poloxamer 407), polyvinyl pyrrolidone (e.g., Mw 28-34 kDa, Mw 44-54 kDa (e.g., Kollidon 30), or 1-1.5M kDa (e.g., Kollidon 90), Iota Carrageenan, Xanthan gum, locust Bean gum, Kelcogel LT100, acacia gum, guar gum, gamma-Cyclodextrin, Tracacanth gum, hydroxypropyl methylcellulose (HPMC), carboxymethyl cellulose (CMC), microcrystalline cellulose (MCC), lecithin, polyethylene-polypropylene glycol (poloxamer 124), polyethylene glycol sorbitan monolaurate (polysorbate 20, TWEEN 20), polyethylene glycol sorbitan monopalmitate (polysorbate 40, TWEEN 40), polyethylene glycol sorbitan monostearate (polysorbate 60, TWEEN 60), polyethylene glycol sorbitan tristearate (polysorbate 65, TWEEN 65), polyethylene glycol sorbitan monooleate (polysorbate 80, TWEEN 80), polyethylene glycol sorbitan trioleate (polysorbate 85, TWEEN 85), polyethylene glycol sorbitan hexaoleate, polyethylene glycol sorbitan tetraoleate, sorbitan monolaurate (Span 20), sorbitan monopalmitate (Span 40), sorbitan monostearate (Span 60), sorbitan tristearate (Span 65), sorbitane monooleate (Span 80), sorbitan trioleate (Span 85), sucrose laurate, sucrose palmitate, sucrose stearate, gamma-cyclodextrin, beta-cyclodextrin (e.g., CAPTISOL) pectin, whey protein, caseinates, quillaia/quillaj a saponins, quillaia extract, PEG 8 stearate, PEG 40 stearate, or a combination thereof.

In other embodiments, the surfactant is selected from: polyoxyl-10-Oleyl Ether (BRIJ® 97), polyethylene glycol 25 hydrogenated castor oil, polyethylene glycol (PEG) 40 hydrogenated castor oil (Kolliphor RH40, Cremophor RH40, Croduret 40), polyethylene-polypropylene glycol (poloxamer 124), PEG 8 caprylic/capric glycerides (Labrasol), PEG 300 oleic glycerides (Labrafil M 1944), diethylene glycol monoethyl ether (Transcutol), sorbitane monooleate (Span 80), Lauroyl macrogol 32 glycerides (GELUCIRE® 44/14), polyethylene glycol 400 (PEG 400), propylene glycol laurate (Lauroglycol FCC), polysorbate 20 (TWEEN® 20), polysorbate 40 (TWEEN® 40), polysorbate 60 (TWEEN® 60), polysorbate 80 (TWEEN® 80), D-α-Tocopherol polyethylene glycol 1000 succinate (TPGS), polyethylene-polypropylene glycol (poloxamer 188), polyethylene-polypropylene glycol (poloxamer 407), polyvinyl pyrrolidone (Kollidon 30), polyvinyl pyrrolidone (Kollidon 90), Iota Carrageenan, Xanthan gum, locust Bean gum, Kelcogel LT100, acacia gum, guar gum, gamma-Cyclodextrin, Tracacanth gum, hydroxypropyl methylcellulose (HPMC), carboxymethyl cellulose (CMC), microcrystalline cellulose (MCC), lecithin, or a combination thereof.

In other embodiments, the surfactant is selected from: Lauroyl macrogol 32 glycerides (GELUCIRE® 44/14), polyethylene glycol 400 (PEG 400), propylene glycol laurate (Lauroglycol FCC), polysorbate 20 (TWEEN® 20), polysorbate 40 (TWEEN® 40), polysorbate 60 (TWEEN® 60), polysorbate 80 (TWEEN® 80), D-α-Tocopherol polyethylene glycol 1000 succinate (TPGS), polyethylene-polypropylene glycol (poloxamer 188), polyethylene-polypropylene glycol (poloxamer 407), polyvinyl pyrrolidone (Kollidon 30), polyvinyl pyrrolidone (Kollidon 90), Iota Carrageenan, Xanthan gum, locust Bean gum, Kelcogel LT100, acacia gum, guar gum, gamma-Cyclodextrin, Tracacanth gum, hydroxypropyl methylcellulose (HPMC), carboxymethyl cellulose (CMC), microcrystalline cellulose (MCC), lecithin, or a combination thereof.

In further embodiments, the surfactant is selected from: Lauroyl macrogol 32 glycerides (GELUCIRE® 44/14), polyethylene glycol 400 (PEG 400), propylene glycol laurate (Lauroglycol FCC), polysorbate 20 (TWEEN® 20), polysorbate 40 (TWEEN® 40), polysorbate 60 (TWEEN® 60), polysorbate 80 (TWEEN® 80), D-α-Tocopherol polyethylene glycol 1000 succinate (TPGS), polyethylene-polypropylene glycol (poloxamer 188), polyethylene-polypropylene glycol (poloxamer 407), polyvinyl pyrrolidone (Kollidon 30), polyvinyl pyrrolidone (Kollidon 90), or a combination thereof.

In a further embodiment, the surfactant is TPGS and/or lauroyl macrogol 32 glycerides (e.g., GELUCIRE® 44/14). In another further embodiment, the surfactant is polysorbate 80. In a further embodiment, the surfactant is polyethylene glycol (PEG) 40 hydrogenated castor oil (Kolliphor RH40) and/or polysorbate 80. In a further embodiment, the surfactant is TPGS and polysorbate 80. In a further embodiment, the surfactant is polyethylene glycol (PEG) 40 hydrogenated castor oil (Kolliphor RH40). Surprisingly, polyethylene glycol (PEG) 40 hydrogenated castor oil unexpectedly better at solubilizing cannabinoids as compared with polysorbate 80. For example, compositions generally required 3-5 times less polyethylene glycol (PEG) 40 hydrogenated castor oil compared with polysorbate 80 to form a transparent emulsion or micellar dispersion in water.

In some embodiments, the composition comprises at least one co-solvent. In one embodiment, the co-solvent is selected from at least one of: ethanol, ethyl lactate, ethyl olelate, glycerol, or propylene glycol. In a further embodiment, said co-solvent is selected from at least one of: ethanol, ethyl lactate, or propylene glycol. In one embodiment, the co-solvent is ethanol. In some embodiments, the composition comprises an active ingredient, e.g., cannabinoid or cannabinoid extract, a surfactant, and a co-solvent(s) in an amount selected from: 0-2.5 wt %, 2.5-5 wt %, 5-10 wt %, 10-15 wt %, 15-20 wt %, 20-25 wt %, 25-30 wt %, 30-35 wt %, 35-40 wt %, 40-45 wt %, 45-50 wt %, 50-55 wt %, 55-60 wt %, 60-65 wt %, 65-70 wt %, 70-75 wt %, 75-80 wt %, 80-85 wt %, 85-90 wt %, 90-95 wt %, or 95-97 wt % co-solvent(s). In a further embodiment, said co-solvent(s) is present in an amount selected from 15-40 wt %, 15-25 wt %, 20-30 wt %, or 25-35 wt %. In a further embodiment, the composition comprises 20-30 wt %, 20-25 wt %, or 25-30 wt % co-solvent(s).

In one embodiment, a composition comprises a cannabinoid or cannabinoid extract, polysorbate 80, and a co-solvent, e.g., ethanol, wherein said composition comprises at least about 25, 30, or 35 wt % co-solvent and a wt % ratio of cannabinoid or cannabinoid extract:polysorbate 80 that is at least 1:7-11, 1:8-10, or at least about 1:9. In another embodiment, a composition comprises a cannabinoid or cannabinoid extract, polyethylene glycol (PEG) 40 hydrogenated castor oil, and a co-solvent, e.g., ethanol, wherein said composition comprises at least about 25 wt % co-solvent and a wt % ratio of cannabinoid or cannabinoid extract:polyethylene glycol (PEG) 40 hydrogenated castor oil that is at least 1:1-2, 1:2-5, 1:1.5-4, 1:2-4, or at least 1:2-3. In a further embodiment, the co-solvent is present in an amount that is about 20-99%, 25-99%, 20-50%, 25-75%, 25-50%, 25-40%, 25-35%. In a further embodiment, the co-solvent is ethanol.

In some embodiments, the composition comprises an active ingredient, e.g., cannabinoid or cannabinoid extract and a surfactant, wherein the surfactant is in an amount selected from: at least 5 wt %, at least 10 wt %, at least 15 wt %, at least 20 wt %, at least 25 wt %, at least 30 wt %, at least 35 wt %, at least 40 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, at least 97 wt %, 0-2.5 wt %, 2.5-5 wt %, 5-10 wt %, 10-15 wt %, 15-20 wt %, 20-25 wt %, 25-30 wt %, 30-35 wt %, 35-40 wt %, 40-45 wt %, 45-50 wt %, 45-55 wt %, 45-65 wt %, 46-65 wt %, 47-65 wt %, 48-65 wt %, 49-65 wt %, 50-65 wt %, 51-65 wt %, 52-65 wt %, 53-65 wt %, 54-65 wt %, 55-65 wt %, 50-55 wt %, 55-60 wt %, 60-65 wt %, 65-70 wt %, 70-75 wt %, 75-80 wt %, 80-85 wt %, 85-90 wt %, 90-95 wt %, or 95-97 wt % surfactant. In some embodiments, the surfactant has an HLB value greater than 9, greater than 10, between 9-17, between 9-16.7, between 9-16, between 9-15, between 10-17, between 10-16.7, between 10-16, between 10-15, between 10-14, between 9-13.4, between 14-16, between 14-17, between 15-17, or between 10-13.4. In a preferred embodiment, the surfactant has an HLB value of between 12-13, 13-14, 14-15, or 15-16. In a further preferred embodiment, the surfactant has an HLB value of about 12, 13, 14, or 15. In one embodiment, the active ingredient is selected from a cannabinoid, cannabinoid extract, terpene, or terpene extract. In a preferred embodiment, the composition comprises an active ingredient, e.g., cannabinoid or cannabinoid extract, and at least 40%, at least 45%, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, or at least 97 wt %, 35-40 wt %, 40-45 wt %, 45-50 wt %, 45-55 wt %, 45-65 wt %, 46-65 wt %, 47-65 wt %, 48-65 wt %, 49-65 wt %, 50-65 wt %, 51-65 wt %, 52-65 wt %, 53-65 wt %, 54-65 wt %, 55-65 wt %, 50-55 wt %, 55-60 wt %, 60-65 wt %, 65-70 wt %, or 70-75 wt % surfactant, wherein the surfactant has an HLB value greater than 11, greater than 11.2, greater than 12, greater than 12.4, greater than 12.6, greater than 13, greater than 13.3, 11-12, 12-13, 12.4-16.7, 12.4-16, 13-14, or 14-15. In another preferred embodiment, the surfactant has an HLB value of about 12-15, 12-13, 12.5-13.5, 13.5-14.5, or 14.5-15.5. In another preferred embodiment, the active ingredient is selected from a cannabinoid, cannabinoid extract, terpene, or terpene extract.

Self-emulsifying drug delivery systems (SEDDS) provides a means to enhance the dissolution of some actives in an aqueous environment. Examples of patents demonstrating the potential use of SEDDS or lipid delivery systems for lipophilic drugs include U.S. Pat. Nos. 5,484,801; 5,798,333; 5,965,160; 6,008,228; 6,730,330; 9,265,724; U.S. Patent Application No. 20050209345; 20060160888; US20140357708; 20160184258; and PCT Publications WO96/39142 and WO2016147186. U.S. Pat. No. 9,265,724 and U.S. Patent Application 20160184258 exemplify a few SEDDS formulations comprising 49 THC. The present invention includes improved formulations comprising at least one active ingredient and at least one fatty acid, at least one monoglyceride, at least one diglyceride, or at least one triglyceride, including improved SEDDS formulations.

In one embodiment, the composition comprises:

at least one active ingredient;

at least one fatty acid, at least one monoglyceride, at least one diglyceride, or at least one triglyceride, or a combination thereof; and, optionally,

at least one surfactant.

In one embodiment, the active ingredient is selected from a cannabinoid, cannabinoid extract, terpene, or terpene extract.

In another embodiment, the composition comprises:

a cannabinoid or cannabinoid extract and at least one surfactant.

In another embodiment, the composition comprises:

at least one active ingredient;

at least one surfactant; and, optionally,

at least one fatty acid, at least one monoglyceride, at least one diglyceride, or at least one triglyceride, or a combination thereof.

In one embodiment, the active ingredient is selected from a cannabinoid, cannabinoid extract, terpene, or terpene extract.

In one embodiment, the composition comprises at least one fatty acid. In another embodiment, the composition comprises at least one monoglyceride. In another embodiment, the composition comprises at least one diglyceride. In another embodiment, the composition comprises at least one triglyceride. In other embodiments, the composition comprises at least one fatty acid and at least one monoglyceride; at least one fatty acid and at least one diglyceride; at least one fatty acid and at least one triglyceride; at least one monoglyceride and at least one diglyceride; at least one monoglyceride and at least one triglyceride; at least one diglyceride and at least one triglyceride; at least one fatty acid, at least one monoglyceride, at least one diglyceride, and at least one triglyceride; or at least one monoglyceride, at least one diglyceride, and at least one triglyceride.

In one embodiment, the fatty acid, monoglyceride, diglyceride, triglyceride, or a combination thereof is an oil. In a further embodiment, the oil is selected from anise oil, apricot kernel oil PEG-6 esters, apricot kernel oil, beeswax, borage oil, canola oil, castor oil, polyoxyl 35 castor oil, polyoxyl 40 hydrogenated castor oil, polyoxyl 40 castor oil, polyoxyl 60 hydrogenated castor oil, hydrogenated castor oil, polyoxyl 60 castor oil, cinnamon oil, clove oil, coconut oil fractioned, coconut oil, coconut oil-lecithin, coriander oil, corn oil PEG-6 esters, corn oil PEG-8 esters, corn oil, cottonseed oil hydrogenated, cottonseed oil, cottonseed oil, hydrogenated soybean oil, hydrogenated vegetable oils, kernel oil PEG-6 esters, kernel oil, lemon oil, mineral oil (light), mineral oil, neutral oil, nutmeg oil, olive oil PEG-6 esters, olive oil, orange oil, palm kernel oil PEG-6 esters, palm kernel oil, palm kernel oil/palm kernel oil hydrogenated, palm fruit oil, peanut oil PEG-6 esters, peanut oil, peppermint oil, poppy seed oil, safflower oil, soybean oil hydrogenated, soybean oil refined, soybean oil, sunflower oil, triisostearin PEG-6 esters, vegetable oil hydrogenated, vegetable oil PEG esters, vegetable oil, vegetable oils glyceride hydrogenated, or a mixture thereof.

In one embodiment, the fatty acid, monoglyceride, diglyceride, triglyceride, or a combination thereof is a fat. In another embodiment, the fatty acid, monoglyceride, diglyceride, triglyceride, or a combination thereof is exogenously added fatty acid, monoglyceride, diglyceride, triglyceride, or a combination thereof. The term “exogenously added”, as used herein, means other than any fatty acids, monoglycerides, diglycerides, triglycerides, or combinations thereof, that were originally present in a cannabis plant, or other plant extract, and remains in the extract, e.g., a cannabinoid extract, after the extraction/distillation process. For clarity, pressed cannabis/hemp seed oil added to a composition of the present invention is exogenously added. In one embodiment, the only exogenously added fatty acid, monoglyceride, diglyceride, triglyceride, or a combination thereof, is a flavoring oil, e.g., flavor compounds diluted with and MCT or other oil. In a further embodiment, the flavoring oil is an essential oil. In a further embodiment, the essential oil is produced by distillation (e.g., steam distillation), solvent extraction (example, a hydrocarbon such as hexane or supercritical carbon dioxide), or by expression.

In one embodiment, the cannabinoid extract is essentially free of fatty acids, monoglycerides, diglycerides, or triglycerides. In a further embodiment, the cannabinoid extract is essentially free of fatty acids. In another embodiment, the cannabinoid extract is essentially free of monoglycerides. In another embodiment, the cannabinoid extract is essentially free of diglycerides. In another embodiment, the cannabinoid extract is essentially free of triglycerides. In another embodiment, the composition is essentially free of exogenously added fatty acids. In another embodiment, the composition is essentially free of exogenously added monoglycerides. In another embodiment, the composition is essentially free of exogenously added diglycerides. In another embodiment, the composition is essentially free of exogenously added triglycerides. In another embodiment, the composition is essentially free of exogenously added fats or oils.

In some embodiments, the composition comprises an active ingredient, and at least 5 wt %, at least 10 wt %, at least 15 wt %, at least 20 wt %, at least 25 wt %, at least 30 wt %, at least 35 wt %, at least 40 wt %, at least 50 wt %, at least 55 wt %, at least 60 wt %, at least 65 wt %, at least 70 wt %, at least 75 wt %, at least 80 wt %, at least 85 wt %, at least 90 wt %, at least 91 wt %, at least 92 wt %, at least 93 wt %, at least 94 wt %, or at least 95 wt % of exogenously added fat, oil, fatty acid, monoglyceride, diglyceride, triglyceride, MCT, LCT, or a combination thereof. In one embodiment, the active ingredient is selected from a cannabinoid, cannabinoid extract, terpene, or terpene extract.

In some embodiments, the composition comprises an active ingredient, and not more than: 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 25 wt %, 30 wt %, 35 wt %, 40 wt %, 50 wt %, 55 wt %, 60 wt %, 65 wt %, 70 wt %, 75 wt %, 80 wt %, 85 wt %, 90 wt %, or 95 wt % of exogenously added fat, oil, fatty acid, monoglyceride, diglyceride, triglyceride, MCT, LCT, or a combination thereof, or a combination thereof. In one embodiment, the active ingredient is selected from a cannabinoid, cannabinoid extract, terpene, or terpene extract.

In some embodiments, the composition comprises an active ingredient, and 0-2.5 wt %, 0-5 wt %, 0-10 wt %, 0-11 wt %, 0-12 wt %, 0-13 wt %, 0-14 wt %, 0-15 wt %, 0-16 wt %, 2.5-5 wt %, 5-10 wt %, 10-15 wt %, 15-20 wt %, 20-25 wt %, 25-30 wt %, 30-35 wt %, 35-40 wt %, 40-45 wt %, 45-50 wt %, 50-55 wt %, 55-60 wt %, 60-65 wt %, 65-70 wt %, 70-75 wt %, 75-80 wt %, 80-85 wt %, 85-90 wt %, 87-92 wt %, 90-95 wt %, or 91-96 wt % of exogenously added fat, oil; exogenously added fatty acid, monoglyceride, diglyceride, triglyceride, or a combination thereof; or exogenously added MCT, LCT, or a combination thereof. In one embodiment, the active ingredient is selected from a cannabinoid, cannabinoid extract, terpene, or terpene extract.

In some embodiments, the composition comprises an active ingredient, and at least 5 wt %, at least 10 wt %, at least 15 wt %, at least 20 wt %, at least 25 wt %, at least 30 wt %, at least 35 wt %, at least 40 wt %, at least 50 wt %, at least 55 wt %, at least 60 wt %, at least 65 wt %, at least 70 wt %, at least 75 wt %, at least 80 wt %, at least 85 wt %, at least 90 wt %, at least 91 wt %, at least 92 wt %, at least 93 wt %, at least 94 wt %, or at least 95 wt % of fat, oil, or combination thereof; fatty acid, monoglyceride, diglyceride, triglyceride, or a combination thereof; or MCT, LCT, or combination thereof. In one embodiment, the active ingredient is selected from a cannabinoid, cannabinoid extract, terpene, or terpene extract.

In some embodiments, the composition comprises an active ingredient, and not more than: 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 25 wt %, 30 wt %, 35 wt %, 40 wt %, 50 wt %, 55 wt %, 60 wt %, 65 wt %, 70 wt %, 75 wt %, 80 wt %, 85 wt %, 90 wt %, or 95 wt % of fat, oil, or a combination thereof; fatty acid, monoglyceride, diglyceride, triglyceride, or a combination thereof; MCT, LCT, or a combination thereof. In one embodiment, the active ingredient is selected from a cannabinoid, cannabinoid extract, terpene, or terpene extract.

In some embodiments, the composition comprises an active ingredient, and 0-2.5 wt %, 2.5-5 wt %, 5-10 wt %, 10-15 wt %, 15-20 wt %, 20-25 wt %, 25-30 wt %, 30-35 wt %, 35-40 wt %, 40-45 wt %, 45-50 wt %, 50-55 wt %, 55-60 wt %, 60-65 wt %, 65-70 wt %, 70-75 wt %, 75-80 wt %, 80-85 wt %, 85-90 wt %, 87-92 wt %, 90-95 wt %, or 91-96 wt % of fat, oil, or a combination thereof; fatty acid, monoglyceride, diglyceride, triglyceride, or a combination thereof; fat, oil, or a combination thereof. In one embodiment, the active ingredient is selected from a cannabinoid, cannabinoid extract, terpene, or terpene extract.

In another embodiment, the monoglyceride, diglyceride, or triglyceride is a medium chain monoglyceride, diglyceride, or triglyceride and/or a long chain monoglyceride, diglyceride triglyceride. In a further embodiment, the triglyceride is a medium chain triglyceride (MCT). In another further embodiment, the triglyceride is a long chain triglyceride (LCT).

In one embodiment, the composition comprises: a cannabinoid, and at least one surfactant from, polyethylene glycol (PEG) 40 hydrogenated castor oil (Kolliphor RH40), D-α-Tocopherol polyethylene glycol 1000 succinate (TPGS), polysorbate 80, or lauroyl macrogol 32 glycerides, or a combination thereof. In a further embodiment, the composition comprises a cannabinoid, TPGS, lauroyl macrogol 32 glycerides, and a MCT and/or LCT. In a further embodiment, the composition comprises a cannabinoid, TPGS, lauroyl macrogol 32 glycerides, and a MCT. In a further embodiment, the composition comprises a cannabinoid, TPGS, lauroyl macrogol 32 glycerides, and a LCT. In one embodiment, the lauroyl macrogol 32 glycerides is GELUCIRE 44/14. In a further embodiment, the composition comprises a cannabinoid, polysorbate 80, and TPGS. In a further embodiment, the composition comprises a cannabinoid, polysorbate 80, and polyethylene glycol (PEG) 40 hydrogenated castor oil (Kolliphor RH40). In a further embodiment, the composition comprises a cannabinoid and polyethylene glycol (PEG) 40 hydrogenated castor oil (Kolliphor RH40).

In some embodiments, the composition of the invention comprises: an amount of active ingredient(s) selected from 0.5-2.5 wt %, 2.5-5 wt %, 2.5-7.5 wt %, 5-7.5 wt %, 5-10 wt %, 7.5-10 wt %, 7.5-12.5 wt %, or 10-12.5 wt %; an amount of surfactant(s) selected from 35-40 wt %, 40-45 wt %, 45-55 wt %, 45-50 wt %, 46-56%, 47-57%, 48-58%, 49-59%, 45-55 wt %, 45-65 wt %, 46-65 wt %, 47-65 wt %, 48-65 wt %, 49-65 wt %, 50-65 wt %, 51-65 wt %, 52-65 wt %, 53-65 wt %, 54-65 wt %, 55-65 wt %, 50-55 wt %, 55-60 wt %, 60-65 wt %, 65-70 wt %, 70-75 wt %, or 75-80 wt %; and, an amount of co-solvent selected from 5-10 wt %, 10-15 wt %, 15-20 wt %, 20-25 wt %, 25-30 wt %, 30-35 wt %, or 35-40 wt %, 35-40 wt %, 40-45 wt %, 45-50 wt %, 50-55 wt %, 55-60 wt %, 60-65 wt %, 65-70 wt %, 70-75 wt %, 75-80 wt %, 80-85 wt %, 85-90 wt %, 90-95 wt %, or 95-99 wt %.

In some further embodiments, the composition further comprises: an amount of oil, preferably MCT, LCT, MCT and LCT, selected from: 0-5 wt %, 0-10 wt %, 0-11 wt %, 0-12 wt %, 0-13 wt %, 0-14 wt %, 0-15 wt %, 0-16 wt %, 0.5-1 wt %, 1-2 wt %, 1-2.5 wt %, 1-5 wt %, 1-10 wt %, 1-20 wt %, 2-3 wt %, 3-4 wt %, 4-5 wt %, 5-7.5 wt %, 5-10 wt %, 5-11 wt %, 5-12 wt %, 5-13 wt %, 5-14 wt %, 5-15 wt %, 5-16 wt %, 10-12.5 wt %, 10-15 wt %, 10-20 wt %, 15-20 wt %, or 20-25 wt %, 25-30 wt %, or 25-50% wt %.

In some further embodiments, the composition further comprises: an amount of flavoring or flavor agents selected from 0-1 wt %, 0-2 wt %, 0-3 wt %, 0-3.5 wt %, 0-3.75 wt %, 0-4 wt %, 0.5-1 wt %, 1-2 wt %, 1-2.5 wt %, 1-5 wt %, 2-3 wt %, 3-4 wt %, 4-5 wt %, 5-7.5 wt %, 5-10 wt %, 10-12.5 wt %, 10-15 wt %, 10-20 wt %, 15-20 wt %, or 20-25 wt %, 25-30 wt %, or 25-50% wt %. In some further embodiments, the composition further comprises: an amount of sweetener selected from 0-5 wt %, 0-7.5 wt %, 1-2 wt %, 2-3 wt %, 2.5-5%, 3-4 wt %, 4-5 wt %, 5-6 wt %, 5-7.5 wt %, 7.5-10 wt %, or 5-10 wt %. As used herein, the term “flavoring” may represent a single species of flavor agent (e.g., limonene) or a mixture of flavor agent species (e.g., limonene, linalool, citral, citronellol, geranyl acetate and perillaldehyde) combined to produce a certain flavor. The flavoring may further comprise a vehicle, e.g., MCT, for solubilizing the flavor agent(s). A “flavor agent” is a single molecule, e.g., limonene, used alone or in combination with other flavor agent(s) to produce a certain flavor, e.g., citrus or orange, of a flavoring. A flavoring is normally supplied as a concentrate for dilution and for the purpose of imparting a flavor or taste-masking a substance.

In some further embodiments, the surfactant is polysorbate 80 and/or polyethylene glycol (PEG) 40 hydrogenated castor oil (Kolliphor RH40). In some further embodiments, the co-solvent is selected from ethanol, ethyl lactate, and/or propylene glycol. In some embodiments the co-solvent is a mixture of co-solvents, e.g., ethanol and propylene glycol. In some further embodiments, the surfactant is polysorbate 80 and/or polyethylene glycol (PEG) 40 hydrogenated castor oil (Kolliphor RH40) and the co-solvent is selected from ethanol, ethyl lactate, and/or propylene glycol. In some further embodiments, at least one active ingredient is a cannabinoid, the surfactant is polysorbate 80 and/or polyethylene glycol (PEG) 40 hydrogenated castor oil (Kolliphor RH40) and the co-solvent is selected from ethanol, ethyl lactate, and/or propylene glycol.

In some further embodiments, the composition comprises: 3-5 wt % or 5-10 wt % cannabinoid or cannabinoid extract; 45-65 wt %, 50-65 wt %, 45-55 wt %, 52-62 wt %, 55-65 wt %, or >65 wt % polysorbate 80 and/or polyethylene glycol (PEG) 40 hydrogenated castor oil (Kolliphor RH40); 15-30 wt %, 20-25 wt %, 20-30 wt %, 20-35 wt %, 35-45 wt %, or >45 wt % co-solvent selected from ethanol, ethyl lactate, and/or propylene glycol; 2.5-7.5%, 2.5-5 wt %, or 2-4 wt % sweetener(s) (e.g., sucralose); 1-2 wt %, 2-3 wt %, 2.5-5%, 3-4 wt %, 4-5 wt %, 5-6 wt %, 5-7.5 wt %, 7.5-10 wt %, or 5-10 wt %, 5-10 wt %, 10-15% wt %, 7-17 wt %, 15-20 wt %, or 20-25 wt % flavoring or flavoring agent, and 0 wt %, <5 wt %, <10 wt %, <15 wt %, <20 wt %, <25 wt %, 1-5 wt %, 1-10 wt %, 1-20 wt %, 5-10 wt %, 10-15 wt %, 10-20 wt %, 15-20 wt %, or 20-25 wt %. Unexpectedly, the addition ethanol, ethyl lactate, and/or propylene glycol as a co-solvent to a composition comprising polysorbate 80 and/or polyethylene glycol (PEG) 40 hydrogenated castor oil (Kolliphor RH40) significantly reduced the dissolution time from hours to minutes or seconds. These rapid dispersion formulations also require significantly less polyethylene glycol (PEG) 40 hydrogenated castor oil than polysorbate 80.

In some further embodiments, an aqueous emulsion comprising 0.1-0.2 vol. % of a composition of the invention is transparent, with an estimated average particle size ≤50 nm, ≤75 nm, ≤100 nm, ≤125 nm, ≤150 nm, ≤200 nm, ≤250 nm.

In some embodiments, the composition comprises polysorbate 80 and TPGS. One surprising advantage of these compositions is a significant reduction or elimination of leakage when unsealed hard gelatin capsules are filled with the composition. The formulations, when added to an aqueous medium, have fast dissolution rates and form transparent mirco- or nanoemulsions. In further embodiments, the formulations comprise 30-50 wt % polysorbate 80, 50-70 wt % TPGS, and 1-20 wt % active ingredient. In further embodiments, the formulations comprise 40-50 wt % polysorbate 80, 40-50 wt % TPGS, and 1-20 wt % active ingredient. In further embodiments, the formulations comprise 30-50 wt % polysorbate 80, 50-70 wt % TPGS, and 5-15 wt % cannabinoid or cannabinoid extract. In further embodiments, the formulations comprise 40-50 wt % polysorbate 80, 40-50 wt % TPGS, and 5-20 wt % cannabinoid or cannabinoid extract. In further embodiments, the formulations comprise 40-50 wt % polysorbate 80, 40-50 wt % TPGS, and 5-10% cannabinoid or cannabinoid extract. In further embodiments, the formulations comprise 40-50 wt % polysorbate 80, 40-50 wt % TPGS, and 5-10 wt % cannabinoid or cannabinoid extract. In a further embodiment, the composition consists of 45 wt % polysorbate 80, 45% TPGS, and 10 wt % THC-distillate.

In another embodiment, the composition comprises an active ingredient, and polysorbate 80. In one embodiment, at least one active ingredient is selected from a cannabinoid, cannabinoid extract, terpene, or terpene extract. In a further embodiment, the composition consists of an active ingredient, and polysorbate 80. In one embodiment, at least one active ingredient is selected from a cannabinoid, cannabinoid extract, terpene, or terpene extract. In a further embodiment, the composition comprises an active ingredient, polysorbate 80 and a MCT and/or LCT. In one embodiment, at least one active ingredient is selected from a cannabinoid, cannabinoid extract, terpene, or terpene extract. In a further embodiment, the composition comprises an active ingredient, polysorbate 80 and an MCT. In a further embodiment, the composition comprises an active ingredient, polysorbate 80 and an LCT. In one embodiment, at least one active ingredient is selected from a cannabinoid, cannabinoid extract, terpene, or terpene extract.

In another embodiment, the composition comprises CBD, ethyl pyruvate, optionally THC, optionally at least one terpene, and polysorbate 80. and/or polyethylene glycol (PEG) 40 hydrogenated castor oil. In one embodiment, the composition further comprises THC and/or one or more terpene. In another embodiment, the terpene in selected from any one, two, three, four, five, or all six of the terpenes selected from the group consisting of: myrcene, beta-caryophyllene, linalool, limonene, alpha-pinene, and eucalyptol. In a further embodiment, the composition comprises one, two, three, four, or all five terpenes selected from the group consisting of beta-caryophyllene, linalool, limonene, alpha-pinene, and eucalyptol. In a further embodiment, the composition consists of CBD, ethyl pyruvate, THC, one or more terpene, and polysorbate 80 and/or polyethylene glycol (PEG) 40 hydrogenated castor oil. In one embodiment, the composition comprises a cannabinoid extract or terpene extract. In a further embodiment, the composition comprises CBD, ethyl pyruvate, THC, one or more terpene, polysorbate 80 and/or polyethylene glycol (PEG) 40 hydrogenated castor oil, and a MCT and/or LCT. In one embodiment, the composition comprises a cannabinoid extract or terpene extract. In a further embodiment, the composition comprises CBD, ethyl pyruvate, THC, one or more terpene, polysorbate 80 and/or polyethylene glycol (PEG) 40 hydrogenated castor oil, and an MCT. In one embodiment, the composition comprises a cannabinoid extract and/or terpene extract.

In another embodiment, the composition comprises THC, CBD, melatonin and,

optionally, one or more selected from CBN, D-limonene and/or beta-myrcene. In one embodiment, the composition comprises: (a) THC, CBD, melatonin, and CBN; (b) THC, CBD, melatonin, CBN, limonene and beta-myrcene; (c) THC, CBD, melatonin, CBN, and limonene; (d) THC, CBD, melatonin, limonene and beta-myrcene; or (e) THC, CBD, melatonin, CBN, and beta-myrcene. In a further embodiment, a unit dose of one of the above compositions comprises 0-10, 0-5, 1-9, 2-8, 3-7, 4-6 or about 5 mg THC; 20-60, 30-50, 35-45, 37.5-42.5 or about 40 mg CBD; 0-10, 0-5, 1-9, 2-8, 3-7, 4-6 or about 5 mg CBN (if present); 0-13, 4-12, 5-11, 6-10, 7-9, 7.5-8.5, or about 8 mg D-limonene (if present); 0-10, 0.25-6-8, 0.5-6, 0.5-4, 1.0-3.5, 1.0-3, 1.5-2.5, or about 2 mg beta-myrcene (if present); and 0-10, 0.25-7, 0.25-5, 1-5, 1-2.5, 0.25-2.5, 0.25-2, 0.5-2, 1-2, or about 0.25-1 mg melatonin. In a further embodiment, the composition comprises about 5 mg THC, about 40 mg CBD, about 5 mg CBN, about 0.25-1 mg melatonin, about 8 mg D-limonene, and about 2 mg beta-myrcene.

In another embodiment, the composition comprises at least one active ingredient;

a MCT and/or LCT;

a first surfactant; and

a second surfactant;

wherein the wt % of active ingredient, MCT and/or LCT, first surfactant, and second surfactant (where the first and second surfactant are different) is selected from one of the compositions in Table 1 below. Each of the composition in Table 1 is an individual embodiment of the present invention.

TABLE 1

| | | | | | | Flavoring | |
|---|---|---|---|---|---|---|---|
| | Active | MCT and/or | First | Second | Co-solvent | or flavor | Sweetener |
| No. | ingredient wt % | LCT wt % | surfactant wt % | surfactant wt % | wt % | agent(s) wt % | wt % |
| 1 | 1-15 | 0-85 | 5-85 | 5-85 | 0-35 or ≥25 | 0-7.5 | 0-7.5 |
| 2 | 1-15 | 65-75 | 0-15 | 0-15 | 0-35 or ≥25 | 0-7.5 | 0-7.5 |
| 3 | 1-15 | 75-90 | 0-15 | 0-15 | 0-35 or ≥25 | 0-7.5 | 0-7.5 |
| 4 | 1-15 | 50-65 | 5-15 | 5-15 | 0-35 or ≥25 | 0-7.5 | 0-7.5 |
| 5 | 1-15 | 65-85 | 5-15 | 5-15 | 0-35 or ≥25 | 0-7.5 | 0-7.5 |
| 6 | 1-15 | 65-85 | 6-12 | 6-12 | 0-35 or ≥25 | 0-7.5 | 0-7.5 |
| 7 | 8-12 | 68-76 | 7-11 | 7-11 | 0-35 or ≥25 | 0-7.5 | 0-7.5 |
| 8 | 9-11 | 70-74 | 8-10 | 8-10 | 0-35 or ≥25 | 0-7.5 | 0-7.5 |
| 9 | 10 | 72 | 9 | 9 | 0-35 or ≥25 | 0-7.5 | 0-7.5 |
| 10 | 1-15 | 25-40 | 5-25 | 5-25 | 0-35 or ≥25 | 0-7.5 | 0-7.5 |
| 11 | 1-15 | 40-85 | 5-25 | 5-25 | 0-35 or ≥25 | 0-7.5 | 0-7.5 |
| 12 | 1-15 | 25-40 | 15-25 | 15-25 | 0-35 or ≥25 | 0-7.5 | 0-7.5 |
| 13 | 1-15 | 40-65 | 15-25 | 15-25 | 0-35 or ≥25 | 0-7.5 | 0-7.5 |
| 14 | 1-15 | 20-35 | 20-25 | 20-25 | 0-35 or ≥25 | 0-7.5 | 0-7.5 |
| 15 | 1-15 | 35-60 | 20-25 | 20-25 | 0-35 or ≥25 | 0-7.5 | 0-7.5 |
| 16 | 8-15 | 40-45 | 20-25 | 20-25 | 0-35 or ≥25 | 0-7.5 | 0-7.5 |
| 17 | 1-15 | 35-75 | 10-35 | 10-35 | 0-35 or ≥25 | 0-7.5 | 0-7.5 |
| 18 | 1-15 | 5-25 | 25-35 | 25-35 | 0-35 or ≥25 | 0-7.5 | 0-7.5 |
| 19 | 1-15 | 25-45 | 25-35 | 25-35 | 0-35 or ≥25 | 0-7.5 | 0-7.5 |
| 20 | 1-15 | 0-5 | 35-45 | 35-45 | 0-35 or ≥25 | 0-7.5 | 0-7.5 |
| 21 | 1-15 | 5-25 | 35-45 | 35-45 | 0-35 or ≥25 | 0-7.5 | 0-7.5 |
| 22 | 1-15 | <25 | 35-45 | 35-45 | 0-35 or ≥25 | 0-7.5 | 0-7.5 |
| 23 | 1-15 | <20 | 35-45 | 35-45 | 0-35 or ≥25 | 0-7.5 | 0-7.5 |
| 24 | 1-15 | <15 | 35-45 | 35-45 | 0-35 or ≥25 | 0-7.5 | 0-7.5 |
| 25 | 1-15 | <10 | 35-45 | 35-45 | 0-35 or ≥25 | 0-7.5 | 0-7.5 |
| 26 | 1-15 | <5 | 35-45 | 35-45 | 0-35 or ≥25 | 0-7.5 | 0-7.5 |
| 27 | 1-15 | <2.5 | 35-45 | 35-45 | 0-35 or ≥25 | 0-7.5 | 0-7.5 |
| 28 | 1-15 | 0-5 | 45-50 | 45-50 | 0-35 or ≥25 | 0-7.5 | 0-7.5 |
| 29 | 1-15 | 0-10 | 50-60 | 25-45 | 0-35 or ≥25 | 0-7.5 | 0-7.5 |
| 30 | 1-15 | 10-20 | 50-60 | 25-45 | 0-35 or ≥25 | 0-7.5 | 0-7.5 |
| 31 | 1-15 | 0-10 | 60-70 | 15-35 | 0-35 or ≥25 | 0-7.5 | 0-7.5 |
| 32 | 1-15 | 10-20 | 60-70 | 15-35 | 0-35 or ≥25 | 0-7.5 | 0-7.5 |
| 33 | 1-15 | 35-65 | 15-35 | 15-35 | 0-35 or ≥25 | 0-7.5 | 0-7.5 |
| 34 | 1-15 | 35-65 | 15-30 | 15-30 | 0-35 or ≥25 | 0-7.5 | 0-7.5 |
| 35 | 1-15 | 0-10 | 70-80 | 5-25 | 0-35 or ≥25 | 0-7.5 | 0-7.5 |
| 36 | 1-15 | 10-20 | 70-80 | 5-25 | 0-35 or ≥25 | 0-7.5 | 0-7.5 |
| 37 | 1-15 | 0-5 | 80-90 | 0-15 | 0-35 or ≥25 | 0-7.5 | 0-7.5 |
| 38 | 1-15 | 5-15 | 80-90 | 0-15 | 0-35 or ≥25 | 0-7.5 | 0-7.5 |
| 39 | 1-15 | 0-2.5 | 90-95 | 0-5 | 0-35 or ≥25 | 0-7.5 | 0-7.5 |
| 40 | 1-15 | 2.5-5 | 90-95 | 0-5 | 0-35 or ≥25 | 0-7.5 | 0-7.5 |
| 41 | 1-15 | 0-10 | 25-45 | 50-60 | 0-35 or ≥25 | 0-7.5 | 0-7.5 |
| 42 | 1-15 | 10-20 | 25-45 | 50-60 | 0-35 or ≥25 | 0-7.5 | 0-7.5 |
| 43 | 1-15 | 0-10 | 15-35 | 60-70 | 0-35 or ≥25 | 0-7.5 | 0-7.5 |
| 44 | 1-15 | 10-20 | 15-35 | 60-70 | 0-35 or ≥25 | 0-7.5 | 0-7.5 |
| 45 | 1-15 | 0-10 | 5-25 | 70-80 | 0-35 or ≥25 | 0-7.5 | 0-7.5 |
| 46 | 1-15 | 5-20 | 5-25 | 70-80 | 0-35 or ≥25 | 0-7.5 | 0-7.5 |
| 47 | 1-15 | 0-5 | 0-15 | 80-90 | 0-35 or ≥25 | 0-7.5 | 0-7.5 |
| 48 | 1-15 | 5-15 | 0-15 | 80-90 | 0-35 or ≥25 | 0-7.5 | 0-7.5 |
| 49 | 1-15 | 0-2.5 | 0-5 | 90-95 | 0-35 or ≥25 | 0-7.5 | 0-7.5 |
| 50 | 1-15 | 2.5-5 | 0-5 | 90-95 | 0-35 or ≥25 | 0-7.5 | 0-7.5 |
| 51 | 1-15 | 0-85 | 5-85 | 5-85 | 0-35 or ≥25 | 0-7.5 | 0-7.5 |
| 52 | 1-15 | 0-75 | 10-85 | 10-85 | 0-35 or ≥25 | 0-7.5 | 0-7.5 |
| 53 | 1-15 | 0-65 | 15-85 | 15-85 | 0-35 or ≥25 | 0-7.5 | 0-7.5 |
| 54 | 1-15 | 0-55 | 20-85 | 20-85 | 0-35 or ≥25 | 0-7.5 | 0-7.5 |
| 55 | 1-15 | 0-45 | 25-85 | 25-85 | 0-35 or ≥25 | 0-7.5 | 0-7.5 |
| 56 | 1-15 | 0-35 | 30-85 | 30-85 | 0-35 or ≥25 | 0-7.5 | 0-7.5 |
| 57 | 1-15 | 0-25 | 35-85 | 35-85 | 0-35 or ≥25 | 0-7.5 | 0-7.5 |
| 58 | 1-15 | 0-15 | 40-85 | 40-85 | 0-35 or ≥25 | 0-7.5 | 0-7.5 |
| 59 | 1-15 | 0-10 | 42.5-85 | 42.5-85 | 0-35 or ≥25 | 0-7.5 | 0-7.5 |
| 60 | 1-15 | 0-5 | 45-85 | 45-85 | 0-35 or ≥25 | 0-7.5 | 0-7.5 |
| 61 | 1-15 | 0-5 | 50-85 | 45-85 | 0-35 or ≥25 | 0-7.5 | 0-7.5 |
| 62 | 1-15 | 0-10 | 55-85 | 40-85 | 0-35 or ≥25 | 0-7.5 | 0-7.5 |
| 63 | 1-15 | 0-10 | 60-85 | 35-85 | 0-35 or ≥25 | 0-7.5 | 0-7.5 |
| 64 | 1-15 | 0-10 | 65-85 | 30-85 | 0-35 or ≥25 | 0-7.5 | 0-7.5 |
| 65 | 1-15 | 0-10 | 70-85 | 25-85 | 0-35 or ≥25 | 0-7.5 | 0-7.5 |
| 66 | 1-15 | 0-10 | 75-85 | 15-85 | 0-35 or ≥25 | 0-7.5 | 0-7.5 |
| 67 | 1-15 | 0-10 | 45-85 | 50-85 | 0-35 or ≥25 | 0-7.5 | 0-7.5 |
| 68 | 1-15 | 0-10 | 40-85 | 55-85 | 0-35 or ≥25 | 0-7.5 | 0-7.5 |
| 69 | 1-15 | 0-10 | 35-85 | 60-85 | 0-35 or ≥25 | 0-7.5 | 0-7.5 |
| 70 | 1-15 | 0-10 | 30-85 | 65-85 | 0-35 or ≥25 | 0-7.5 | 0-7.5 |
| 71 | 1-15 | 0-10 | 25-85 | 70-85 | 0-35 or ≥25 | 0-7.5 | 0-7.5 |
| 72 | 1-15 | 0-10 | 15-85 | 75-85 | 0-35 or ≥25 | 0-7.5 | 0-7.5 |
| 73 | 1-15 | 0-10 | 10-85 | 10-85 | 0-35 or ≥25 | 0-7.5 | 0-7.5 |
| 74 | 1-15 | 10-20 | 10-85 | 10-85 | 0-35 or ≥25 | 0-7.5 | 0-7.5 |
| 75 | 1-15 | 20-30 | 10-85 | 10-85 | 0-35 or ≥25 | 0-7.5 | 0-7.5 |
| 76 | 1-15 | 30-40 | 10-85 | 10-85 | 0-35 or ≥25 | 0-7.5 | 0-7.5 |
| 77 | 1-15 | 40-50 | 10-85 | 10-85 | 0-35 or ≥25 | 0-7.5 | 0-7.5 |
| 78 | 1-15 | 50-60 | 10-85 | 10-85 | 0-35 or ≥25 | 0-7.5 | 0-7.5 |
| 79 | 1-15 | 60-75 | 10-85 | 10-85 | 0-35 or ≥25 | 0-7.5 | 0-7.5 |
| 80 | 1-15 | 0-65 | 10-65 | 10-65 | 0-35 or ≥25 | 0-7.5 | 0-7.5 |
| 81 | 1-15 | 0-15 | 10-65 | 10-65 | 0-35 or ≥25 | 0-7.5 | 0-7.5 |
| 82 | 1-15 | 15-30 | 10-65 | 10-65 | 0-35 or ≥25 | 0-7.5 | 0-7.5 |
| 83 | 1-15 | 30-45 | 10-65 | 10-65 | 0-35 or ≥25 | 0-7.5 | 0-7.5 |
| 84 | 1-15 | 45-60 | 10-65 | 10-65 | 0-35 or ≥25 | 0-7.5 | 0-7.5 |
| 85 | 1-15 | 0-55 | 10-55 | 10-55 | 0-35 or ≥25 | 0-7.5 | 0-7.5 |
| 86 | 1-15 | 0-15 | 10-55 | 10-55 | 0-35 or ≥25 | 0-7.5 | 0-7.5 |
| 87 | 1-15 | 15-30 | 10-55 | 10-55 | 0-35 or ≥25 | 0-7.5 | 0-7.5 |
| 88 | 1-15 | 30-45 | 10-55 | 10-55 | 0-35 or ≥25 | 0-7.5 | 0-7.5 |
| 89 | 1-15 | 45-55 | 10-55 | 10-55 | 0-35 or ≥25 | 0-7.5 | 0-7.5 |
| 90 | 1-15 | 0-55 | 10-35 | 10-35 | 0-35 or ≥25 | 0-7.5 | 0-7.5 |
| 91 | 1-15 | 0-15 | 10-35 | 10-35 | 0-35 or ≥25 | 0-7.5 | 0-7.5 |
| 92 | 1-15 | 15-30 | 10-35 | 10-35 | 0-35 or ≥25 | 0-7.5 | 0-7.5 |
| 93 | 1-15 | 30-45 | 10-35 | 10-35 | 0-35 or ≥25 | 0-7.5 | 0-7.5 |
| 94 | 1-15 | 30-60 | 10-35 | 10-35 | 0-35 or ≥25 | 0-7.5 | 0-7.5 |
| 95 | 1-15 | 0-25 | 10-50 | 10-50 | 0-35 or ≥25 | 0-7.5 | 0-7.5 |
| 96 | 1-15 | 0-15 | 10-50 | 10-50 | 0-35 or ≥25 | 0-7.5 | 0-7.5 |
| 97 | 1-15 | 15-25 | 10-50 | 10-50 | 0-35 or ≥25 | 0-7.5 | 0-7.5 |
| 98 | 1-15 | 30-60 | 10-35 | 10-35 | 0-35 or ≥25 | 0-7.5 | 0-7.5 |
| 99 | 1-15 | 35-55 | 15-30 | 15-30 | 0-35 or ≥25 | 0-7.5 | 0-7.5 |
| 100 | 1-15 | 0-25 | 15-50 | 15-50 | 0-35 or ≥25 | 0-7.5 | 0-7.5 |
| 101 | 1-15 | 0-10 | 15-50 | 15-50 | 0-35 or ≥25 | 0-7.5 | 0-7.5 |
| 102 | 1-15 | 15-25 | 15-50 | 15-50 | 0-35 or ≥25 | 0-7.5 | 0-7.5 |
| 103 | 1-15 | 0-10 | 15-50 | 15-50 | 0-35 or ≥25 | 0-7.5 | 0-7.5 |
| 104 | 15-25 | 0-10 | 15-50 | 15-50 | 0-35 or ≥25 | 0-7.5 | 0-7.5 |
| 105 | 25-35 | 0-10 | 15-50 | 15-50 | 0-35 or ≥25 | 0-7.5 | 0-7.5 |
| 106 | 35-50 | 0-10 | 15-50 | 15-50 | 0-35 or ≥25 | 0-7.5 | 0-7.5 |
| 107 | 1-15 | 0-16 | 45-70 | 0-25 | 0-35 or ≥25 | 0-7.5 | 0-7.5 |
| 108 | 1-15 | 0-15 | 46-70 | 0-25 | 0-35 or ≥25 | 0-7.5 | 0-7.5 |
| 109 | 1-15 | 0-14 | 47-70 | 0-25 | 0-35 or ≥25 | 0-7.5 | 0-7.5 |
| 110 | 1-15 | 0-13 | 48-70 | 0-25 | 0-35 or ≥25 | 0-7.5 | 0-7.5 |
| 111 | 1-15 | 0-12 | 49-70 | 0-25 | 0-35 or ≥25 | 0-7.5 | 0-7.5 |
| 112 | 1-15 | 0-12 | 51-70 | 0-25 | 0-35 or ≥25 | 0-7.5 | 0-7.5 |
| 113 | 1-15 | 0-11 | 52-70 | 0-25 | 0-35 or ≥25 | 0-7.5 | 0-7.5 |
| 114 | 1-15 | 0-10 | 52.5-70 | 0-25 | 0-35 or ≥25 | 0-7.5 | 0-7.5 |
| 115 | 1-15 | 0-8 | 55-70 | 0-25 | 0-35 or ≥25 | 0-7.5 | 0-7.5 |

In further embodiments, at least one active ingredient of any one composition selected from 1-115 of Table 1 is a cannabinoid, cannabinoid extract, terpene, or terpene extract. In further embodiments, at least one active ingredient is a cannabinoid. In further embodiments, at least one active ingredient is a cannabinoid extract. In further embodiments, at least one active ingredient is a terpene. In further embodiments, at least one active ingredient is a terpene extract.

In further embodiments, a composition selected from one of the compositions 1-115 of Table 1 is a non-aqueous composition.

In further embodiments, a composition selected from one of the compositions 1-115 of Table 1 is a solid or semi-solid composition.

In further embodiments, a composition selected from one of the compositions from 1-115 of Table 1 comprises: 0.01-0.1 wt %, 0.1-1 wt %, 1 wt %, 0.5-1 wt %, 1-2.5 wt %, 1-3 wt %, 2.5-5 wt %, 3-8 wt %, 5.7.5 wt %, 5-10 wt %, 7.5-10 wt %, 7.5-15 wt %, 8-15 wt %, 8-12 wt %, 9-11 wt %, more than 8 wt %, more than 10 wt %, 10-12.5 wt %, 12.5-15 wt %, 10-15 wt %, 10-20 wt %, 20-30 wt %, 30-40 wt %, 40-50 wt %, or >50 wt % of an active ingredient(s), preferably comprising a cannabinoid or cannabinoid extract. In further embodiments, a composition selected from one of the compositions from 1-6, 10-15, 17-103, and 107-115 of Table 1 comprises 1-5 wt % of an active ingredient(s), preferably comprising a cannabinoid or cannabinoid extract.

In further embodiments, the cannabinoid extract comprises total cannabinoid(s) in an amount selected from: 50-75 wt %, 50-99 wt %, 75-99 wt %, 75-95 wt %, 80-99 wt %, 85-99 wt %, 90-99 wt %, 85-95 wt %, 90-95 wt %, or >99 wt % total cannabinoid(s).

In further embodiments, the total concentration of the active ingredient, e.g., cannabinoid(s), in a composition selected from one of the compositions from 1-115 of Table 1 is 1-200 mg/mL. In further embodiments, the total concentration of the active ingredient(s), e.g., cannabinoid(s), in a composition selected from 1-115 of Table 1 is selected from: 1-5 mg/mL, 1-10 mg/mL, 1-50 mg/mL, 1-100 mg/mL, 5-50 mg/mL, 10-50 mg/mL, 10-100 mg/mL, 5-10 mg/mL, 10-15 mg/mL, 15-20 mg/mL, 20-30 mg/mL, 30-40 mg/mL, 40-50 mg/mL, 50-75 mg/mL, 75-100 mg/mL, 100-150 mg/mL, or 150-200 mg/mL. In another embodiment, the total concentration of the active ingredient(s), e.g., cannabinoid(s), in a composition selected from one of the compositions from 1-115 of Table 1 is <0.001 mg/mL, 0.001-0.01 mg/mL, or 0.01-1 mg/mL.

In further embodiments, a composition selected from 1-115 of Table 1 comprises the active ingredient(s), e.g., cannabinoid(s), in an amount selected from: 0.25-1 mg, 0.5-2.5 mg, 2.5-5 mg, 5-7.5 mg, 7.5-10 mg, 10-12.5 mg, 12.5-15 mg, 15-20 mg, 20-30 mg, 30-40 mg, 40-50 mg, 50-60 mg, 60-70 mg, or 70-75 mg. In further embodiments, the cannabinoid is THC. In other embodiments, the cannabinoids are THC and CBD. In another embodiment, a composition selected from 1-115 of Table 1 comprises <0.001 mg, 0.001-0.25 mg, or 0.25-1 mg of cannabinoid(s).

In further embodiments, a composition selected from compositions 1-115 of Table 1 comprises MCT. In further embodiments, the composition comprises MCT, but not LCT. In further embodiments, the MCT is an oil. In further embodiments, where permissible based on the ranges for a particular composition, a composition of Table 1 comprises no more than 25 wt % MCT, 20 wt % MCT, 15 wt % MCT, 10 wt % MCT, 5 wt % MCT, 3 wt % MCT, or 1 wt % MCT. In further embodiments, a composition selected from compositions 1-115 comprises LCT. In further embodiments, the composition comprises LCT but not MCT. In further embodiments, the LCT is an oil. In further embodiments, where permissible based on the ranges for a particular composition, a composition of Table 1 comprises no more than 25 wt % MCT, 20 wt % MCT, 15 wt % MCT, 10 wt % MCT, 5 wt % LCT, 3 wt % LCT, or 1 wt % LCT. In further embodiments, the composition comprises both MCT and LCT. In further embodiments, both the MCT and the LCT is an oil. In a further embodiment, a composition of Table 1, where permissible, comprises a total amount of combined MCT and LCT selected from: 0-5 wt %, 0-10 wt %, 0-11 wt %, 0-12 wt %, 0-13 wt %, 0-14 wt %, 0-15 wt %, 0-16 wt %, 0.5-1 wt %, 1-2 wt %, 1-2.5 wt %, 1-5 wt %, 1-10 wt %, 1-20 wt %, 2-3 wt %, 3-4 wt %, 4-5 wt %, 5-7.5 wt %, 5-10 wt %, 5-11 wt %, 5-12 wt %, 5-13 wt %, 5-14 wt %, 5-15 wt %, 5-16 wt %, 10-12.5 wt %, 10-15 wt %, 10-20 wt %, 15-20 wt %, or 20-25 wt %, 25-30 wt %, or 25-50% wt % combined MCT/LCT.

In further embodiments, the first surfactant of a composition selected from 1-115 of Table 1 is D-α-Tocopherol polyethylene glycol 1000 succinate (TPGS). In further embodiments, the second surfactant of a composition selected from one of the compositions 1-115 of Table 1 is lauroyl macrogol 32 glycerides. In further embodiments, for a composition selected from 1-115 of Table 1, the first surfactant is D-α-Tocopherol polyethylene glycol 1000 succinate (TPGS) and the second surfactant is lauroyl macrogol 32 glycerides. In further embodiments, the lauroyl macrogol 32 glycerides is GELUCIRE 44/14. In further embodiments, the first surfactant of a composition selected from 1-115 of Table 1 is polysorbate 80 and the second surfactant is polyethylene glycol (PEG) 40 hydrogenated castor oil (Kolliphor RH40). In one embodiment, a composition of Table 1, where permissible, comprises a total amount of surfactant selected from: 0-2.5 wt %, 2.5-5 wt %, 5-10 wt %, 10-15 wt %, 15-20 wt %, 20-25 wt %, 25-30 wt %, 30-35 wt %, 35-40 wt %, 40-45 wt %, 45-50 wt %, 45-55 wt %, 45-65 wt %, 46-65 wt %, 47-65 wt %, 48-65 wt %, 49-65 wt %, 50-65 wt %, 51-65 wt %, 52-65 wt %, 53-65 wt %, 54-65 wt %, 55-65 wt %, 50-55 wt %, 55-60 wt %, 60-65 wt %, 65-70 wt %, 70-75 wt %, 75-80 wt %, 80-85 wt %, 85-90 wt %, 90-95 wt %, or 95-97 wt % surfactant.

In another embodiment, the invention provides a composition comprising:

an active ingredient; and

polysorbate 80 (polyoxyethylene (20) sorbitan monooleate, E433) and/or polyethylene glycol (PEG) 40 hydrogenated castor oil (Kolliphor RH40). In one embodiment, the active ingredient is selected from a cannabinoid, cannabinoid extract, terpene, or terpene extract. In a further embodiment, the active ingredient is selected from a cannabinoid or cannabinoid extract. In a further embodiment, the composition further comprises a medium-chain triglyceride (MCT) or long-chain triglyceride (LCT). In a further embodiment, the MCT or LCT is an oil.

In further embodiments, the composition comprises:

at least one active ingredient;

at least one surfactant; and, optionally,

a MCT and/or a LCT;

wherein the wt % of the active ingredient, the surfactant, and the MCT and/or LCT is selected from one of the compositions in Table 2 below. Each of the compositions in Table 2 is an individual embodiment of the present invention.

TABLE 2

| | | | | | | Flavoring | |
|---|---|---|---|---|---|---|---|
| | Active | MCT | LCT | Surfactant | Co-solvent(s) | or flavor | Sweetener |
| No. | ingredient wt % | wt % | wt % | wt % | wt % | agent(s) wt % | wt % |
| 116 | 1-15 | 45-55 | 0-10 | 10-20 | 0-35 or ≥25 | 0-7.5 | 0-7.5 |
| 117 | 1-15 | 55-65 | 0-10 | 10-20 | 0-35 or ≥25 | 0-7.5 | 0-7.5 |
| 118 | 1-15 | 65-85 | 0-10 | 10-20 | 0-35 or ≥25 | 0-7.5 | 0-7.5 |
| 119 | 1-15 | 35-45 | 0-10 | 20-30 | 0-35 or ≥25 | 0-7.5 | 0-7.5 |
| 120 | 1-15 | 45-55 | 0-10 | 20-30 | 0-35 or ≥25 | 0-7.5 | 0-7.5 |
| 121 | 1-15 | 55-75 | 0-10 | 20-30 | 0-35 or ≥25 | 0-7.5 | 0-7.5 |
| 122 | 1-15 | 25-35 | 0-10 | 30-40 | 0-35 or ≥25 | 0-7.5 | 0-7.5 |
| 123 | 1-15 | 35-45 | 0-10 | 30-40 | 0-35 or ≥25 | 0-7.5 | 0-7.5 |
| 124 | 1-15 | 45-65 | 0-10 | 30-40 | 0-35 or ≥25 | 0-7.5 | 0-7.5 |
| 125 | 1-15 | 20-35 | 0-10 | 35-45 | 0-35 or ≥25 | 0-7.5 | 0-7.5 |
| 126 | 1-15 | 35-60 | 0-10 | 35-45 | 0-35 or ≥25 | 0-7.5 | 0-7.5 |
| 127 | 1-15 | 15-25 | 0-10 | 40-50 | 0-35 or ≥25 | 0-7.5 | 0-7.5 |
| 128 | 1-15 | 25-35 | 0-10 | 40-50 | 0-35 or ≥25 | 0-7.5 | 0-7.5 |
| 129 | 1-15 | 30-40 | 0-10 | 40-50 | 0-35 or ≥25 | 0-7.5 | 0-7.5 |
| 130 | 1-15 | 40-50 | 0-10 | 40-50 | 0-35 or ≥25 | 0-7.5 | 0-7.5 |
| 131 | 1-15 | 35-55 | 0-10 | 40-50 | 0-35 or ≥25 | 0-7.5 | 0-7.5 |
| 132 | 1-15 | 5-20 | 0-10 | 50-60 | 0-35 or ≥25 | 0-7.5 | 0-7.5 |
| 133 | 1-15 | 15-30 | 0-10 | 50-60 | 0-35 or ≥25 | 0-7.5 | 0-7.5 |
| 134 | 1-15 | 20-30 | 0-10 | 50-60 | 0-35 or ≥25 | 0-7.5 | 0-7.5 |
| 135 | 1-15 | 30-45 | 0-10 | 50-60 | 0-35 or ≥25 | 0-7.5 | 0-7.5 |
| 136 | 1-15 | 0-10 | 0-10 | 60-70 | 0-35 or ≥25 | 0-7.5 | 0-7.5 |
| 137 | 1-15 | 5-15 | 0-10 | 60-70 | 0-35 or ≥25 | 0-7.5 | 0-7.5 |
| 138 | 1-15 | 10-20 | 0-10 | 60-70 | 0-35 or ≥25 | 0-7.5 | 0-7.5 |
| 139 | 1-15 | 15-35 | 0-10 | 60-70 | 0-35 or ≥25 | 0-7.5 | 0-7.5 |
| 140 | 1-15 | 20-35 | 0-10 | 60-70 | 0-35 or ≥25 | 0-7.5 | 0-7.5 |
| 141 | 1-15 | 0-10 | 0-10 | 65-75 | 0-35 or ≥25 | 0-7.5 | 0-7.5 |
| 142 | 1-15 | 10-20 | 0-10 | 65-75 | 0-35 or ≥25 | 0-7.5 | 0-7.5 |
| 143 | 1-15 | 0-10 | 0-5 | 70-80 | 0-35 or ≥25 | 0-7.5 | 0-7.5 |
| 144 | 1-15 | 0-10 | 0-10 | 70-80 | 0-35 or ≥25 | 0-7.5 | 0-7.5 |
| 145 | 1-15 | 5-15 | 0-10 | 70-80 | 0-35 or ≥25 | 0-7.5 | 0-7.5 |
| 146 | 1-15 | 15-25 | 0-10 | 70-80 | 0-35 or ≥25 | 0-7.5 | 0-7.5 |
| 147 | 1-15 | 0-10 | 0-5 | 80-90 | 0-35 or ≥25 | 0-7.5 | 0-7.5 |
| 148 | 1-15 | 0-10 | 0-10 | 80-90 | 0-35 or ≥25 | 0-7.5 | 0-7.5 |
| 149 | 1-15 | 5-10 | 0-10 | 80-90 | 0-35 or ≥25 | 0-7.5 | 0-7.5 |
| 150 | 1-15 | 10-15 | 0-10 | 80-90 | 0-35 or ≥25 | 0-7.5 | 0-7.5 |
| 151 | 1-15 | 0-10 | 0-5 | 85-95 | 0-35 or ≥25 | 0-7.5 | 0-7.5 |
| 152 | 1-15 | 5-10 | 0-10 | 85-95 | 0-35 or ≥25 | 0-7.5 | 0-7.5 |
| 153 | 1-15 | 0-10 | 0-10 | 25-95 | 0-35 or ≥25 | 0-7.5 | 0-7.5 |
| 154 | 1-15 | 10-45 | 0-10 | 25-95 | 0-35 or ≥25 | 0-7.5 | 0-7.5 |
| 155 | 1-15 | 45-90 | 0-10 | 25-95 | 0-35 or ≥25 | 0-7.5 | 0-7.5 |
| 156 | 1-15 | 0-10 | 0-10 | 25-75 | 0-35 or ≥25 | 0-7.5 | 0-7.5 |
| 157 | 1-15 | 10-35 | 0-10 | 25-75 | 0-35 or ≥25 | 0-7.5 | 0-7.5 |
| 158 | 1-15 | 35-70 | 0-10 | 25-75 | 0-35 or ≥25 | 0-7.5 | 0-7.5 |
| 159 | 1-15 | 0-10 | 0-10 | 25-55 | 0-35 or ≥25 | 0-7.5 | 0-7.5 |
| 160 | 1-15 | 10-25 | 0-10 | 25-55 | 0-35 or ≥25 | 0-7.5 | 0-7.5 |
| 161 | 1-15 | 25-35 | 0-10 | 25-55 | 0-35 or ≥25 | 0-7.5 | 0-7.5 |
| 162 | 1-15 | 35-55 | 0-10 | 25-55 | 0-35 or ≥25 | 0-7.5 | 0-7.5 |
| 163 | 1-15 | 0-10 | 0-10 | 50-75 | 0-35 or ≥25 | 0-7.5 | 0-7.5 |
| 164 | 1-15 | 10-20 | 0-10 | 50-75 | 0-35 or ≥25 | 0-7.5 | 0-7.5 |
| 165 | 1-15 | 20-35 | 0-10 | 50-75 | 0-35 or ≥25 | 0-7.5 | 0-7.5 |
| 166 | 1-15 | 35-75 | 0-10 | 50-75 | 0-35 or ≥25 | 0-7.5 | 0-7.5 |
| 167 | 1-15 | 0-5 | 0-5 | 75-95 | 0-35 or ≥25 | 0-7.5 | 0-7.5 |
| 168 | 1-15 | 5-10 | 0-5 | 75-95 | 0-35 or ≥25 | 0-7.5 | 0-7.5 |
| 169 | 1-15 | 0-10 | 0-10 | 75-95 | 0-35 or ≥25 | 0-7.5 | 0-7.5 |
| 170 | 1-15 | 10-20 | 0-10 | 75-95 | 0-35 or ≥25 | 0-7.5 | 0-7.5 |
| 171 | 1-15 | 0-10 | 45-55 | 10-20 | 0-35 or ≥25 | 0-7.5 | 0-7.5 |
| 172 | 1-15 | 0-10 | 55-65 | 10-20 | 0-35 or ≥25 | 0-7.5 | 0-7.5 |
| 173 | 1-15 | 0-10 | 65-85 | 10-20 | 0-35 or ≥25 | 0-7.5 | 0-7.5 |
| 174 | 1-15 | 0-10 | 35-45 | 20-30 | 0-35 or ≥25 | 0-7.5 | 0-7.5 |
| 175 | 1-15 | 0-10 | 45-55 | 20-30 | 0-35 or ≥25 | 0-7.5 | 0-7.5 |
| 176 | 1-15 | 0-10 | 55-75 | 20-30 | 0-35 or ≥25 | 0-7.5 | 0-7.5 |
| 177 | 1-15 | 0-10 | 25-35 | 30-40 | 0-35 or ≥25 | 0-7.5 | 0-7.5 |
| 178 | 1-15 | 0-10 | 35-45 | 30-40 | 0-35 or ≥25 | 0-7.5 | 0-7.5 |
| 179 | 1-15 | 0-10 | 45-65 | 30-40 | 0-35 or ≥25 | 0-7.5 | 0-7.5 |
| 180 | 1-15 | 20-35 | 0-10 | 35-45 | 0-35 or ≥25 | 0-7.5 | 0-7.5 |
| 181 | 1-15 | 35-60 | 0-10 | 35-45 | 0-35 or ≥25 | 0-7.5 | 0-7.5 |
| 182 | 1-15 | 0-10 | 15-25 | 40-50 | 0-35 or ≥25 | 0-7.5 | 0-7.5 |
| 183 | 1-15 | 0-10 | 25-35 | 40-50 | 0-35 or ≥25 | 0-7.5 | 0-7.5 |
| 184 | 1-15 | 30-40 | 0-10 | 40-50 | 0-35 or ≥25 | 0-7.5 | 0-7.5 |
| 185 | 1-15 | 40-50 | 0-10 | 40-50 | 0-35 or ≥25 | 0-7.5 | 0-7.5 |
| 186 | 1-15 | 0-10 | 35-55 | 40-50 | 0-35 or ≥25 | 0-7.5 | 0-7.5 |
| 187 | 1-15 | 0-10 | 5-20 | 50-60 | 0-35 or ≥25 | 0-7.5 | 0-7.5 |
| 188 | 1-15 | 15-30 | 0-10 | 50-60 | 0-35 or ≥25 | 0-7.5 | 0-7.5 |
| 189 | 1-15 | 0-10 | 20-30 | 50-60 | 0-35 or ≥25 | 0-7.5 | 0-7.5 |
| 190 | 1-15 | 0-10 | 30-45 | 50-60 | 0-35 or ≥25 | 0-7.5 | 0-7.5 |
| 191 | 1-15 | 0-10 | 0-10 | 60-70 | 0-35 or ≥25 | 0-7.5 | 0-7.5 |
| 192 | 1-15 | 5-15 | 0-10 | 60-70 | 0-35 or ≥25 | 0-7.5 | 0-7.5 |
| 193 | 1-15 | 0-10 | 10-20 | 60-70 | 0-35 or ≥25 | 0-7.5 | 0-7.5 |
| 194 | 1-15 | 15-35 | 0-10 | 60-70 | 0-35 or ≥25 | 0-7.5 | 0-7.5 |
| 195 | 1-15 | 0-10 | 20-35 | 60-70 | 0-35 or ≥25 | 0-7.5 | 0-7.5 |
| 196 | 1-15 | 0-10 | 0-10 | 65-75 | 0-35 or ≥25 | 0-7.5 | 0-7.5 |
| 197 | 1-15 | 10-20 | 0-10 | 65-75 | 0-35 or ≥25 | 0-7.5 | 0-7.5 |
| 198 | 1-15 | 0-10 | 0-5 | 70-80 | 0-35 or ≥25 | 0-7.5 | 0-7.5 |
| 199 | 1-15 | 0-10 | 5-15 | 70-80 | 0-35 or ≥25 | 0-7.5 | 0-7.5 |
| 200 | 1-15 | 0-10 | 15-25 | 70-80 | 0-35 or ≥25 | 0-7.5 | 0-7.5 |
| 201 | 1-15 | 0-10 | 0-5 | 80-90 | 0-35 or ≥25 | 0-7.5 | 0-7.5 |
| 203 | 1-15 | 0-10 | 5-10 | 80-90 | 0-35 or ≥25 | 0-7.5 | 0-7.5 |
| 204 | 1-15 | 0-10 | 10-15 | 80-90 | 0-35 or ≥25 | 0-7.5 | 0-7.5 |
| 205 | 1-15 | 0-5 | 0-10 | 85-95 | 0-35 or ≥25 | 0-7.5 | 0-7.5 |
| 206 | 1-15 | 0-10 | 5-10 | 85-95 | 0-35 or ≥25 | 0-7.5 | 0-7.5 |
| 207 | 1-15 | 0-10 | 0-10 | 25-95 | 0-35 or ≥25 | 0-7.5 | 0-7.5 |
| 208 | 1-15 | 0-10 | 10-45 | 25-95 | 0-35 or ≥25 | 0-7.5 | 0-7.5 |
| 209 | 1-15 | 0-10 | 45-90 | 25-95 | 0-35 or ≥25 | 0-7.5 | 0-7.5 |
| 210 | 1-15 | 0-10 | 0-10 | 25-75 | 0-35 or ≥25 | 0-7.5 | 0-7.5 |
| 211 | 1-15 | 0-10 | 10-35 | 25-75 | 0-35 or ≥25 | 0-7.5 | 0-7.5 |
| 212 | 1-15 | 0-10 | 35-70 | 25-75 | 0-35 or ≥25 | 0-7.5 | 0-7.5 |
| 213 | 1-15 | 0-10 | 0-10 | 25-55 | 0-35 or ≥25 | 0-7.5 | 0-7.5 |
| 214 | 1-15 | 0-10 | 10-25 | 25-55 | 0-35 or ≥25 | 0-7.5 | 0-7.5 |
| 215 | 1-15 | 0-10 | 25-35 | 25-55 | 0-35 or ≥25 | 0-7.5 | 0-7.5 |
| 216 | 1-15 | 0-10 | 35-55 | 25-55 | 0-35 or ≥25 | 0-7.5 | 0-7.5 |
| 217 | 1-15 | 0-5 | 5-10 | 50-75 | 0-35 or ≥25 | 0-7.5 | 0-7.5 |
| 218 | 1-15 | 0-10 | 10-20 | 50-75 | 0-35 or ≥25 | 0-7.5 | 0-7.5 |
| 219 | 1-15 | 0-10 | 20-35 | 50-75 | 0-35 or ≥25 | 0-7.5 | 0-7.5 |
| 220 | 1-15 | 0-10 | 35-75 | 50-75 | 0-35 or ≥25 | 0-7.5 | 0-7.5 |
| 221 | 1-15 | 0-5 | 5-10 | 75-95 | 0-35 or ≥25 | 0-7.5 | 0-7.5 |
| 222 | 1-15 | 0-10 | 10-20 | 75-95 | 0-35 or ≥25 | 0-7.5 | 0-7.5 |
| 223 | 15-25 | 0-5 | 0-5 | 50-75 | 0-35 or ≥25 | 0-7.5 | 0-7.5 |
| 224 | 15-25 | 0-10 | 0-10 | 50-75 | 0-35 or ≥25 | 0-7.5 | 0-7.5 |
| 225 | 15-25 | 5-10 | 0-5 | 50-75 | 0-35 or ≥25 | 0-7.5 | 0-7.5 |
| 226 | 15-25 | 0-5 | 5-10 | 50-75 | 0-35 or ≥25 | 0-7.5 | 0-7.5 |
| 227 | 15-25 | 5-10 | 5-10 | 50-75 | 0-35 or ≥25 | 0-7.5 | 0-7.5 |
| 228 | 15-25 | 10-20 | 0-10 | 50-75 | 0-35 or ≥25 | 0-7.5 | 0-7.5 |
| 229 | 15-25 | 0-10 | 10-20 | 50-75 | 0-35 or ≥25 | 0-7.5 | 0-7.5 |
| 230 | 15-25 | 20-35 | 0-10 | 50-75 | 0-35 or ≥25 | 0-7.5 | 0-7.5 |
| 231 | 15-25 | 0-10 | 20-35 | 50-75 | 0-35 or ≥25 | 0-7.5 | 0-7.5 |
| 232 | 15-25 | 0-5 | 0-5 | 75-95 | 0-35 or ≥25 | 0-7.5 | 0-7.5 |
| 233 | 15-25 | 0-10 | 0-10 | 75-95 | 0-35 or ≥25 | 0-7.5 | 0-7.5 |
| 234 | 15-25 | 5-10 | 0-5 | 75-95 | 0-35 or ≥25 | 0-7.5 | 0-7.5 |
| 235 | 15-25 | 0-5 | 5-10 | 75-95 | 0-35 or ≥25 | 0-7.5 | 0-7.5 |
| 236 | 1-15 | 64-80 | | 8-28 | 0-35 or ≥25 | 0-7.5 | 0-7.5 |
| 237 | 1-15 | | 64-80 | 8-28 | 0-35 or ≥25 | 0-7.5 | 0-7.5 |
| 238 | 8-12 | 68-76 | | 14-22 | 0-35 or ≥25 | 0-7.5 | 0-7.5 |
| 239 | 8-12 | | 68-76 | 14-22 | 0-35 or ≥25 | 0-7.5 | 0-7.5 |
| 240 | 9-11 | 70-74 | | 16-20 | 0-35 or ≥25 | 0-7.5 | 0-7.5 |
| 241 | 9-11 | | 70-74 | 16-20 | 0-35 or ≥25 | 0-7.5 | 0-7.5 |
| 242 | 9-11 | 71-73 | | 17-19 | 0-35 or ≥25 | 0-7.5 | 0-7.5 |
| 243 | 9-11 | | 71-73 | 17-19 | 0-35 or ≥25 | 0-7.5 | 0-7.5 |
| 244 | 10 | 72 | | 18 | 0-35 or ≥25 | 0-7.5 | 0-7.5 |
| 245 | 10 | | 72 | 18 | 0-35 or ≥25 | 0-7.5 | 0-7.5 |
| 246 | 1-15 | 10-60 | | 35-75 | 0-35 or ≥25 | 0-7.5 | 0-7.5 |
| 247 | 1-15 | | 10-60 | 35-75 | 0-35 or ≥25 | 0-7.5 | 0-7.5 |
| 248 | 1-15 | 35-60 | | 35-55 | 0-35 or ≥25 | 0-7.5 | 0-7.5 |
| 249 | 1-15 | | 35-60 | 35-55 | 0-35 or ≥25 | 0-7.5 | 0-7.5 |
| 250 | 1-15 | 15-35 | | 60-70 | 0-35 or ≥25 | 0-7.5 | 0-7.5 |
| 251 | 1-15 | | 15-35 | 60-70 | 0-35 or ≥25 | 0-7.5 | 0-7.5 |
| 252 | 1-15 | 0-25 | | 70-80 | 0-35 or ≥25 | 0-7.5 | 0-7.5 |
| 253 | 1-15 | | 0-25 | 70-80 | 0-35 or ≥25 | 0-7.5 | 0-7.5 |
| 254 | 1-15 | 0-15 | | 70-80 | 0-35 or ≥25 | 0-7.5 | 0-7.5 |
| 255 | 1-15 | | 0-15 | 70-80 | 0-35 or ≥25 | 0-7.5 | 0-7.5 |
| 256 | 1-15 | 0-15 | | 80-90 | 0-35 or ≥25 | 0-7.5 | 0-7.5 |
| 257 | 1-15 | | 0-15 | 80-90 | 0-35 or ≥25 | 0-7.5 | 0-7.5 |
| 258 | 1-15 | 0-10 | | 85-95 | 0-35 or ≥25 | 0-7.5 | 0-7.5 |
| 259 | 1-15 | | 0-10 | 85-95 | 0-35 or ≥25 | 0-7.5 | 0-7.5 |
| 260 | 1-15 | 0-5 | | 85-95 | 0-35 or ≥25 | 0-7.5 | 0-7.5 |
| 261 | 1-15 | | 0-5 | 85-95 | 0-35 or ≥25 | 0-7.5 | 0-7.5 |
| 262 | 1-15 | 0 | | 85-95 | 0-35 or ≥25 | 0-7.5 | 0-7.5 |
| 263 | 1-15 | | 0 | 85-95 | 0-35 or ≥25 | 0-7.5 | 0-7.5 |
| 264 | 1-15 | 0 | 0 | 85-95 | 0-35 or ≥25 | 0-7.5 | 0-7.5 |
| 265 | 1-15 | 0-16 | | 45-70 | 0-35 or ≥25 | 0-7.5 | 0-7.5 |
| 266 | 1-15 | 0-15 | | 46-70 | 0-35 or ≥25 | 0-7.5 | 0-7.5 |
| 267 | 1-15 | 0-14 | | 47-70 | 0-35 or ≥25 | 0-7.5 | 0-7.5 |
| 268 | 1-15 | 0-13 | | 48-70 | 0-35 or ≥25 | 0-7.5 | 0-7.5 |
| 269 | 1-15 | 0-12 | | 49-70 | 0-35 or ≥25 | 0-7.5 | 0-7.5 |
| 270 | 1-15 | 0-12 | | 51-70 | 0-35 or ≥25 | 0-7.5 | 0-7.5 |
| 271 | 1-15 | 0-11 | | 52-70 | 0-35 or ≥25 | 0-7.5 | 0-7.5 |
| 272 | 1-15 | 0-10 | | 52.5-70 | 0-35 or ≥25 | 0-7.5 | 0-7.5 |
| 273 | 1-15 | 0-8 | | 55-70 | 0-35 or ≥25 | 0-7.5 | 0-7.5 |

In further embodiments, at least one active ingredient of any one composition selected from 116-273 of Table 2 is a cannabinoid, cannabinoid extract, terpene, or terpene extract. In further embodiments, at least one active ingredient is a cannabinoid. In further embodiments, at least one active ingredient is a cannabinoid extract. In further embodiments, at least one active ingredient is a terpene. In further embodiments, at least one active ingredient is a terpene extract.

In further embodiments, a composition selected from one of the compositions from 116-273 of Table 2 is a non-aqueous composition.

In further embodiments, a composition selected from one of the compositions from 116-273 of Table 2 is a solid or semi-solid composition.

In another embodiment, a composition of Table 2, where permissible, comprises: 0.01-0.1 wt %, 0.1-1 wt %, 1 wt %, 0.5-1 wt %, 1-2.5 wt %, 1-3 wt %, 2.5-5 wt %, 3-8 wt %, 5.7.5 wt %, 5-10 wt %, 7.5-10 wt %, 7.5-15 wt %, 8-15 wt %, 8-12 wt %, 9-11 wt %, more than 8 wt %, more than 10 wt %, 10-12.5 wt %, 12.5-15 wt %, 10-15 wt %, 10-20 wt %, 20-30 wt %, 30-40 wt %, 40-50 wt %, or >50 wt % of an active ingredient(s), preferably comprising a cannabinoid or cannabinoid extract. In further embodiments, a composition selected from one of the compositions from 116-273 of Table 2 comprises: 8-15 wt %, 8-12 wt %, 9-11 wt %, more than 8 wt %, more than 10 wt %, or 10-15 wt % of at least one active ingredient, e.g., a cannabinoid or cannabinoid extract. In further embodiments, a composition selected from one of the compositions from 1-213, 227, 228, and 237-255 of Table 2 comprises 1-5 wt %, 3-8%, or 8-12 wt % of at least one active ingredient, e.g., a cannabinoid or cannabinoid extract.

In further embodiments, the cannabinoid extract comprises a cannabinoid(s) in an amount selected from: 50-75 wt %, 50-99 wt %, 75-99 wt %, 75-95 wt %, 80-99 wt %, 85-99 wt %, 90-99 wt %, 85-95 wt %, 90-95 wt %, or >99 wt % cannabinoids.

In further embodiments, the total concentration of the active ingredient(s), e.g., cannabinoid(s), in a composition selected from 116-273 of Table 2 is 1-200 mg/mL. In further embodiments, the total concentration of the active ingredient(s), e.g., cannabinoid(s), in a composition selected from 116-273 of Table 2 is selected from: 1-5 mg/mL, 1-10 mg/mL, 1-50 mg/mL, 1-100 mg/mL, 5-50 mg/mL, 10-50 mg/mL, 10-100 mg/mL, 5-10 mg/mL, 10-15 mg/mL, 15-20 mg/mL, 20-30 mg/mL, 30-40 mg/mL, 40-50 mg/mL, 50-75 mg/mL, 75-100 mg/mL, 100-150 mg/mL, or 150-200 mg/mL. In another embodiment, the total concentration of the active ingredient(s), e.g., cannabinoid(s), in a composition selected from one of the compositions from 116-273 of Table 2 is <0.001 mg/mL, 0.001-0.01 mg/mL, or 0.01-1 mg/mL.

In further embodiments, a composition selected from one of the compositions from 116-273 of Table 2 contains the active ingredient(s), e.g., cannabinoid(s), in an amount selected from: 0.25-1 mg, 0.5-2.5 mg, 2.5-5 mg, 5-7.5 mg, 7.5-10 mg, 10-12.5 mg, 12.5-15 mg, 15-20 mg, 20-30 mg, 30-40 mg, 40-50 mg, 50-60 mg, 60-70 mg, or 70-75 mg. In further embodiments, the cannabinoid is THC. In other embodiments, the cannabinoids are THC and CBD. In another embodiment, a composition selected from 116-273 of Table 2 comprises <0.001 mg, 0.001-0.25 mg, or 0.25-1 mg.

In further embodiments, the surfactant in a composition selected from compositions 116-273 of Table 2 is polysorbate 80, polysorbate 80 (polyoxyethylene (20) sorbitan monooleate, E433) and polyethylene glycol (PEG) 40 hydrogenated castor oil (Kolliphor RH40), or polyethylene glycol (PEG) 40 hydrogenated castor oil (Kolliphor RH40). In further embodiments, the surfactant in a composition selected from compositions 116-273 of Table 2 is polyoxyethylene (10) oleyl ether (e.g., BRIJ 010). In further embodiments, the surfactant in a composition selected from compositions 116-273 of Table 2 is macrogol 15 hydroxystearate (e.g., Solutol HS 15). In one embodiment, a composition of Table 2, where permissible, comprises a total amount of surfactant selected from: 0-2.5 wt %, 2.5-5 wt %, 5-10 wt %, 10-15 wt %, 15-20 wt %, 20-25 wt %, 25-30 wt %, 30-35 wt %, 35-40 wt %, 40-45 wt %, 45-50 wt %, 45-55 wt %, 45-65 wt %, 46-65 wt %, 47-65 wt %, 48-65 wt %, 49-65 wt %, 50-65 wt %, 51-65 wt %, 52-65 wt %, 53-65 wt %, 54-65 wt %, 55-65 wt %, 50-55 wt %, 55-60 wt %, 60-65 wt %, 65-70 wt %, 70-75 wt %, 75-80 wt %, 80-85 wt %, 85-90 wt %, 90-95 wt %, or 95-97 wt % surfactant.

In further embodiments, where permissible based on the ranges for a particular formula, a composition of Table 2 comprises no more than 25 wt %, 20 wt %, 15 wt %, 10 wt %, 5 wt %, 3 wt %, or 1 wt % MCT. In further embodiments, the MCT is an oil. In further embodiments, the composition comprises no MCT. In further embodiments, where permissible based on the ranges for a particular formula, a composition of Table 2 comprises no more than 25 wt %, 20 wt %, 15 wt %, 10 wt %, 5 wt %, 3 wt %, or 1 wt % LCT. In further embodiments, the LCT is an oil. In further embodiments, the composition comprises no LCT. In further embodiments, the composition comprises both MCT and LCT. In further embodiments, where permissible based on the ranges for a particular formula, a composition of Table 2 comprises no more than 25 wt %, 20 wt %, 15 wt %, 10 wt %, 5 wt %, 3 wt %, or 1 wt % combined MCT and LCT. In further embodiments, both the MCT and the LCT are an oil. In a further embodiment, a composition of Table 2, wherein permissible, comprises a total amount of combined MCT and LCT selected from: 0-5 wt %, 0-10 wt %, 0-11 wt %, 0-12 wt %, 0-13 wt %, 0-14 wt %, 0-15 wt %, 0-16 wt %, 0.5-1 wt %, 1-2 wt %, 1-2.5 wt %, 1-5 wt %, 1-10 wt %, 1-20 wt %, 2-3 wt %, 3-4 wt %, 4-5 wt %, 5-7.5 wt %, 5-10 wt %, 5-11 wt %, 5-12 wt %, 5-13 wt %, 5-14 wt %, 5-15 wt %, 5-16 wt %, 10-12.5 wt %, 10-15 wt %, 10-20 wt %, 15-20 wt %, or 20-25 wt %, 25-30 wt %, or 25-50% wt % combined MCT/LCT.

The medium chain triglycerides (MCT) of the present invention are triglycerides whose fatty acids have an aliphatic tail of 6-12 carbon atoms. The MCT may be a single MCT or a mix of MCT. In one embodiment, the MCT is formed from fatty acids having from C6 to C8, C8 to C10, C10 to C12, or C8 to C12 carbon atoms. The fatty acids of the MCT may be saturated, mono-unsaturated, and/or poly-unsaturated fatty acids. In one embodiment 80 to 100% of the medium chain fatty acids are saturated, 0 to 10% are monounsaturated, and 0 to 5% are polyunsaturated. Preferred medium chain fatty acids include caproic acid, caprylic acid, capric acid, and mixtures thereof. An oil comprising MCT, may comprise at least 5 wt % medium chain triglycerides, e.g., coconut oil, or palm kernel oil. In one embodiment, the oil comprising an MCT is coconut oil. MCT may be in the form of oil that is enriched or fractionated to increase the concentration of medium chain triglycerides. In one embodiment, the MCT is fractionated coconut oil (e.g., glyceryl tricaprylate or NATURE'S OIL MCT). Medium chain triglycerides may also be formed by esterifying glycerol with mixtures of C6-C12 fatty acids, e.g., C8-C10 fatty acids such as caprylic (C:8) and capric (C:10) fatty acids fractionated from coconut or palm kernel oils.

The long chain triglycerides (LCT) of the present invention are triglycerides whose fatty acids have an aliphatic tail of 13-24 carbon atoms. The LCT may be a single LCT or a mix of MCT. In one embodiment, the LCT is formed from long chain fatty having from C14 to C16, C16 to C18, C18 to C20, C14 to C20, or C20 to C24 carbon atoms. The fatty acids of the LCT may be saturated, mono-unsaturated, and poly-unsaturated fatty acids. In one embodiment 5 to 25% of the long chain fatty acids are saturated, 15 to 80% are monounsaturated, and 15 to 80% are polyunsaturated. The oil comprising an LCT may comprise at least 5 wt % long chain triglycerides, e.g., olive oil, poppy seed, safflower, sunflower, corn, and soybean oils, sesame oil, or castor oil. LCT may be in the form of oil that is enriched or fractionated to increase the concentration of long chain triglycerides. In one embodiment, the LCT is olive oil.

The oil comprising an MCT and/or LCT may be selected from the group consisting of borage oil, castor oil, coconut oil, cottonseed oil, soybean oil, safflower oil, sunflower oil, castor oil, corn oil, olive oil, palm oil, peanut oil, poppy seed oil, canola oil, hydrogenated soybean oil, hydrogenated vegetable oils, sesame oil, triolein, trilinolein, and trilinolenin.

The compositions of the present invention are preferably for oral administration.

As used herein, “emulsion” refers to a colloidal dispersion of two immiscible liquids, for example, an oil and water (or other aqueous medium, e.g., a polar solvent, simulated gastric fluid, gastric fluid, simulated intestinal fluid, intestinal fluid), one of which is part of a continuous phase and the other of which is part of a dispersed phase. Emulsions typically are stabilized by one or more surfactants and/or co-surfactants and/or emulsion stabilizers. Surfactants form an interfacial film between the oil and water phase of the emulsion, providing stability. Typically, emulsions contain micelles that contain one or more surfactants surrounding a non-polar compound which is dispersed in the water phase. In general, emulsions (e.g., oil-in-water emulsions) are colloidal dispersions of two immiscible liquids (e.g., an oil and an aqueous medium, such as water) that contain a continuous and a dispersed phase. Emulsions can be used to disperse non-polar compounds in aqueous media. In an oil-in-water emulsion, the dispersed phase is an oil phase and the continuous phase is an aqueous (e.g., water) phase. Some of the compositions of the present invention self-emulsify in aqueous media, e.g., water, gastric fluids or intestinal fluids, to form an oil-in-water emulsion.

As used herein, “surfactant” refers to synthetic and naturally occurring amphiphilic molecules that have hydrophobic portion(s) and hydrophilic portion(s). Due to their amphiphilic (amphipathic) nature, surfactants typically can reduce the surface tension between two immiscible liquids, for example, the oil and water phases in an emulsion, stabilizing the emulsion. Surfactants can be characterized based on their relative hydrophobicity and/or hydrophilicity. For example, relatively lipophilic surfactants are more soluble in fats, oils and waxes, and typically have HLB values less than or about 10, while relatively hydrophilic surfactants are more soluble in aqueous compositions, for example, water, and typically have HLB values greater than or about 10. Relatively amphiphilic surfactants are soluble in oil- and water-based liquids and typically have HLB values close to 10 or about 10.

The “HLB” refers to a value that is used to index and describe a surfactant according to its relative hydrophobicity/hydrophilicity, relative to other surfactants. HLB number of a surfactant is defined as HLB=20*MH/MT, where MH and MT are the mass of the hydrophilic head group and the total surfactant mass, respectively. A surfactant's HLB value is an indication of the molecular balance of the hydrophobic and hydrophilic portions of the surfactant, which is an amphipathic molecule.

As used herein, “micelle” refers to aggregates formed by surfactants that typically form when a surfactant is present in an aqueous composition, typically when the surfactant is used at a concentration above the critical micelle concentration (CMC). In micelles, the hydrophilic portions of the surfactant molecules contact the aqueous or the water phase, while the hydrophobic portions form the core of the micelle, which can encapsulate non-polar ingredient(s), for example, a cannabinoid.

In one embodiment, the composition of the present invention is self-emulsifying in an aqueous medium. In a further embodiment, the composition forms a micellar dispersion in an aqueous medium.

In another embodiment, the composition of the present invention further comprises an aqueous medium. In a further embodiment, the aqueous medium is selected from a polar solvent, water, simulated gastric fluid, gastric fluid, simulated intestinal fluid, or intestinal fluid. In another embodiment, the surfactant is at a concentration that is greater than its critical micelle concentration (CMC). In one embodiment, the composition is a micellar dispersion. In another embodiment, the composition is an emulsion. In a further embodiment, the emulsion is an oil-in-water emulsion.

In another embodiment, the invention provides for a beverage additive product comprising a composition of the present invention. For example, a beverage additive composition can contain one or more active ingredients, e.g., an active ingredient(s) derived from a cannabis plant, such as, one or more cannabinoid(s), terpene(s) or any other active ingredient of cannabis plant extract. The active ingredient(s) of the beverage additive can also be one or more cannabinoid(s), terpene(s) or any other active ingredient of cannabis plant extract that is/are derived synthetically. In one embodiment, the beverage additive comprises CBD, ethyl pyruvate, THC, beta-caryophyllene, linalool, limonene, alpha-pinene, and eucalyptol. In addition to a surfactant, an optionally an oil, the beverage additive may further contain a flavoring or flavoring agent(s), sweetener, or an edible carrier. The beverage additive may be provided in liquid, semi-solid, or solid form.

The concentration of each active ingredient, independently, or total active ingredients (e.g., CBD and ethyl pyruvate) in the beverage additive may be selected from: <0.001 mg/mL, 0.001-0.01 mg/mL, or 0.01-1 mg/mL, 1-5 mg/mL, 1-10 mg/mL, 1-50 mg/mL, 1-100 mg/mL, 5-50 mg/mL, 10-50 mg/mL, 10-100 mg/mL, 5-10 mg/mL, 10-15 mg/mL, 15-20 mg/mL, 20-30 mg/mL, 30-40 mg/mL, 40-50 mg/mL, 50-75 mg/mL, 75-100 mg/mL, 100-150 mg/mL, 150-200 mg/mL, 200-250 mg/mL, 250-300 mg/mL, 300-350 mg/mL, 350-400 mg/mL, 400-450 mg/mL, 450-500 mg/mL, 500-550 mg/mL, 100-200 mg/mL, 200-300 mg/mL, 30-400 mg/mL, 400-500 mg/mL, 600-700 mg/mL, 700-800 mg/mL, 800-900 mg/mL, or >900 mg/mL.

In one embodiment, the concentration of total active ingredients, e.g., cannabinoids, in the beverage additive is selected from <0.001 mg/mL, 0.001-0.01 mg/mL, or 0.01-1 mg/mL, 1-5 mg/mL, 1-10 mg/mL, 1-25 mg/mL, 25-50 mg/mL, 25-75 mg/mL, 1-50 mg/mL, 1-100 mg/mL, 5-50 mg/mL, 10-50 mg/mL, 10-100 mg/mL, 5-10 mg/mL, 10-15 mg/mL, 15-20 mg/mL, 20-30 mg/mL, 30-40 mg/mL, 40-50 mg/mL, 50-75 mg/mL, 75-100 mg/mL, 100-150 mg/mL, or 150-200 mg/mL.

In one embodiment, the amount for each cannabinoid, independently, or total active cannabinoids in a dose/serving of the beverage additive is selected from: <0.001 mg, 0.001-0.25 mg, or 0.25-1 mg, 0.25-1 mg, 0.5-2.5 mg, 2.5-5 mg, 5-7.5 mg, 7.5-10 mg, 10-12.5 mg, 12.5-15 mg, 15-20 mg, 20-30 mg, 30-40 mg, 40-50 mg, 50-60 mg, 60-70 mg, 70-75 mg, 75-100 mg, 100-150 mg, 150-200 mg, or >200 mg.

In another embodiment, the total ethyl pyruvate in a dose/serving of the beverage additive is selected from: 50-75 mg, 75-100 mg, 100-150 mg, 150-200 mg, 200-250 mg, 250-300 mg, 300-350 mg, 350-400 mg, 400-450 mg, 450-500 mg, 500-550 mg, 100-200 mg, 200-300 mg, 30-400 mg, 400-500 mg, 600-700 mg, 700-800 mg, 800-900 mg, 900-1000 mg, 1.0-2.0 g, 2.0-3.0 g, 3.0-4.0 g, 4.0-5.0 g, 5.0-6.0 g, 6.0-7.0 g, 7.0-8.0 g, 8.0-9.0 g, 9.0-10 g, 10-11 g, 11-12 g, 12-12.5, or >12.5 g.

Prior to ingestion, the beverage additive can be added to water or any drink of choice. The dilution ratio of beverage additive:beverage will depend on the composition of the beverage additive and selection of beverage type. In one embodiment, the beverage additive is diluted from 1:1-10,000 (i.e., 1 part beverage additive to 1-10,000 parts beverage). In further embodiments, the ratio is 1:1,000-10,000, 1:750-1,000, 1:500-750, 1:250-500, 1:100-250, 1:75-100, 1:50-75, 1:25-50, 1:10-25, 1:7.5-10, 1:5-7.5, 1:2.5-5, 1:1-2.5, or 1:1. In another embodiment the ratio beverage additive to beverage is 1:0.5-1. In one embodiment, the beverage additive is added to a beverage to provide an aqueous emulsion. In one embodiment, the aqueous emulsion is transparent.

Depending on the composition, aqueous emulsification may require mechanical input or agitation, such as shaking, mixing or stirring. Depending on the composition, the organoleptic properties of the emulsion may vary. For example, high surfactant content beverage additives can form clear, transparent emulsions, while compositions containing oils can form more turbid, i.e., translucent or opaque emulsions. In one embodiment, the composition is a rapid dispersing formulation, forming a transparent emulsion (nanoemulsion or microemulsion) or micellar dispersion within a time selected from: 5, 10, 15, 20, 25, 30, 35, 40, 45, 50, 55 or 60, or 90 seconds, or within 2, 2.5, 3, 3.5, 4, 4.5, or 5 minutes after addition to an aqueous medium at a temperature selected from: 4 degrees, 20 degrees, 40 degrees, or 60 degrees C., wherein the final concentration of the composition in the aqueous medium is 0.1 wt %. In a further embodiment, the composition, form a transparent dispersion or emulsion within 3 min, 90 sec or 60 sec at 20 degrees C. In the further embodiment, the rapid dispersing formulation does not require agitation (e.g., shaking or stirring) to form a transparent emulsion within a time selected from: 5, 10, 15, 20, 25, 30, 35, 40, 45, 50, 55 or 60, or 90 seconds, or within 2, 2.5, 3, 3.5, 4, 4.5, or 5 minutes after addition to an aqueous medium at a temperature selected from: 4 degrees, 20 degrees, 40 degrees, or 60 degrees C., wherein the final concentration of the composition in the aqueous medium is 0.1 wt %. In one embodiment, said emulsion or micellar dispersion is a thermodynamically stable, isotropic liquid.

The taste or flavor of the emulsion can vary with the composition, such as the exact content of active ingredient(s), surfactant(s), oil(s), flavoring agent(s), sweetener(s) and edible carrier(s). Due to high “solvent capacity” or “dilutability” of some compositions presented in this invention, the emulsion can retain its desirable particle size distribution upon ingestion and dilution in the gut. This can provide pharmacokinetic benefits, such as faster onset of action, increased bioavailability and reduced pharmacokinetic variability, e.g., reduced dependence of pharmacokinetics on digestion, and reduced food effects.

The beverage additive may be added to any beverage suitable for human consumption. Examples include, water, milk, tea, coffee, fruit juice (e.g., orange, apple, cranberry, pear, currant, etc.), vegetable juice (e.g., carrot, tomato, etc.), and carbonated drinks (etc. sparkling water, soda water, sports drinks, and soft drinks such as colas). In one embodiment, the invention includes a combination of a beverage additive and a beverage or a kit comprising the beverage additive and the beverage, wherein the beverage additive and the beverage are in separate containers. In another embodiment, the beverage additive and the beverage are separate compartments of a container. For example, where the beverage additive is contained in a compartment in a cap/closure of a container. In another embodiment, the invention provides for a method of making a cannabis plant-based beverage comprising a composition of the present invention, the method comprising the steps of: obtaining a beverage additive and a beverage; adding the beverage additive to the beverage; and mixing the combined beverage additive and beverage to form a cannabis plant based beverage. In a further embodiment, the combined beverage is homogeneous. In a further embodiment, the combined beverage is an emulsion.

In another embodiment, the invention provides for a beverage comprising the beverage additive. In some embodiments, the beverage is an aqueous beverage. In further embodiments, the aqueous beverage is selected from water, coffee, tea, fruit juice (e.g., orange, apple, cranberry, pear, pineapple, currant, etc.), algae (e.g., blue-green algae), vegetable juice (e.g., carrot, tomato, wheat or other grass, mixed vegetable or mixed vegetable-fruit etc.), sports drinks, and carbonated drinks (etc. sparkling water, soda water, and soft drinks such as colas). In other embodiments, the beverage is a dairy based beverage. In further embodiments, the dairy based beverage is selected from milk and yogurt drinks (including beverages that comprise milk or yogurt).

In one embodiment, the invention relates to a drinking straw for use with a beverage in a beverage container, wherein the drinking straw comprises a composition (e.g., cannabinoid composition) of the present invention (including a beverage additive). In some embodiments, the drinking straw comprises a compartment or an erodible surface within an interior portion of the straw that contains the composition of the present invention, e.g., cannabinoid composition. The straw may further comprise a one-way valve that prevents the composition of the present invention, e.g., cannabinoid composition from entering the beverage container. Examples of drinking straws of include those disclosed in United States patents U.S. Pat. Nos. 5,921,955, 8,342,422, 6,482,451, and 8,980,348; United States patent applications US 2012/0056008, US 2008/0181932, US 2004/0142958, and US 2009/0041904; and in PCT publication WO 2001/014220.

The term “particle size” refers herein to oil in water droplet diameter, or water in oil droplet diameter, in an emulsion. The average particle size of the emulsion is in the range of about 50 nm to about 1000 nm, depending on the composition. In one embodiment, the average particle size is between 10-50 nm. In another embodiment, the average particle size is between 50-100 nm. In another embodiment, the average particle size is between 75-125 nm. In another embodiment, the average particle size is between 100-150 nm. In another embodiment, the average particle size is between 200-400 nm. In another embodiment, the average particle size is between 200-300 nm. In another embodiment, the average particle size is between 250-350 nm. In another embodiment, the average particle size is between 300-400 nm. In another embodiment, the average particle size is between 400-500 nm. In another embodiment, the average particle size is between 500-600 nm. In another embodiment, the average particle size is between 600-650 nm. In another embodiment, the average particle size is between 600-700 nm. In another embodiment, the average particle size is between 700-800 nm. In another embodiment, the average particle size is between 800-900 nm. In another embodiment, the average particle size is between 750-850 nm. In one embodiment, the average particle size is less than 500 nm. In another embodiment, the average particle size is less than 400 nm. In another embodiment, the average particle size is less than 300 nm. In another embodiment, the average particle size is less than 200 nm. In another embodiment, the average particle size is less than 150 nm. In another embodiment, the average particle size is less than 100 nm. In another embodiment, the average particle size is less than 50 nm.

The term “chemically stable” or “chemical stability” of a composition of the present invention refers to the ability of the composition and/or cannabinoid(s) in the composition to resist change in its chemical properties over time. Chemical instability of a composition may be manifested by decrease in the amount of the active ingredient, e.g., cannabinoid, e.g., THC or CBD. Chemical degradation of THC, e.g., may occur due to conversion of TCH to cannabinol (CBN). Chemical degradation of CBD, e.g., may occur due to oxidation, resulting in monomeric and dimeric hydroxyquinones. Physical instability of an emulsion may be manifested in any of the following: flocculation, creaming, coalescence and Ostwald ripening. Determination whether an emulsion has lost its physical stability may be carried out in any of the following techniques: measurement of particle size, light scattering, focused beam reflectance measurement, centrifugation, rheology or a combination thereof.

In one embodiment, the composition is stable at room temperature (21-25° C.), for about 12 months at 25° C.±2° C./40% RH ±5% RH, with <20% decrease, <10% decrease preferably <5% decrease, in active ingredient content, e.g., in cannabinoid content, e.g., total, THC or CBD, and no change on dispersion in 37° C. water over the 12 months. It is also an object of the present invention to provide the composition as mentioned above, wherein the composition is stable at 5° C.±3° C./40% RH±5% RH for about 6 months, preferably 12 months, more preferably about 24 months, with <20% decrease, <10% decrease, preferably <5% decrease, in active ingredient, e.g., in cannabinoid content, e.g., total, THC or CBD, and no change on dispersion in 37° C. water over the relevant time frame. It is also an object of the present invention to provide the composition as mentioned above, wherein the composition is stable at about 40° C.±2° C./75% RH±5% RH for about 2 months, preferably about 6 months, with <20% decrease, <10% decrease, preferably <5% decrease, in active ingredient, e.g., in cannabinoid content and no change on dispersion in 37° C. water over the relevant time frame.

Active ingredients of the present invention, e.g., cannabinoids and terpenes, may be purchased, synthesized using well-known techniques, or extracted from a plant using well-known methods. Terpenes, e.g., may be extracted from a plant of the Cannabis genus, e.g., Cannabis sativa, Cannabis indica, Cannabis hybrid, or other, or from a plant that is not a member of the Cannabis genus, e.g., is not from Cannabis sativa, Cannabis indica, Cannabis hybrid, or other Cannabis species. Phytocannabinoids and terpenes may be extracted as terpene blends or, in the case of a Cannabis species, as a cannabinoid or cannabinoid/terpene blend. The blends may be used directly or can be separated into individual or fewer components using distillation (e.g., short-path rotary distillation) or other techniques. The relative amount of each principal phytocannabinoid and/or terpene in the plant extract, e.g., cannabis extract, varies according to the cannabinoid and/or terpene profile and levels of the particular plants and methodology of extraction. Extracts comprising terpenes, e.g., extracts essentially free of cannabinoids, extracts that contain cannabinoids as a minor constituent, or extracts from a plant that is not a species of Cannabis (e.g., Cannabis sativa, Cannabis indica, Cannabis hybrid, or other), i.e., a non-Cannabis species, may be used individually or combined with one or more other active ingredients, e.g., cannabinoids or cannabinoid extracts.

Cannabinoids and/or terpenes may be obtained by separating resins from leaves or leaves and flowers of cannabis plants by solvent extraction. Extracts derived from cannabis plants include primary extracts prepared by such processes as, for example, maceration, percolation, and solvent extraction. Solvent extraction may be carried out using a solvent that dissolves cannabinoids/cannabinoid acids, such as for example C1 to C5 alcohols (e.g. ethanol, methanol), C3-C12 alkanes (e.g. hexane, butane or propane), Norflurane (HFA134a), HFA227, and carbon dioxide. General protocols for the preparation of extracts of cannabis plant material are described in US20060167283 (WO 02/064109), which is incorporated herein by reference. Carbon dioxide provides another method to extract cannabinoid/terpene resins from cannabis plant material. Sub Critical (Liquid) or Supercritical CO₂ is forced through the plant matter, which separates the cannabinoid/terpenes from the plant matter resulting in a transparent, amber oil. The extracts obtained by supercritical fluid extraction (SFE) may undergo a secondary extraction, e.g. an ethanolic precipitation, to remove non-cannabinoid/terpene materials. In a preferred embodiment, light petroleum gas extraction, using a LHBES (light hydrocarbon butane extraction system) 1300/C from Extractiontek Solutions is used to extract cannabinoids from cannabis plant material.

A modified extraction process consists of decarboxylating the starting concentrate at 300° F. until fully converted and the bubbling stops. Once the oil is decarboxylated, it is run through the VTA-VKL 70-5 short path rotary distillation plant twice. The first run separates the heavy terpenes and lighter terpenes from the cannabinoids and waste material. The cannabinoids and waste are run through again with a higher vacuum and higher temperature to separate the cannabinoids from the remaining waste. The waste is collected and run again in a larger batch to extract all cannabinoids and terpenes. The VTA-VKL 70-5 short path rotary distillation plant uses a top stirring rotary column to wipe incoming product into a thin film for better heat distribution and evaporation. The inner condensing column is set to condense the cannabinoids into liquids. The waste and cannabinoids are diverted into the two dispensing arms for collection into receiving vessels. The light terpenes are collected in a receiving flask attached to the inline chiller on the plant. The system (except for feed vessel) are under vacuum during the operation. The vacuum for the first run should be between 0.5-0.7 mbar. For the second run, pressure should be between 0.5-0.07 mbar.

The compositions and methods using of the present invention include a cannabinoid selected from the group consisting: of tetrahydrocannabinol, Δ9-tetrahydrocannabinol (THC), Δ8-tetrahydrocannabinol, a cannabis extract, tetrahydrocannabinolic acid (THCA), cannabigerolic acid (CBGA), cannabidiolic acid (CBDA), cannabinolic acid (CBNA), Δ8-tetrahydrocannabinol-DMH, Δ9-tetrahydrocannabinol propyl analogue (THCV), 11-hydroxy-tetrahydrocannabinol, 11-nor-9-carboxy-tetrahydrocannabinol, 5′-azido-Δ8-tetrahydrocannabinol, AMG-1, AMG-3, AM411, AM708, AM836, AM855, AM919, AM926, AM938, cannabidiol (CBD), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabidiol propyl analogue (CBDV), cannabinol (CBN), cannabichromene (CBC), cannabichromene propyl analogue, cannabigerol (CBG), cannabicyclol (CBL), cannabielsoin (CBE), cannabinodiol (CBDL), and cannabitriol (CBTL), CP 47497, CP 55940, CP 55244, CP 50556, CT-3 or IP-751 (ajulemic acid), dimethylheptyl HHC, HU-210, HU-211, HU-308, WIN 55212-2, desacetyl-L-nantradol, dexanabinol, JWH-051, JWH-133, levonantradol, L-759633, nabilone, O-1184, cannabicyclohexanol (CP-47,497 C8 homolog), 10-hydroxycannabidiol, 1′,2′,3′,4′,5′-pentanorcannabinol-3-carboxylic acid, 1′-hydroxycannabinol, 11-hydroxycannabinol, 9-carboxy-11-norcannabinol, 1′-oxocannabinol, 11-nor-Δ8-THC-9-carboxylic acid, 2′-carboxy-3′,4′,5′-trinor-Δ9-THC, 5′-carboxy-Δ9-THC, 9-carboxy-11-nor-Δ9-THC, 9-carboxy-11-nor-Δ8-THC, [(6aR,10aR)-3-[(1S,2R)-1,2-dimethylheptyl]-6a,7,10,10a-tetrahydro-6, 6,9-trimethyl-6H-dibenzo[b,d]pyran-1-ol], 9-carboxy-11-nor-(2 or 4)-chloro-Δ8-THC, 8α-11-dihydroxy-Δ9-THC, 8β-11-Dihydroxy-Δ9-THC, 5′-Dimethylamino-Δ8-THC, 11-hydroxy-Δ9-THC, 1′-hydroxy-Δ9-THC (Isomer B), 11-hydroxy-Δ8-THC, 2′-hydroxy-Δ9-THC, 3′-hydroxy-Δ9-THC, 4′-hydroxy-Δ9-THC, 5′-hydroxy-Δ9-THC, 8α-hydroxy-Δ9-THC, 8β-hydroxy-Δ9-THC, 5′-methylamino-Δ8-THC, 5′-N-methyl-N-4-(7-nitrobenzofurazano)amino-Δ8-THC, (−)-trans-Δ8-THC, 5′-trimethylammonium-Δ8-THC phenolate, 5′-Trimethylammonium-11-hydroxy-Δ8-THC phenolate, or a mixture thereof. In one embodiment, the composition comprises any one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, or more of the above cannabinoids. In a preferred embodiment, the cannabinoid is selected from the group consisting of THC, CBD, THCA, CBDA, CBV, THCV, CBDV, CBCV, CBGV, CBN, CBC, and CBDL. In another embodiment, the cannabinoid is selected from the group consisting of THC, CBD, THCA, and CBDA. In another embodiment, the cannabinoid is THC or CBD. In another embodiment, the THC is Δ9-THC or Δ8-THC. In another embodiment, the THC is Δ9-THC. In another embodiment, the THC is Δ8-THC.

In a preferred embodiment, the cannabinoid is in the form of a Cannabis sativa, Cannabis indica, or Cannabis hybrid extract. In one embodiment, the cannabis extract comprises 49 THC. In another embodiment, the extract comprises CBD. In another embodiment, the cannabinoid is a synthetic cannabinoid, e.g., dronabinol.

In one embodiment, a composition of the present invention comprises: 1-5 wt %, 5-10 wt %, more than 5 wt %, 8-15 wt %, 8-12 wt %, more than 8 wt %, 9-11 wt %, more than 10 wt %, 10-15 wt %, 15-20 wt %, 20-30 wt %, 30-40 wt %, 40-50 wt %, of a cannabinoid or cannabinoid extract.

In one embodiment, the cannabinoid extract comprises 50-99 wt % cannabinoids. In another embodiment, the cannabinoid extract comprises >99 wt % total cannabinoids. In another embodiment, the cannabinoid extract comprises a total amount of cannabinoid(s) selected from: 50-75 wt %, 50-99 wt %, 75-99 wt %, 75-95 wt %, 80-99 wt %, 85-99 wt %, 90-99 wt %, 85-95 wt %, or 90-95 wt % cannabinoids.

In one embodiment, the total concentration of cannabinoid(s) in a composition of the present invention is 1-200 mg/mL. In further embodiments, the total concentration of cannabinoid(s) in a composition of the present invention is selected from: 1-5 mg/mL, 1-10 mg/mL, 1-50 mg/mL, 1-100 mg/mL, 5-50 mg/mL, 10-50 mg/mL, 10-100 mg/mL, 5-10 mg/mL, 10-15 mg/mL, 15-20 mg/mL, 20-30 mg/mL, 30-40 mg/mL, 40-50 mg/mL, 50-75 mg/mL, 75-100 mg/mL, 100-150 mg/mL, or 150-200 mg/mL. In another embodiment, the total concentration of cannabinoid(s) in a composition of the present invention is <0.001 mg/mL, 0.001-0.01 mg/mL, or 0.01-1 mg/mL.

In one embodiment, the concentration of each cannabinoid(s), e.g., 49 THC, in a composition of the present invention is selected from: 1-5 mg/mL, 1-10 mg/mL, 1-50 mg/mL, 1-100 mg/mL, 5-50 mg/mL, 10-50 mg/mL, 10-100 mg/mL, 5-10 mg/mL, 10-15 mg/mL, 15-20 mg/mL, 20-30 mg/mL, 30-40 mg/mL, 40-50 mg/mL, 50-75 mg/mL, 75-100 mg/mL, 100-150 mg/mL, or 150-200 mg/mL. In a further embodiment, the cannabinoid(s) is selected from one or more of CBD, THC, THCA, CBDA, CBV, THCV, CBDV, CBCV, CBGV, CBN, CBC, or CBDL.

In a further embodiment, the concentration of at least one cannabinoid, e.g., CBD, is selected from 0.1-25 mg/mL, 25-50 mg/mL, 40-80 mg/mL, 80-120 mg/mL, or >120 mg/ml. In a further embodiment, the concentration of at least one cannabinoid, e.g., THC, is selected from 0.1-1.0 mg/ml, 1.0-2.5 mg/ml, 2.5-5.0 mg/mL, 5.0-10 mg/mL, 10-25 mg/mL, or 25-50 mg/mL, 50-75 mg/mL, 75-100 mg/mL, or >100 mg/ml.

In one embodiment, a composition of the present invention comprises: 1-5 wt %, 5-10 wt %, more than 5 wt %, 8-15 wt %, 8-12 wt %, more than 8 wt %, 9-11 wt %, more than 10 wt %, 10-15 wt %, 15-20 wt %, 20-30 wt %, 30-40 wt %, 40-50 wt %, of a CBD or CBD extract (i.e., a cannabinoid extract comprising CBD). In one embodiment, the CBD extract comprises 50-99 wt % CBD. In another embodiment, the CBD extract comprises >99 wt % total CBD. In another embodiment, the CBD extract comprises a total amount of CBD selected from: 50-75 wt %, 50-99 wt %, 75-99 wt %, 75-95 wt %, 80-99 wt %, 85-99 wt %, 90-99 wt %, 85-95 wt %, or 90-95 wt % CBD.

In one embodiment, a composition of the present invention comprises: 1-5 wt %, 5-10 wt %, more than 5 wt %, 8-15 wt %, 8-12 wt %, more than 8 wt %, 9-11 wt %, more than 10 wt %, 10-15 wt %, 15-20 wt %, 20-30 wt %, 30-40 wt %, 40-50 wt %, of a THC or THC extract (i.e., an extract comprising THC). In one embodiment, the THC extract comprises 50-99 wt % THC. In another embodiment, the THC extract comprises >99 wt % total THC. In another embodiment, the THC extract comprises a total amount of THC selected from: 50-75 wt %, 50-99 wt %, 75-99 wt %, 75-95 wt %, 80-99 wt %, 85-99 wt %, 90-99 wt %, 85-95 wt %, or 90-95 wt % THC.

The present invention includes at one terpene selected from the group consisting of: alpha-pinene, valencene, myrcene, camphene, beta-pinene, citral, humulene, alpha-bisabolol, beta-caryophyllene, camphor, limonene, linalool, alpha-phellandrene, eucalyptol, terpineol, nerolidol, y-terpinene, terpinolele, gama-3-carene, pulegone, geraniol, ocimene, eugenol, p-cymene, ocimene, isopulegol, geranyl acetate, valencene, myrcene, cadinene, cedrane, citronellol, guaiene, dextro carvone, carvacrol, borneol, thymol, menthol, phytol, dextro fenchone, caryophyllene acetate, caryophyllene oxide, farnesene, terpinolene, nerol and combinations thereof.

In one embodiment, the composition of the present invention comprises 0-50 wt % total terpene(s). In further embodiments, a composition of the present invention comprises a total amount of terpene(s) selected from: 0-0.1 wt %, 0-0.5 wt %, 0.5-1 wt %, 0-1 wt %, 0-5 wt %, 0-10 wt %, 0-25 wt %, 1-2 wt %, 2-3 wt %, 3-4 wt %, 4-5 wt %, 5-7.5 wt %, 5-10 wt %, 10-12.5 wt %, 10-15 wt %, 15-20 wt %, or 20-25 wt %, or 25-50% wt % terpene(s).

In another embodiment, the cannabinoid extract comprises a total amount of cannabinoid(s) and a total amount of terpene(s) selected from: 50-75 wt %, 50-99 wt %, 75-99 wt %, 75-95 wt %, 80-99 wt %, 85-99 wt %, 90-99 wt %, 85-95 wt %, 90-95 wt %, or >99 wt % cannabinoid(s); and 0-0.1 wt %, 0-0.5 wt %, 0.5-1 wt %, 0-1 wt %, 0-5 wt %, 0-10 wt %, 0-25 wt %, 1-2 wt %, 2-3 wt %, 3-4 wt %, 4-5 wt %, 5-7.5 wt %, 5-10 wt %, 10-12.5 wt %, 10-15 wt %, 15-20 wt %, or 20-25 wt %, or 25-50 wt % terpene(s).

In one embodiment, the terpenes and cannabinoids are co-extracted, i.e., extracted together. In another embodiment, some or all of the terpenes are extracted separately from the cannabinoids. In another embodiment, some or all of the terpenes are synthetic. In one embodiment, the total concentration of the terpene(s) in a composition of the present invention is selected from: 0.05-50 mg/mL, 0.05-0.1 mg/mL, 0.1-0.5 mg/mL, 0.5-1 mg/mL, 1-5 mg/mL, 5-10 mg/mL, 10-20 mg/mL, 20-30 mg/mL, 30-40 mg/mL, 40-50 mg/mL, 1-50 mg/mL, or 10-50 mg/mL.

In one embodiment, the total concentration of the terpene(s) in a composition of the present invention is selected from: <0.001 mg/mL, 0.001-0.01 mg/mL, or 0.01-1 mg/mL, 0.05-50 mg/mL, 0.05-0.1 mg/mL, 0.1-0.5 mg/mL, 0.5-1 mg/mL, 1-5 mg/mL, 1-10 mg/mL, 1-50 mg/mL, 1-100 mg/mL, 5-50 mg/mL, 10-50 mg/mL, 10-100 mg/mL, 5-10 mg/mL, 10-15 mg/mL, 15-20 mg/mL, 20-30 mg/mL, 30-40 mg/mL, 40-50 mg/mL, 40-60 mg/mL, 50-75 mg/mL, 75-100 mg/mL, 100-150 mg/mL, or 150-200 mg/mL, or >200 mg/ml.

In one embodiment, the total concentration of the terpene(s) in a composition of the present invention is selected from: 0.05-50 mg/mL, 0.05-0.1 mg/mL, 0.1-0.5 mg/mL, 0.5-1 mg/mL, 1-5 mg/mL, 5-10 mg/mL, 10-20 mg/mL, 20-30 mg/mL, 30-40 mg/mL, 40-50 mg/mL, 1-50 mg/mL, or 10-50 mg/mL.

In another embodiment, the terpene in selected from any one, two, three, four, five, or all six of the terpenes selected from the group consisting of: myrcene beta-caryophyllene, linalool, limonene, alpha-pinene, and eucalyptol. In a further embodiment, said terpenes include any one, two, three, four, or all five terpenes selected from the group consisting of: beta-caryophyllene, linalool, limonene, alpha-pinene, and eucalyptol. In a further embodiment, said formulation comprises 0-2.0% any one, two, three, four, or all five terpenes selected from the group consisting of: beta-caryophyllene, linalool, limonene, alpha-pinene, and eucalyptol. In a further embodiment, said formulation comprises 0.3-0.5% any one, two, three, four, or all five terpenes selected from the group consisting of: beta-caryophyllene, linalool, limonene, alpha-pinene, and eucalyptol. In a further embodiment, said formulation comprises 0.3-0.5% of each of the following terpenes: beta-caryophyllene, linalool, limonene, alpha-pinene, and eucalyptol. In a further embodiment, said formulation comprises: 0.25-0.45% beta-caryophyllene, 0-0.15% linalool, 0.5-1.0% limonene, 0.75-1.5% alpha-pinene, and 0-0.05% eucalyptol.

In one embodiment, said composition is a formulation selected from any one of A1-A34 or any one of BA9-BA25 (see Examples).

In one embodiment, the composition comprises: 0-15% THC, 0-20% CBD, 0-50% ethyl pyruvate, 0-10% terpenes. In another embodiment, the composition comprises: 0-10% THC, 0-15% CBD, 0-50% ethyl pyruvate, 0-10% terpenes. In another embodiment, the composition comprises: 0-5% THC, 0-10% CBD, 0-40% ethyl pyruvate, 0-5% terpenes. In another embodiment, the composition comprises: 0.05-5% THC, 0.5-10% CBD, 1-40% ethyl pyruvate, 0-5% terpenes. In another embodiment, the composition comprises: 0.05-1% THC, 2.5-10% CBD, 1-40% ethyl pyruvate, 0-5% terpenes. In another embodiment, the composition comprises: 0.1-0.5% THC, 4-7.5% CBD, 1.0-10 or 10-30% ethyl pyruvate, 1-2.5% terpenes. In another embodiment, said composition comprises 50-90% polysorbate 80. In another embodiment, said composition comprises 0-5% sweetener, e.g., sucralose.

In another embodiment, said composition comprises: 0-7.0% THC, 0-7.0% CBD, 9-30% ethyl pyruvate, 1-5% terpenes, at least 59% polysorbate 80, and 0-5.0% sucralose. In another embodiment, said composition comprises 0.25-1.0% THC, 4-9% CBD, 25-35% ethyl pyruvate, 1-3% terpenes, 0-5% sucralose, and 50-70% polysorbate 80. In another embodiment, said composition comprises 0.25-0.75% THC, 4-9% CBD, 7.5-12.5% ethyl pyruvate, 1-3% terpenes, 0-5% sucralose, and 70-85% polysorbate 80. In another embodiment, said composition comprises 0-0.25% THC, 3.5-9% CBD, 0.25-2.5% ethyl pyruvate, 1-3% terpenes, 0-5% sucralose, and 81.5-95.5% polysorbate 80. In another embodiment, said composition comprises 0.4-0.6% THC, 5.5-7.5% CBD, 7.5-12.5% ethyl pyruvate, 1-3% terpenes, 0-5% sucralose, and 75-82% polysorbate 80. In another embodiment, said composition comprises 0.05-0.15% THC, 4.0-6.0% CBD, 0.5-2.5% ethyl pyruvate, 1.5-3.5% terpenes, 0-5% sucralose, and 82-92% polysorbate 80. In another embodiment, said composition comprises 0.4-0.6% THC, 5.5-7.5% CBD, 27-32% ethyl pyruvate, 1-3% terpenes, 0-5% sucralose, and 55-65% polysorbate 80.

In one embodiment, the composition comprises: 0-15% THC, 0-20% CBD, 0-50% ethyl pyruvate, 0-10% terpenes. In another embodiment, the composition comprises: 0-10% THC, 0-15% CBD, 0-50% ethyl pyruvate, 0-10% terpenes. In another embodiment, the composition comprises: 0-5% THC, 0-10% CBD, 0-40% ethyl pyruvate, 0-5% terpenes. In another embodiment, the composition comprises: 0.05-5% THC, 0.5-10% CBD, 1-40% ethyl pyruvate, 0-5% terpenes. In another embodiment, the composition comprises: 0.05-1% THC, 2.5-10% CBD, 1-40% ethyl pyruvate, 0-5% terpenes. In another embodiment, the composition comprises: 0.1-0.5% THC, 4-7.5% CBD, 1.0-10 or 10-30% ethyl pyruvate, 1-2.5% terpenes. In another embodiment, said composition comprises 50-90% polysorbate 80. In another embodiment, said composition comprises 0-5% sweetener, e.g., sucralose.

In another embodiment, said composition comprises: 0-7.0% THC, 0-7.0% CBD, 9-30% ethyl pyruvate, 1-5% terpenes, at least 59% polysorbate 80, and 0-5.0% sucralose.

In another embodiment, said composition comprises 0.25-1.0% THC, 4-9% CBD, 25-35% ethyl pyruvate, 1-3 terpenes, 0-5% sucralose, and 50-70% polysorbate 80. In another embodiment, said composition comprises 0.25-0.75% THC, 4-9% CBD, 7.5-12.5% ethyl pyruvate, 1-3% terpenes, 0-5% sucralose, and 70-85% polysorbate 80. In another embodiment, said composition comprises 0-0.25 THC, 3.5-9% CBD, 0.25-2.5% ethyl pyruvate, 1-3% terpenes, 0-5% sucralose, and 81.5-95.5% polysorbate 80. In another embodiment, said composition comprises 0.4-0.6% THC, 5.5-7.5% CBD, 7.5-12.5% ethyl pyruvate, 1-3% terpenes, 0-5% sucralose, and 75-82% polysorbate 80. In another embodiment, said composition comprises 0.05-0.15% THC, 4.0-6.0% CBD, 0.5-2.5% ethyl pyruvate, 1.5-3.5% terpenes, 0-5% sucralose, and 82-92% polysorbate 80. In another embodiment, said composition comprises 0.4-0.6% THC, 5.5-7.5% CBD, 27-32% ethyl pyruvate, 1-3% terpenes, 0-5% sucralose, and 55-65% polysorbate 80.

In another embodiment, the terpene in selected from any one, two, three, four, five, or all six of the terpenes selected from the group consisting of: myrcene, beta-caryophyllene, linalool, limonene, alpha-pinene, and eucalyptol. In a further embodiment, said terpenes include any one, two, three, four, or all five terpenes selected from the group consisting of: beta-caryophyllene, linalool, limonene, alpha-pinene, and eucalyptol. In a further embodiment, said formulation comprises 0-2.0% any one, two, three, four, or all five terpenes selected from the group consisting of: beta-caryophyllene, linalool, limonene, alpha-pinene, and eucalyptol. In a further embodiment, said formulation comprises 0.3-0.5% any one, two, three, four, or all five terpenes selected from the group consisting of: beta-caryophyllene, linalool, limonene, alpha-pinene, and eucalyptol. In a further embodiment, said formulation comprises 0.3-0.5% of each of the following terpenes: beta-caryophyllene, linalool, limonene, alpha-pinene, and eucalyptol. In a further embodiment, said formulation comprises: 0.25-0.45% beta-caryophyllene, 0-0.15% linalool, 0.5-1.0% limonene, 0.75-1.5% alpha-pinene, and 0-0.05% eucalyptol.

A composition of the present invention may further comprise, inter alia, an additional surfactant, antioxidant, viscosity modifying agent, cytochrome P450 metabolic inhibitor, P-GP efflux inhibitor, or semi-solid inducer. Preferred antioxidants include ascorbyl palmitate, butylated hydroxy anisole, butylated hydroxy toluene, propyl gallate, α-tocopherol, γ-tocopherol, and mixed tocopherols. In one embodiment, the composition of the present invention further comprises an antioxidant(s) in the range of about 0.01% w/v to about 0.1% w/v.

Viscosity modifying agents include unmodified starches, pregelatinized starches, crosslinked starches, guar gum, xanthan gum, acacia, tragacanth, carrageenans, alginates, chitosan, precipitated calcium carbonate (PCC), polyvinyl pyrrolidone, polyethylene oxide, polyethylene glycols (PEG), polycarbophils, EUDRAGIT® series polymers (E, L, S, RL, RS, NE), hydroxymethylpropyl cellulose (HPMC), hydroxyethylcellulose (HEC), hydroxypropylmethylcelluose (HPC), carboxymethylcellose sodium (Na-CMC), ethylcellulose, cellulose acetate, and cellulose acetate phthalate, polyvinylacetate/polyvinylpyrrolidone (PVA/PVP), PVA/PEG graft copolymer, hydrogenated vegetable oils, polyglycolized esters of fatty acids, carnauba wax, stearyl alcohol, and beeswax, polyvinyl caprolactam-polyvinyl acetate-polyethylene glycol graft co-polymer, and combinations thereof.

Cytochrome P450 inhibitors include an agent that inhibits pre-systemic hepatic first pass metabolism, e.g., d-α-tocopheryl polyethylene glycol 1000 succinate, anise oil, cinnamon oil, coriander oil, grapefruit oil, lemon oil, orange oil, peppermint oil, ascorbyl palmitate, propyl gallate, and combinations thereof.

PGP efflux inhibitors includes an agent that inhibits PGP induced cellular efflux mechanisms, e.g., polyethoxylated castor oil derivatives, polyoxyethylene sorbitan monooleate, polyoxyethylene glycerides, and combinations thereof.

A composition of the present invention may comprise a semi-solid inducer, e.g., colloidal silicon dioxide, granulated fumed silicas, precipitated silicas, amorphous silica gel, magnesium aluminum silicates, sodium magnesium aluminum silicates, microcrystalline cellulose, talc, dicalcium phosphate anhydrous, isomaltose and combinations thereof.

In addition to a primary surfactant(s), a composition of the present invention may further comprise an additional co-surfactant(s) to improve the emulsification of the provided compositions. Examples of co-surfactants include glycerol, sodium stearate, potassium laurate, sodium dodecyl sulfate, sodium sulfosuccinate, polyglycol, fatty acid esters, quaternary ammonium salts, amine hydrochlorides and combination thereof.

A composition may comprise chelating agents in a final range of about 0.01% to about 0.5% w/v. Examples of chelating agents include ethylenediaminetetraacetic acid (EDTA), phosphoric acid, polyphosphates, polysaccharides, citric acid and combinations thereof.

A composition may also additionally comprise inactive ingredients selected from a group consisting of antiadherents, binders, coatings, disintegrants, flavours, colours, lubricants, glidants, sorbents, preservatives, sweeteners, edible carriers, and combinations thereof.

A composition may further comprise a pH adjusting agent, e.g., disodium hydrogen phosphate, sodium acetate, sodium bicarbonate, sodium phosphate tribasic, dipotassium hydrogen phosphate, phosphoric acid, acetic acid, lactic acid, fumaric acid, adipic acid, malic acid, tartaric acid, citric acid, hydrochloric acid, sulfuric acid, salts thereof, and combinations thereof. In one embodiment, the composition pH is in the range of about 6.5 to about 7.5. In a further embodiment, the composition pH is in the range of about 7.0 to about 7.5. In a further embodiment, the composition pH is in the range of about 6.5 to about 7.0.

A composition may additionally comprise an osmotic agent, e.g., glycerin, glucose, sucrose, sorbitol, sodium phosphate and combinations thereof.

A composition may further comprise a sweetener, flavoring and/or taste-masking agent, e.g., glucose, fructose, sucrose, sorbitol, sucralose, saccharin sodium, aspartame, neotame, acesulfame potassium, stevioside, sodium chloride, D-limonene, citric acid, xylitol and combinations thereof. In one embodiment, the sweetener is selected from one or more of: acesulfame potassium, advantame, aspartame, neotame, saccharin, sucralose, stevia, glucose, fructose, sucrose, sorbitol, or xylitol. In one preferred embodiment, the sweetener is sucralose.

A composition may also further comprise preservatives, e.g., methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, and combinations thereof.

A composition of the present invention may be formulated, e.g., as a delayed release, sustained release, pulsatile release, immediate release, fast-disintegrating (e.g., orally disintegrating), or other release dosage form. The dosage form may include drug polymer conjugates, microencapsulation, controlled-release tablet/capsule coating, pH or other stimuli sensitive materials, enteric coated, or combinations thereof.

In another embodiment, the invention provides for an edible product comprising a composition of the present invention. Edible products include a lozenge, candy (including hard candies/boiled sweets, lollipop, gummy candy, candy bar, etc.), chocolates, brownie, cookie, trail bar, crackers, dissolving strip, mint, pastry, bread, etc. Further included is chewing gum, although the base gum is not consumed.

In another embodiment, a composition of present invention is a pharmaceutical composition. In another embodiment, the composition/pharmaceutical composition is a unit dose of the composition/pharmaceutical composition. In one embodiment, the unit dose is for oral administration, i.e., an oral unit dosage form. In another embodiment, the unit dose is for sublingual (held under the tongue) or buccal (held between the cheek and gum) administration, i.e., a sublingual or buccal unit dosage form. In a further embodiment, the unit dose is a liquid, solid, or semi-solid.

The unit dose (or serving) may be in the form of a syrup, drops, micellar dispersion, emulsion, solution, suspension, tablet, bolus, troche, tincture, oral/buccal/sublingual spray, lozenge, dissolving strip, or capsule. In one embodiment, the capsule is a hard gelatin capsule, a soft gelatin capsule, a starch capsule or an enteric coated capsule. In a one embodiment, the unit dose is a hard gelatin capsule. In a further embodiment, the unit dose is a soft gelatin capsule. In another embodiment, the syrup, drops, micellar dispersion, emulsion, solution, suspension, tablet, bolus, troche, tincture, spray, lozenge, or capsule is an oral unit dosage form and, in another embodiment, the same is a sublingual or buccal unit dosage form.

The specific dose of a composition of the present invention for any particular subject may depend upon a variety of factors including: the subject's age, body weight, general health, and gender; the time of administration; the combination of actives; the severity of the particular disease, disorder, or condition being treated; and the form or route of administration. Treatment dosages generally may be titrated to optimize safety and efficacy. A suitable dose for internal administration is generally in the range of 0.01 to 150 mg/kg per day, including 0.1-100 mg/kg, 0.1-50 mg/kg, and 0.1-10 mg/kg. Determining an appropriate dose can be performed by one of skill in the art.

The actives ingredients may be administered as a fixed dose combination or as separate doses. The actives ingredients may be administered as a single dosage form or as multiple dosage forms. The active ingredients may be administered concurrently or sequentially (either at a same or different time).

In one embodiment, the unit dose comprises about 0.25-100 mg of active ingredient, e.g., cannabinoid or cannabinoid extract. In another embodiment, the unit dose comprises about 0.25-0.5 mg of active ingredient, e.g., cannabinoid or cannabinoid extract. In another embodiment, the unit dose comprises about 0.5-1 mg of active ingredient, e.g., cannabinoid or cannabinoid extract. In another embodiment, the unit dose comprises about 1-2.5 mg of active ingredient, e.g., cannabinoid or cannabinoid extract. In another embodiment, the unit dose comprises about 2.5-5 mg of active ingredient, e.g., cannabinoid or cannabinoid extract. In another embodiment, the unit dose comprises about 5-7.5 mg of active ingredient, e.g., cannabinoid or cannabinoid extract.

In another embodiment, the unit dose comprises about 0.5-15 mg of active ingredient, e.g., cannabinoid(s) or cannabinoid extract. In another embodiment, the unit dose comprises about 0.5-2.5 mg of active ingredient, e.g., cannabinoid(s) or cannabinoid extract. In another embodiment, the unit dose comprises about 2.5-1 mg of active ingredient, e.g., cannabinoid(s) or cannabinoid extract. In another embodiment, the unit dose comprises about 2.5-5 mg of active ingredient, e.g., cannabinoid(s) or cannabinoid extract. In another embodiment, the unit dose comprises about 5-7.5 mg of active ingredient, e.g., cannabinoid(s) or cannabinoid extract. In another embodiment, the unit dose comprises about 5-10 mg of active ingredient, e.g., cannabinoid(s) or cannabinoid extract. In another embodiment, the unit dose comprises about 5-15 mg of active ingredient, e.g., cannabinoid(s) or cannabinoid extract. In another embodiment, the unit dose comprises about 7.5-10 mg of active ingredient, e.g., cannabinoid(s) or cannabinoid extract. In another embodiment, the unit dose comprises about 10-12.5 mg of active ingredient, e.g., cannabinoid(s) or cannabinoid extract. In another embodiment, the unit dose comprises about 12.5-15 mg of active ingredient, e.g., cannabinoid(s) or cannabinoid extract. In another embodiment, the unit dose comprises about 15-20 mg of active ingredient, e.g., cannabinoid(s) or cannabinoid extract. In another embodiment, the unit dose comprises about 20-30 mg of active ingredient, e.g., cannabinoid(s) or cannabinoid extract. In another embodiment, the unit dose comprises about 30-40 mg of active ingredient, e.g., cannabinoid(s) or cannabinoid extract. In another embodiment, the unit dose comprises about 40-50 mg of active ingredient, e.g., cannabinoid(s) or cannabinoid extract. In another embodiment, the unit dose comprises about 50-60 mg of active ingredient, e.g., cannabinoid(s) or cannabinoid extract. In another embodiment, the unit dose comprises about 60-70 mg of active ingredient, e.g., cannabinoid(s) or cannabinoid extract. In another embodiment, the unit dose comprises about 70-75 mg of active ingredient, e.g., cannabinoid(s) or cannabinoid extract. In another embodiment, the unit dose comprises about 70-80 mg of active ingredient, e.g., cannabinoid(s) or cannabinoid extract. In another embodiment, the unit dose comprises about 80-90 mg of active ingredient, e.g., cannabinoid(s) or cannabinoid extract. In another embodiment, the unit dose comprises about 90-100 mg of active ingredient, e.g., cannabinoid(s) or cannabinoid extract. In another embodiment, the unit dose comprises about 100-150 mg of active ingredient, e.g., cannabinoid(s) or cannabinoid extract. In another embodiment, the unit dose comprises about 150-200 mg of active ingredient, e.g., cannabinoid(s) or cannabinoid extract. In another embodiment, the unit dose comprises about 0.5, about 1, about 5, about 7.5, about 10, about 12.5 mg or about 15 mg of active ingredient, e.g., cannabinoid(s) or cannabinoid extract. In some embodiments, the cannabinoid is THC. In some embodiments, the cannabinoid is CDB. In other embodiments, the cannabinoids are THC and CBD.

In one embodiment, the unit dose comprises an amount of one or more cannabinoids, wherein the amount of each cannabinoid, independently, or total cannabinoids (if specified) is selected from the group consisting of: none, trace amount, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, and >200 mg. In a further embodiment, said one or more cannabinoids is CBD, THC, CBN, CBG, CBC, THCA, CBDA, and THCV, and said unit dose comprises an amount of said CBD, THC, CBN, CBG, CBC, THCA, CBDA, and THCV, each independently selected from the group consisting of: none, trace amount, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, and >200 mg. In a further embodiment, said one or more cannabinoids is CBD and THC, and said unit dose comprises an amount of said CBD and THC independently selected from the group consisting of: none, trace amount, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, and >200 mg. In a further embodiment, said one or more cannabinoids is delta-8-THC and delta-9-THC, and said unit dose comprises an amount of said delta-8-THC and delta-9-THC independently selected from the group consisting of: none, trace amount, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, and >100 mg.

In one embodiment, the unit dose comprises an amount of one or more terpenes, independently, or total terpenes selected from the group consisting of: none, trace amount, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, and >200 mg. In a further embodiment, said one or more terpenes is selected from: myrcene, beta-caryophyllene, linalool, limonene, alpha-pinene, and eucalyptol, and said unit dose comprises an amount of said myrcene, beta-caryophyllene, linalool, limonene, alpha-pinene, and eucalyptol, each independently selected from the group consisting of: none, trace amount, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, and >200 mg.

In one embodiment, said comprises the step of administering a therapeutically effective amount of at least two active ingredients selected from: one or more cannabinoids, and/or ethyl pyruvate, and/or one or more terpenes (including physiologically/pharmaceutically acceptable salts, solvates, hydrates, and co-crystals thereof). In another embodiment, said active ingredients comprise one or more cannabinoids and ethyl pyruvate. In another embodiment, said active ingredients comprise ethyl pyruvate and one or more terpenes. In another embodiment, said active ingredients comprise one or more cannabinoids and one or more terpenes. In another embodiment, said active ingredients comprise one or more cannabinoids, ethyl pyruvate, and one or more terpenes. In another embodiment, said active ingredients comprise at least two cannabinoids. In another embodiment, said active ingredients comprise at least two terpenes. In another embodiment, said active ingredients comprise one or more cannabinoids and at least one other active ingredient; ethyl pyruvate and at least one other active ingredient; or one or more terpenes and at least one other active ingredient.

In one embodiment, the one or more cannabinoids is selected from the group consisting of: CBD, THC, CBN, CBG, CBC, THCA, CBDA, and THCV. In another embodiment, the one or more cannabinoids is CBD, THC, or CBD and THC. In one embodiment, the active ingredients comprise: one or more cannabinoids selected from CBD, THC, or CBD and THC; and ethyl pyruvate.

In another embodiment, the active ingredients comprise one or more terpenes. In another embodiment, the terpene in selected from any one, two, three, four, five, or all six of the terpenes selected from the group consisting of: myrcene, beta-caryophyllene, linalool, limonene, alpha-pinene, and eucalyptol. In a further embodiment, the one or more terpenes is any one, two, three, four, or all five selected from beta-caryophyllene, linalool, limonene, alpha-pinene, or eucalyptol. In a further embodiment, the active ingredients comprise: one or more cannabinoids selected from CBD, THC, or CBD and THC; ethyl pyruvate; and one or more terpenes selected from any one, two, three, four, five, or all six of: myrcene, beta-caryophyllene, linalool, limonene, alpha-pinene, or eucalyptol.

In one embodiment, the unit dose comprises an amount of ethyl pyruvate selected from the group consisting of: none, trace amount, 0.1-0.5 mg, 0.5-1.0 mg, 1.0-2.5 mg, 2.5-5 mg, 1.0-25 mg, 25-50 mg, 50-75 mg, 75-100 mg, 5.0-75 mg, 5.0-10 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, and 1-100 mg 50-100 mg, 100-200 mg, 200-300 mg, 300-400 mg, 500-600 mg, 600-700 mg, 700-800 mg, 800-900 mg, 900-1000 mg; 1.0-2.0 g, 2.0-3.0 g, 3.0-4.0 g, 4.0-5.0 g, 5.0-6.0 g, 6.0-7.0 g, 7.0-8.0 g, 8.0-9.0 g, 9.0-10 g, 10-11 g, 11-12 g, 12-12.5, or >12.5 g.

In one embodiment, the unit dose comprises:

THC/THC extract: 0-10 mg;

CBD/CBD extract: 0-50 mg;

ethyl pyruvate: 50-250 mg; and

terpenes: 0-20 mg total or 0-3.5 mg/terpene.

In one embodiment, the unit dose comprises:

a) an amount of one or more cannabinoids, the amount of each cannabinoid independently selected from the group consisting of: none, trace amount, 0.01-0.05 mg, 0.05-0.1 mg, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, and >200 mg;

b) an amount of ethyl pyruvate selected from the group consisting of: none, trace amount, 0.1-0.5 mg, 0.5-1.0 mg, 1.0-2.5 mg, 2.5-5 mg, 1.0-25 mg, 25-50 mg, 50-75 mg, 75-100 mg, 5.0-75 mg, 5.0-10 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, and 1-100 mg 50-100 mg, 100-200 mg, 200-300 mg, 300-400 mg, 500-600 mg, 600-700 mg, 700-800 mg, 800-900 mg, 900-1000 mg; 1.0-2.0 g, 2.0-3.0 g, 3.0-4.0 g, 4.0-5.0 g, 5.0-6.0 g, 6.0-7.0 g, 7.0-8.0 g, 8.0-9.0 g, 9.0-10 g, 10-11 g, 11-12 g, 12-12.5, or >12.5; and

d) an amount of one or more terpenes, each independently, or total terpenes selected from the group consisting of: none, trace amount, none, trace amount, 0.01-0.05 mg, 0.05-0.1 mg, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, and >200 mg.

In a further embodiment, said one or more cannabinoids is CBD, THC, CBN, CBG, CBC, THCA, CBDA, and THCV, and said unit dose comprises an amount of said CBD, THC, CBN, CBG, CBC, THCA, CBDA, and THCV, each independently selected from the group consisting of: none, trace amount, none, trace amount, 0.01-0.05 mg, 0.05-0.1 mg, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, and >200 mg.

In a further embodiment, said one or more cannabinoids is CBD and THC, and said unit dose comprises an amount of said CBD and THC each independently selected from the group consisting of: none, trace amount, none, trace amount, 0.01-0.05 mg, 0.05-0.1 mg, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, and >200 mg.

In a further embodiment, said one or more cannabinoids is delta-8-THC and delta-9-THC, and said unit dose comprises an amount of said delta-8-THC and delta-9-THC each independently selected from the group consisting of: none, trace amount, none, trace amount, 0.01-0.05 mg, 0.05-0.1 mg, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, and >200 mg.

In a further embodiment, said one or more terpenes is beta-caryophyllene, linalool, limonene, alpha-pinene, and eucalyptol, and said unit dose comprises an amount of said beta-caryophyllene, linalool, limonene, alpha-pinene, and eucalyptol, each independently selected from the group consisting of: none, trace amount, none, trace amount, 0.01-0.05 mg, 0.05-0.1 mg, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, and >200 mg.

In another embodiment, said unit dose further comprises one or more additional active ingredients selected from: one or more non-opioid analgesic/anti-inflammatory drug, one or more anti-migraine drug, one or more anti-emetic, one or more anti-Parkinson disease drug, one or more anti-MS disease drug, one or more anti-spasticity drug, one or more nutraceutical, one or more corticosteroid, or a combination thereof. In a further embodiment, said unit dose comprises an amount of each of said one or more additional active ingredients that is independently selected from the group consisting of: 0.001-0.01 mg, 0.01-0.05 mg, 0.05-0.1 mg, 0.1-0.5 mg, 0.25-1 mg, 0.5-15 mg, 0.5-2.5 mg, 1.0-2.5 mg, 2.5-5 mg, 5.0-7.5 mg, 5.0-10 mg, 1.0-25 mg, 25-50 mg, 50-75 mg, 75-100 mg, 10-20 mg, 10-15 mg, and 15-20 mg, 20-30 mg, 30-40 mg, 40-50 mg, 50-60 mg, 60-70 mg, 70-80 mg, 80-90 mg, 90-100 mg, 1-100 mg, 100-125 mg, 125-150 mg, 150-175 mg, 175-200 mg, 200-300 mg, 300-400 mg, 500-600 mg, 600-700 mg, 700-800 mg, 800-900 mg, 900-1000 mg; 1.0-2.0 g, 2.0-3.0 g, 3.0-4.0 g, 4.0-5.0 g, 5.0-6.0 g, 6.0-7.0 g, 7.0-8.0 g, 8.0-9.0 g, 9.0-10 g, 10-11 g, 11-12 g, 12-12.5, or >12.5.

In one embodiment, the unit dose comprises about 25-7,500 mg of active ingredients. In some embodiments, the unit dose comprises about 25-50 mg, 50-100 mg, 100-600 mg, 200-700 mg, 300-800 mg, 400-900 mg, 500-1000 mg, 1.0-2.0 g, 2.0-3.0 g, 3.0-4.0 g, 4.0-5.0 g, 5.0-6.0 g, 6.0-7.0 g, 7.0-8.0 g, 8.0-9.0 g, 9.0-10 g, 10-11 g, 11-12 g, 12-12.5, or >12.5 of active ingredients.

A second aspect provides a method of making a composition of the present invention, said method comprising the steps of:

providing at least one active ingredient and at least one surfactant; and

combining said at least one active ingredient and said at least one surfactant to form a mixture. In one embodiment, the mixture is an isotropic or homogeneous mixture.

In one embodiment, at least one active ingredient is selected from a cannabinoid, cannabinoid extract, terpene, or terpene extract. In a further embodiment, at least one active ingredient is a cannabinoid or cannabinoid extract.

In some embodiments, the invention provides a method of making a composition of the present invention, said method comprising the steps of:

providing at least one active ingredient; at least one surfactant; and, optionally, one or more fatty acid, monoglyceride, diglyceride, triglyceride, or a combination thereof;

combining said at least one active ingredient; said at least one surfactant; and, optionally, one or more fatty acid, monoglyceride, diglyceride, triglyceride, or a combination thereof, to form a mixture. In one embodiment, the mixture is an isotropic or homogeneous mixture.

In one embodiment, at least one active ingredient is selected from a cannabinoid, cannabinoid extract, terpene, or terpene extract. In a further embodiment, at least one active ingredient is a cannabinoid or cannabinoid extract.

In one embodiment, the method of making the composition of the first aspect comprises the steps of:

providing an active ingredient, at least one surfactant, and at least one triglyceride; and

combining said active ingredient, said surfactant(s), and said triglyceride to form a mixture. In one embodiment, the mixture is an isotropic or homogeneous mixture. In some embodiments, the triglyceride is an MCT or LCT, as provided herein. In one embodiment the active ingredient is selected from a cannabinoid, cannabinoid extract, terpene, or terpene extract.

In a further embodiment, the active ingredient is a cannabinoid or cannabinoid extract.

In one embodiment, said at least one active ingredient is one or more cannabinoids, and/or ethyl pyruvate, and/or one or more terpenes.

In another embodiment, the method of making the composition of the first aspect comprises the steps of:

Providing at least one active ingredient; at least one surfactant; and at least one triglyceride; wherein said at least one surfactant comprises one or more selected from polysorbate 80, polyethylene glycol (PEG) 40 hydrogenated castor oil (Kolliphor RH40), D-α-Tocopherol polyethylene glycol 1000 succinate (TPGS), lauroyl macrogol 32 glycerides (e.g., GELUCIRE® 44/14), or a combination thereof; and, wherein said triglyceride is a medium-chain triglyceride and/or long-chain triglyceride; and

combining said at least one active ingredient; said at least one surfactant(s); and said triglyceride to form a mixture. In one embodiment, the mixture is an isotropic or homogeneous mixture. In some embodiments, the triglyceride is an MCT or LCT, as provided herein.

In one embodiment, at least one active ingredient is selected from a cannabinoid, cannabinoid extract, terpene, or terpene extract. In a further embodiment, at least one active ingredient is a cannabinoid or cannabinoid extract.

The invention further provides for a method for increasing at least one parameter selected from the group consisting of solubility, dissolution, oral bioavailability, Cmax, absorption, onset of action, for decreasing time to Tmax, or for decreasing intra-patient variability comprising the steps of:

providing at least one active ingredient; at least one surfactant; and, optionally, one or more fatty acid, monoglyceride, diglyceride, triglyceride, or a combination thereof;

combining said active ingredient; said surfactant; and, optionally, a fatty acid, monoglyceride, diglyceride, triglyceride, or a combination thereof to form an isotropic or homogeneous mixture. In some embodiments, the triglyceride is an MCT or LCT, as provided herein.

In one embodiment, at least one active ingredient is selected from a cannabinoid, cannabinoid extract, terpene, or terpene extract. In a further embodiment, at least one active ingredient is a cannabinoid or cannabinoid extract. In one embodiment, said at least one active ingredient is one or more cannabinoids, and/or ethyl pyruvate, and/or one or more terpenes.

The formulations of the present invention can significantly decrease the amount of time for the onset of action of the active ingredient. In one embodiment, the composition, e.g., cannabinoid composition, of the present invention has an onset of action within 15 minutes, 15-20 minutes, 20 minutes, 25 minutes, 30 minutes, or within 45 minutes post administration.

The formulations of the present invention can further significantly decrease the peak time (the time it takes for an active ingredient to reach maximum effect) of the active ingredient. In one embodiment, the composition, e.g., cannabinoid composition, of the present invention has a peak time within 90 minutes, within 80 minutes, within 70 minutes, within 60-70 minutes, within 60 minutes, within 50 minutes, within 45-60 minutes, within 45 minutes, within 40 minutes, or within 30 minutes post administration.

The formulations of the present invention can further significantly increase the peak effect, i.e., the maximum effect of the active ingredient, e.g., the psychotropic effect of THC.

In one embodiment, the method for enhancing at least one parameter selected from the group consisting of solubility, dissolution, oral bioavailability and absorption comprises the steps of:

providing at least one active ingredient, at least one surfactant, and at least one triglyceride, and combining said active ingredient(s), said surfactant(s) and said triglyceride(s) to form a mixture. In one embodiment, the mixture is an isotropic or homogeneous mixture. In some embodiments, the triglyceride is an MCT or LCT, as provided herein.

In one embodiment, the active ingredient is selected from a cannabinoid, cannabinoid extract, terpene, or terpene extract. In a further embodiment, the active ingredient is a cannabinoid or cannabinoid extract.

In another embodiment, said at least one triglyceride comprises a medium-chain triglyceride and/or long-chain triglyceride, and said at least one surfactant comprises one or more selected from polysorbate 80, polyethylene glycol (PEG) 40 hydrogenated castor oil (Kolliphor RH40), or D-α-Tocopherol polyethylene glycol 1000 succinate (TPGS) and/or lauroyl macrogol 32 glycerides. In one embodiment, the mixture is an isotropic or homogeneous mixture.

A third aspect of the present invention provides for a method of treating a disease or disorder in a subject (e.g., human) who would benefit from at least one active ingredient of the present invention, the method comprising the step of administering an effective amount of a composition of the present invention to said subject. Preferably, the subject is a human. In a preferred embodiment, said composition comprises at least one cannabinoid, cannabinoid extract, terpene, terpene extract, or a combination thereof.

In one embodiment, the disease or disorder is selected from: Alzheimer Disease, Amyotrophic Lateral Sclerosis (ALS), Multiple Sclerosis (MS), spasticity, pain, anxiety, nausea, vomiting, insomnia, restless leg syndrome (RLS), diabetes mellitus, dystonia, epilepsy, fibromyalgia, gastrointestinal disorders, inflammation or inflammatory condition, inflammatory bowel disease, Crohn's disease, irritable bowel syndrome, gliomas, cancer, Hepatitis C, Human Immunodeficiency Virus (HIV) Huntington Disease, hypertension, incontinence, methicillin-resistant Staphyloccus aureus (MRSA), multiple sclerosis, osteoporosis, pruritus, rheumatoid arthritis, insomnia, sleep apnea, or Tourette Syndrome.

In one embodiment, the disease, condition, or pathology is inflammation or an inflammatory condition.

In a further embodiment, the method includes reducing the level of a cytokine in a mammal, wherein said cytokine is selected from the group consisting of: tumor necrosis factor (TNF), interferon-gamma (IFN-gamma), interleukin-1-beta (IL-1-beta), and high mobility group B-1 (HMGB-1).

In a further embodiment, the method includes inhibiting IL-1β-induced inflammatory and catabolic pathways, preferably, NF-κB and JNK activation, and the expression of inflammatory (iNOS) and catabolic (MMP-1 and -13) genes. In a further embodiment, the method includes reducing MCP-1 production via NF-kB activation. In a further embodiment, the method includes reducing NO production, reducing IFN-gamma, reducing IL-4, IL-6, or for increasing IL-10.

In one embodiment, the inflammation or an inflammatory condition is selected from the group consisting of: asthma, cachexia secondary to acquired immune deficiency syndrome (AIDS), cachexia secondary to infection or malignancy, chronic obstructive pulmonary disease (COPD), Crohn's disease, endotoxic shock, fever and myalgia due to infection, gouty arthritis and other arthritic conditions, graft v. host rejection, gram negative sepsis, keloid formation, multiple sclerosis, osteoarthritis, psoriasis and eczema, pulmonary fibrosis, pulmonary sarcoidosis, reperfusion injury, rheumatoid arthritis, rheumatoid spondylitis, scar tissue formation, sepsis, septic shock, silicosis, toxic shock syndrome, and ulcerative colitis.

In one embodiment, the disease, condition or pathology in a mammal (e.g., human) is a central nervous system (CNS) disorder. In a further embodiment, the CNS disorder is selected from any one of: Alzheimer's disease, amyotrophic lateral sclerosis, adrenoleukodystrophy, brain injury, cerebral infarction, corticobasal degeneration (CBD), Creutzfeldt-Jakob Disease, epilepsy, Friedrich's ataxia, frontal lobe degeneration (frontotemporal dementia), geriatric dementia, Huntington's disease, ischemic stroke, Lewy body dementia, multi-infarct dementia, multiple sclerosis, multiple system atrophy (MSA), olivopontocerebe/laratrophy (OPCA), Parkinsonian disorders, Parkinson's disease, Pick's Disease, progressive supranuclear palsy, Shy-Drager syndrome, spinal cord injury, spasticity, spinal ischemia, striatonigral degeneration (SND), stroke, vascular dementia. In a further embodiment, the spasticity due to any one of the following: multiple sclerosis, spinal cord injury, stroke, brain injury, or amyotrophic lateral sclerosis. In a preferred embodiment, the spasticity is due to multiple sclerosis. In another embodiment, the CNS disorder is hyperreflexia. In a further embodiment, the hyperreflexia due to any one of the following: multiple sclerosis, spinal cord injury, stroke, brain injury, or amyotrophic lateral sclerosis. In a preferred embodiment, the hyperreflexia is due to multiple sclerosis.

In one embodiment, the CNS disorder is a demyelinating condition. In a further embodiment, the demyelinating condition is selected from any one of the following: acute disseminated encephalomyelitis, acute transverse myelitis, acute viral encephalitis, adrenoleukodystrophy (ALD), adrenomyeloneuropathy (AMN), AIDS-vacuolar myelopathy, Binswanger's disease (subcortical leukoencephalopathy), central pontine myelinolysis (CPM), disseminated necrotizing leukoencephalopathy (DNL), HTLV-associated myelopathy, Leber's hereditary optic atrophy, leukodystrophy, multiple sclerosis (MS), multiple sclerosis variants such as Neuromyelitis Optica (Decic's Disease), Diffuse Sclerosis, Transitional Sclerosis, Acute Disseminated Encephalomyelitis, and Optic Neuritis, progressive multifocal leukoencephalopathy (PML), radiation necrosis, Schilder's disease, subacute sclerosing panencephalitis, or tropical spastic paraparesis. In a preferred embodiment, the demyelinating condition is multiple sclerosis. In a further embodiment, the method is for slowing the progression of MS. In one embodiment, a human with MS is treated over at least about a time selected from the group consisting of: 4 weeks, 8 weeks, 12 weeks, 16 weeks, 20-25 weeks, 25-35 weeks, 35-45 weeks, 45-52 weeks, and more than 52 weeks. In a further embodiment, the human with MS (MS patient) that is treated with the active ingredients or composition of the invention displays an improvement in an outcome measure selected from the group consisting of: Ashworth score, Rivermead Mobility Index, 6-minute walk test, timed 25 feet walk test, timed 10-meter walk test, Dynamic Gait Index, Timed Up and Go test, EDSS (extended disability status scale), and appearance of exacerbations or MRI (magnetic resonance imaging); wherein said MS patient treated with the active ingredients or composition of the present invention is compared to a MS patient administered placebo over the same time. The comparison is preferably between a treated cohort and placebo cohort wherein a measure of therapeutic effectiveness in this regard is a statistically significant difference. The patient or cohorts may be treated with using the best standard of care which may include treatment with one or more MS drugs. In any case, the slowing of progression of MS is due to the composition of the invention, and not the results of natural periods of remission between attacks. In a further embodiment, the MS is relapsing remitting MS. In another embodiment, the MS is primary progressive MS.

The EDSS is a means to grade clinical impairment due to MS (Kurtzke, Neurology. 1983 November; 33(11):1444-52). The scale ranges from 0 (normal) to 10 (death due to MS). In one embodiment, a decrease of one full step (one point) defines an effective treatment in the context of the present invention (Kurtzke, Ann Neurol. 1994; 36 Suppl:573-9). In one embodiment, the invention provides for a method of reducing an MS patient's EDSS scale by at least one full step comprising the step of treating said MS patient with a therapeutically effective amount of the active ingredients or composition of the present invention over at least about a time selected from the group consisting of: 4 weeks, 8 weeks, 12 weeks, 16 weeks, 20-25 weeks, 25-35 weeks, 35-45 weeks, 45-52 weeks, and more than 52 weeks.

Exacerbations are defined as the appearance of a new symptom that is attributable to MS and accompanied by an appropriate new neurologic abnormality. The exacerbation must last at least 24 hours and be preceded by stability or improvement for at least 30 days. Exacerbations are mild, moderate, or severe according to changes in a Neurological Rating Scale (Sipe et al., Neurology. 1984 October; 34(10):1368-72.). An annual exacerbation rate and proportion of exacerbation-free patients are determined. In one embodiment, therapy is deemed to be effective if there is a statistically significant difference in the rate or proportion of exacerbation-free patients between the treated group and the placebo group for either of these measurements. A measure of effectiveness as therapy in this regard is a statistically significant difference in the time to first exacerbation or duration and severity in the treated group compared to control group.

Mill can be used to measure active lesions. The presence, location, and extent of MS lesions are determined by radiologists. Three analyses may be done: evidence of new lesions, rate of appearance of active lesions, percentage change in lesion area (Paty et al., Neurology 43:665, 1993). In one embodiment, improvement due to therapy is established when there is a statistically significant improvement in an individual patient compared to baseline or in a treated group versus a placebo group.

In one embodiment, therapy is effective when there is an improvement in one or more disabling neurological impairments such as blindness, paralysis, incoordination, and bowel or bladder dysfunction, as well as a less apparent symptom such as fatigue. As used herein “fatigue” includes loss of power, capacity to respond to stimulation, or the tiredness, or sleepiness associated with multiple sclerosis. In another embodiment, therapy is effective where there is a lessoning of a disorder caused by an impairment in the function of one or more of the following systems: pyramidal, cerebella, brainstem, sensory, bowel and bladder, visual, cerebral or other neurologic abnormality. In a further embodiment, the pyramidal function is selected from the development of: paraparesis, hemiparesis, monoparesis, quadriparesis, monoplegia, paraplegia, quadriplegia, or hemiplegia. In another embodiment, the system is cerebella and the disorder is selected from the development of ataxia, including truncal or limb ataxia. In another embodiment, the system is brainstem and the disorder is selected from the development of nystagmus, extraocular weakness, or dysarthria. In another embodiment, treatment is effective when there is a reduction in: the inflammatory response in the brain and other regions of the nervous system, breakdown or disruption of the blood-brain barrier, appearance of lesions in the brain, tissue destruction, demyelination, autoimmune inflammatory response, acute or chronic inflammatory response, neuronal death, and/or neuroglia death.

In one embodiment, the pain is chronic pain. In another embodiment, the pain is acute pain. In a further embodiment, the acute pain is a migraine. In a further embodiment, the pain is selected from any one or more of the following: post-herpetic neuralgia, trigeminal neuralgia, spinal cord injury pain, carpal tunnel syndrome, phantom limb, ischemic pain, pain resulting from sports injuries, back pain (e.g., low back pain), menstrual pain, gastrointestinal or urethral cramps, skin wounds, burns, or cancer pain. In a preferred embodiment, the pain is cancer pain.

In another embodiment, the nausea and/or vomiting results from a chemotherapy, e.g., cancer chemotherapy. In another embodiment, the nausea and/or vomiting results from opioid use.

In another embodiment, the method is for increasing socialization, increasing relaxation, inducing sleep, reducing the time needed to fall asleep, or for inducing a psychotropic effect (commonly known as a “high”). In another embodiment, the method is for reducing the amount of opioid(s) used by a subject suffering from pain or used by a subject addicted to an opioid.

The composition may be administered once, twice, three, or four times a day, or as needed.

In one embodiment, the invention provides a method of reducing the intensity or duration of pain in a subject (i.e., a subject, e.g., human), in need thereof, comprising the step of administering to the subject an effective amount of a cannabinoid containing composition of the present invention. In a further embodiment, the method decreases pain intensity in the subject. In a further embodiment, the method decreases pain duration in the subject. In one embodiment, the pain is acute pain. In another embodiment, the pain is chronic pain. In some embodiments, the subject has reduced pain intensity for at least 4 hours, at least 6 hours, at least 8 hours, at least 12 hours, at least 18 hours, or at least 24 hours post administration. In one embodiment, the cannabinoid composition of the present invention has a maximum pain relieving effect between 1-4 hours or between 1.5-2.5 hours post administration. In another embodiment, the cannabinoid composition of the present invention has an onset of pain relieving effect within 15 minutes, 20 minutes, 25 minutes, 30 minutes, or within 45 minutes post administration.

In one embodiment, the invention provides a method of reducing or preventing nausea or vomiting in a subject in need thereof, comprising administering to the subject an effective amount of a cannabinoid containing composition of the present invention. In one embodiment, the nausea or vomiting is opioid induced nausea or vomiting. The opioid inducing the nausea or vomiting may be an opioid analgesic such as hydrocodone, oxycodone, oripavine, dihydromorphine, hydromorphinol, nicomorphine, dipropanoylmorphine, diacetyldihydromorphine, desomorphine, methyldesorphine, heterocodeine, benzylmorphine, dihydroheterocodeine, myrophine, pentamorphone, tramadol, fentanyl, etc. In one embodiment, the cannabinoid containing composition is administered 0-30 minutes, 30-60 minutes prior to administration of the opioid. In another embodiment, the cannabinoid containing composition is administered 60 minutes prior to administration of the opioid. In another embodiment, the cannabinoid containing composition is administered concurrently with the administration of the opioid. In one embodiment, the nausea or vomiting occurs after surgery and results from anesthesia.

In one embodiment, the subject has reduced intensity of nausea in the 2, hours, 3 hours, 4 hours, 5 hours, 6 hours, 8 hours, 12 hours, 18 hours, or 24 hours following initial administration of the cannabinoid containing composition. In one embodiment, the subject has reduced vomiting in the 4 hours, 6 hours, 8 hours, 12 hours, 18 hours, or 24 hours following initial administration of the cannabinoid containing composition. In one embodiment, the cannabinoid composition of the present invention has a maximum nausea or vomiting reducing effect between 1-4 hours, 1-3 hours, 2-4 hours, or between 1.5-2.5 hours post administration. In another embodiment, the cannabinoid composition of the present invention has an onset of nausea or vomiting reducing effect within 15 minutes, 20 minutes, 25 minutes, 30 minutes, or within 45 minutes post administration.

In one embodiment, the method of reducing nausea or vomiting in a subject includes reducing the occurrence of nausea or vomiting.

In one embodiment, the composition of the present invention has a Tmax that is about 1-6 hours. In a further embodiment, the Tmax is about 1-3 hours in a fasted subject. In a further embodiment, the Tmax is about 2-4 hours in a fasted subject.

In another embodiment, the composition of the present invention has an about 20-400% greater absorption in the 90 minutes following administration than MARINOL®. In another embodiment, the composition of the present invention has an about 20-400% greater absorption, 100-200%, 200-300%, or 300-400% in the 60 minutes following administration than MARINOL®.

In another embodiment, the composition of the present invention has an about 20-400%, 100-200%, 200-300%, or 300-400% less first-pass metabolism than MARINOL®.

### EXAMPLES

Cannabidiol was procured from CBD internationals and marijuana THC extract was procured from New England Treatment Access (NETA). GELUCIRE® 44/14, Peceol, Transcutol, Lauroglycol 90, Capryol 90, Labrafac 1349 and Geloil samples were from Gattafosse SAS, Saint-Priest, France. Poloxamer 124, PEG 25, PEG 400 and polyoxyethylene 10 oleyl ether (Oleth-10 or BRIJ 97) were procured from VWR. Vitamin E TPGS (d-alpha tocopheryl polyethylene glycol 1000 succinate) was procured from Antares health products. Polysorbate 80 was procured from Modernist Pantry and Solutol® HS 15 (Kolliphor® HS 15) was procured from BASF. Solutol® HS 15 is a tradename for macrogol 15 hydroxystearate (also called polyoxyl 15 hydroxystearate) and contains soluble non-ionic surfactants (70%) and PEG (3) formed by the reaction of 12-hydroxystearic acid with ethylene oxide at alkaline pH (12).

GELUCIRE® 44/14 (Gattefossé) is a tradename for lauroyl macrogol 32 glycerides (synonyms: lauroyl polyoxyl-32 glycerides, PEG-32 lauroyl polyoxylglycerides or PEG-32 lauric glycerides) that is obtained by polyglycolysis of hydrogenated coconut oil (medium and long chain triacylglycerols) and PEG-32. It is composed of a defined admixture of C8-C18 mono-, di- and triacylglycerols (20% w/w), PEG-32 mono- and diesters and free PEG-32 (80% w/w). The main fatty acid present is lauric acid which accounts for 45% on average of the total fatty acids. See Jannin, V. OCL 16(4):267-272 (2009).

Compositions comprising of long chain triglycerides or medium chain triglycerides with a variety of surfactants were prepared and tested to determine whether they produce micro- and nano-emulsions via self-emulsifying mechanisms. Formation of self-emulsification was assessed using visual and particle size analysis.

Single Excipient Dissolution Studies:

1 g Cannabidiol (CBD) or THC extract was added to a 20 mL scintillation vial to which was added 10 mL of excipient (9 g) (surfactant or triglyceride). The resulting solution was stirred for 30 minutes at 25° C. in case of liquid excipients. Semisolid and solid excipients were heated to 80° C. (to convert them into a liquid state) and stirred for 30 minutes. Stirring was continued until CBD or THC was completely soluble in the excipient forming a clear solution. This clear solution was used for dissolution studies in water by adding 45 microliter in 12 mL water (0.375%) with continuous stirring at 25° C. The resulting emulsion was stirred for 2 hours before particle size measurement. The particle size was measured using Dynamic Light Scattering instrument (Malvern Zetasizer Nano).

In single excipient studies, all oils and surfactants demonstrated high solubility. To determine whether these excipients are self-emulsifying with cannabinoids, dilution studies in water were performed. The data for both CBD and Cannabinoid extract (Table 3) showed that oils do not form microemulsions, which was expected.

TABLE 3

| | | | | | Particle size |
|---|---|---|---|---|---|
| | | HLB | | Particle size | (Cannabinoid |
| Excipient | Type | Value | Emulsion | (CBD) | extract) |
| Poloxamer 124 | Surfactant | 16 | nanoemulsion | 39 nm | 66 nm |
| GELUCIRE ® 44/14 | Surfactant | 11 | nanoemulsion | 44 nm | 27 nm |
| TPGS | Surfactant | 13 | nanoemulsion | 47 nm | 50 nm |
| SOLUTOL ® HS 15 | Surfactant | 15 | nanoemulsion | 18 nm | 17 nm |
| PEG 25 | Co-solvent | 11 | nanoemulsion | 96 nm | 165 nm |
| Polysorbate 80 | Surfactant | 15 | nanoemulsion | 65 nm | 89 nm |
| PEG 400 | Co-solvent | 10 | microemulsion | 382 nm | 321 nm |
| BRIJ 97 | Surfactant | 12 | microemulsion (CBD); | 212 nm | 35 nm |
| | | | nanoemulsion (THC) | | |
| Peceol | Oil (LCT) | 2 | Phase separation | — | |
| Transcutol | Surfactant | 4 | Phase separation | — | |
| Lauroglycol 90 | Surfactant | 3 | Phase separation | — | |
| Capryol 90 | Oil (MCT) | 6 | Phase separation | — | |
| Labrafac 1349 | Oil (MCT) | 1 | Phase separation | — | |
| Geloil | Oil/Surfactant | 5 | Phase separation | — | |

The results showed that some surfactants and co-solvents form micro- or nano-emulsions while others do not. Successful surfactants and surfactant/co-solvent combinations were empirically selected based on experimental observation. The results confirm that empirical studies are necessary to identity compositions that efficiently self-emulsify to form stable micro- or nano-emulsions.

The single excipient data was used as an initial screen for candidate surfactants. The candidate surfactants were then used in compositions (both binary and ternary) that were screened to determine whether they were self-emulsifying.

Binary and Ternary Formulation Dissolution Studies:

THC extract, TPGS, GELUCIRE® 44/14, Polysorbate 80 (PS 80), LCT oil and MCT oil were mixed in a ratio as shown in Table 4 in a 20 mL scintillation vials.

TABLE 4

| | Extract | MCT | LCT | TPGS | GELUCIRE | PS 80 | Particle |
|---|---|---|---|---|---|---|---|
| No. | wt % | wt % | wt % | wt % | wt % | wt % | size (nm) |
| A1 | 10 | 0 | 0 | 45 | 45 | 0 | 1100 |
| A2 | 10 | 72 | 0 | 9 | 9 | 0 | Phase |
| | | | | | | | separation |
| A3 | 10 | 0 | 72 | 9 | 9 | 0 | 258 |
| A4 | 10 | 45 | 0 | 22.5 | 22.5 | 0 | 265 |
| A5 | 10 | 0 | 45 | 22.5 | 22.5 | 0 | Phase |
| | | | | | | | separation |
| A6 | 10 | 72 | 0 | 0 | 0 | 18 | 332 |
| A7 | 10 | 0 | 72 | 0 | 0 | 18 | 811 |

The resulting solutions were stirred for 30 minutes at 80° C. Stirring was continued until THC extract was completely soluble in the oil/surfactant mix, forming a clear solution. To this clear solution was added 12 mL water with continuous stirring at 25° C. The resulting emulsion was stirred for 2 hours before particle size measurement. The particle size was measured using Dynamic Light Scattering instrument (Malvern Zetasizer Nano).

Mixing of oils and surfactants and testing in aqueous dilution studies (Table 4) yielded unexpected results in which formulations consisting of a cannabinoid in medium chain triglyceride oil and surfactants (e.g. TPGS, GELUCIRE 44/14, Polysorbate 80) were self-emulsifying with a particle size between 200-350 nm, while formulations consisting of a cannabinoid in a long chain triglyceride oil and a surfactant or surfactants form either a coarse microemulsion or aggregate (i.e. no emulsion). The percentages of surfactant and oil in Table 5 are based on the percent volume (% w/v) of surfactant and oil, excluding THC. Physical and chemical stability assays at 1 month showed no changes.

Additional formulations were prepared for in vitro and in vivo testing, as shown in Tables 5 and 6. The amount of surfactant relative to oil was increased in the formulations of Tables 5 and 6 to determine the effect on particle size and stability. The results showed a significant decrease in particle size with increasing surfactant concentration. Formulations containing oil only (no surfactant) phase separated, i.e., no particles were formed. Additional surfactants, BRIJ 97 and Solutol HS 15, were also tested with the results shown in Table 6.

TABLE 5

| | Extract | MCT | Maisine 35-1 | Sesame | GELUCIRE | TPGS | PS 80 | Particle |
|---|---|---|---|---|---|---|---|---|
| No. | wt % | wt % | (LCT) wt % | oil wt % | wt % | wt % | wt % | Size (nm) |
| A8 | 11 | 89 | 0 | 0 | 0 | 0 | 0 | — |
| A9 | 10 | 42 | 0 | 0 | 0 | 0 | 48 | 101 |
| A10 | 10 | 0 | 42 | 0 | 0 | 0 | 48 | 639 |
| A11 | 10 | 42 | 0 | 0 | 24 | 24 | 0 | 131 |
| A12 | 10 | 0 | 42 | 0 | 24 | 24 | 0 | — |
| A13 | 11 | 0 | 0 | 89 | 0 | 0 | 0 | — |

TABLE 6

| | Extract | MCT | LCT | PS 80 | BRIJ 97 | Solutol HS | Particle |
|---|---|---|---|---|---|---|---|
| No. | wt % | wt % | wt % | wt % | wt % | 15 wt % | Size (nm) |
| A14 | 10 | 48 | 0 | 42 | 0 | 0 | 101 |
| A15 | 10 | 0 | 48 | 42 | 0 | 0 | 639 |
| 16 | 11 | 24.5 | 0 | 64.5 | 0 | 0 | 26 |
| A17 | 11.5 | 0 | 24 | 64.5 | 0 | 0 | 354 |
| A18 | 9 | 47 | 0 | 0 | 44 | 0 | 223 |
| A19 | 11 | 0 | 45 | 0 | 44 | 0 | 645 |
| A20 | 9.5 | 23.5 | 0 | 0 | 67 | 0 | 353 |
| A21 | 10 | 0 | 23 | 0 | 67 | 0 | 572 |
| A22 | 11 | 47 | 0 | 0 | 0 | 42 | 126 |
| A23 | 11 | 0 | 47 | 0 | 0 | 42 | 1033 |
| A24 | 9 | 25 | 0 | 0 | 0 | 66 | 30 |
| A25 | 12 | 0 | 24 | 0 | 0 | 64 | 70 |
| A26 | 11 | 72 | 0 | 0 | 17 | 0 | 2061 |
| A27 | 10 | 0 | 73 | 0 | 17 | 0 | 1108 |
| A28 | 12 | 72 | 0 | 0 | 0 | 16 | 1794 |
| A29 | 9 | 0 | 74 | 0 | 0 | 17 | 1607 |
| A30 | 10 | 0 | 0 | 90 | 0 | 0 | 110 |
| A31 | 5 | 0 | 0 | 95 | 0 | 0 | 11 |
| A32 | 10 | 90 | 0 | 0 | 0 | 0 | Phase |
| | | | | | | | separation |

Dispersion and Dilution Behavior of Cannabinoid Compositions as a Function of Surfactant Content, Composition, and Chemistry:

Polysorbates 20, 40, 60 and 80 (or polyoxyethylene (20) sorbitan monoesters, where the lipid group is laurate, palmitate, stearate and oleate for polysorbates 20, 40, 60 and 80, respectively) and sorbitan monooleate (Span 80) were obtained from Croda Health Care or food-grade manufacturers (Modernist Pantry). For Hydrophile to Lipophile Balance (HLB) experiments, surfactant blends with varying HLB numbers between 6 and 14 were prepared by mixing Polysorbate 80 and Span 80 at different mass ratios. For higher HLB numbers from 14.9 to 16.7, pure polysorbate surfactants were used.

Cannabis extract distillate, or distillate, was obtained from New England Treatment Access (NETA, Franklin, Mass.). In-house cannabinoid potency analysis by RP-HPLC showed that the distillate was rich in Δ9-THC content (˜75%). Three other cannabinoids, cannabidiol (˜3.6%), cannabichromene (˜1.4%), tetrahydrocannabivarin (+1.3%) and cannabinol (˜0.4%) accounted for another 6.7% of the distillate mass. Five other tested major cannabinoids, cannabidivarin, cannabigerolic acid, Δ8-tetrahydrocannabinol and tetrahydrocannabinolic acid were all below quantitation limit (<0.1%).

An Agilent 1200 HPLC system equipped with a reverse-phase analytical column and a UV detector was employed for cannabinoid potency determination. The absorbance signal at 220 nm was calibrated against freshly prepared standard curve using certified reference material for 10 major cannabinoids (Cerilliant). The accuracy and limit of quantitation (LOQ) values were typically 90-110% and <0.1%, respectively.

The distillate rich in Δ9-THC content was homogenized for at least 1 hour at 75° C. Distillate-surfactant formulations with varying surfactant content of 50%, 75% and 90% (where the remainder of the formulation was the distillate) were prepared by adding the required quantity of surfactant to the distillate, followed by thorough homogenization for at least 1 hour at 75° C. in glass vials. The volume accuracy of viscous liquids was ensured by using a calibrated positive displacement pipette. The homogeneity of the formulations was assessed by visual inspection on an illuminator.

Aqueous emulsions were prepared at 1.0 or 0.1% by adding the required volume of formulation to deionized water in clean, glass vials. using a positive displacement pipette in clean, glass vials. The volume accuracy of viscous liquids was ensured by using a calibrated positive displacement pipette. After each dilution, the aqueous emulsion was vortexed for 10 seconds. Vials were visually inspected for clarity and turbidity on an illuminator and assigned a “turbidity rank” from 0 to 5 based on their apparent turbidity. Turbidity rank values of 0-5 corresponded to transparent, transparent to translucent, translucent, translucent to opaque, and opaque, respectively. Subsequently, emulsions were subjected to particle size analysis.

For particle size determination, an emulsion aliquot was loaded in UV-transparent disposable cuvettes. Time-averaged autocorrelation function data was acquired using a Malvern Instruments Zetasizer Nano DLS system at 22° C. and 90° detector angle. The manufacturer's software was used to calculate Z-average particle diameter and polydispersity values. Each sample was tested in 3 quasi-replicates and select samples were run in replicates to estimate data precision. Inter-replicate variation in Z-average particle size was typically <20%.

For this study, we identified a polysorbate-Span surfactant system as a suitable model to determine the dependence of emulsion particle size on apparent HLB number of the surfactant or surfactant blends. Here, all polysorbate surfactants had the same hydrophilic head group, while differences in HLB number was due to differences in the chain length or the degree of saturation of the lipid tail as summarized in Table 7. For polysorbates 20, 40 and 60, the lipid tail was a saturated lipid of increasing chain length, while that of polysorbate 80 was an unsaturated oleate group. Although Span 80 had the same lipid functionality as that of polysorbate 80, its HLB number was considerably lower than those of polysorbates since it is not ethoxylated. Therefore, HLB numbers between 6 and 14 were obtained by blending polysorbate 80 and Span 80 at different mass ratios, while HLB numbers 14.9, 15, 15.6, 16.7 corresponded to those of pure polysorbates 60, 80, 40 and 20, respectively.

TABLE 7
Surfactant characteristics

| Surfactant | Ester group | Lipid # | HLB number |
|---|---|---|---|
| Polysorbate 20 | Laurate | C12:0 | 16.7 |
| Polysorbate 40 | Palmitate | C16:0 | 15.6 |
| Polysorbate 60 | Stearate | C18:0 | 14.9 |
| Polysorbate 80 | Oleate | C18:1 | 15 |
| Span 80 | Oleate | C18:1 | 4.3 |

Emulsion Particle Size Vs. Surfactant HLB Number at Fixed Formulation Composition and Dilution:

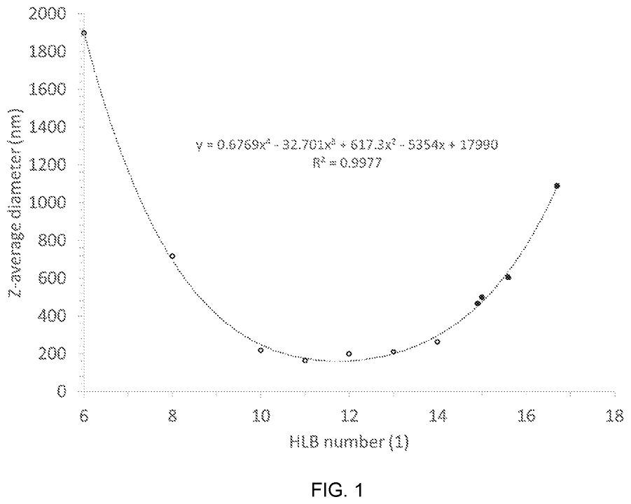

FIG. 1 shows the dependence of D, the Z-average particle diameter, on surfactant HLB number for 1.0 vol. % aqueous emulsions of formulations containing 50 vol. % surfactant. The D value showed a non-linear, parabolic dependence on the apparent HLB number of the surfactant. Starting at D≈1.9 μm for HLB=6, D values decreased gradually with increasing HLB number to a minimum of ≈180 nm at HLB=11-12. D value remained essentially constant for HLB=10-14, followed by a gradual increase in D with increasing HLB number to D≈1.1 μm at HLB=16.7. High D values for HLB <9 suggests that predominantly hydrophobic surfactants did not favor distillate microemulsions. Similarly, at a surfactant content of 50 vol. %, D values increased with increasing surfactant HLB number beyond 14. The particle size distribution indicates a preferred HLB of between about 9 to about 15, more preferably an HLB of about 10 to about 14 for distillate-surfactant formulations containing 50% surfactant. However, regardless of the surfactant HLB number, all compositions containing 50% surfactant formed turbid emulsions with high apparent turbidity with a “turbidity rank” value of 5. This suggested that despite having a Z-average diameter, D value≈200 nm, a significant population of particles exist in low surfactant content emulsions with HLB number 10-14 that are comparable in size or larger than the wavelength range of the visible light (400-700 nm). Presumably, a higher surfactant content was required to obtain clear, transparent micro-emulsions having a predominantly nanoparticle distribution.

Effect of Increasing Surfactant Content on Particle Size and its Dependence on HLB:

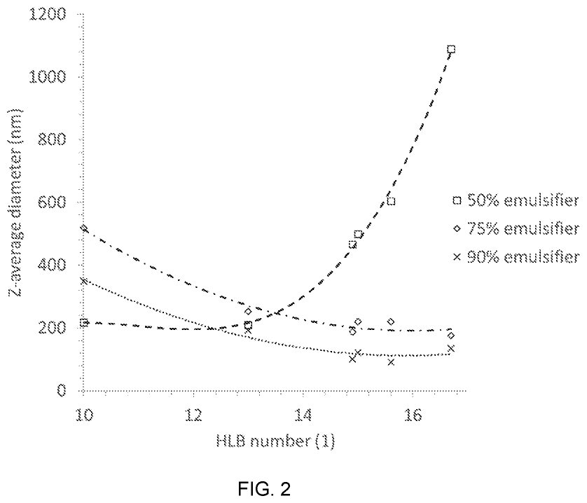

Next, the content of surfactant (HLB≥10) was increased in distillate-surfactant formulations from 50 vol. % to 75 vol. %, and to 90 vol. %, while keeping the aqueous emulsion concentration constant at 1.0 vol. %. FIG. 2 shows the dependence of D value on HLB number at different surfactant content. Surprisingly, with increasing surfactant content the dependence of particle size on HLB number was reversed and D gradually decreased with increasing HLB number for formulations containing ≥75 vol. % surfactant. The results show an overall decrease in particle size with increasing HLB number at high surfactant concentrations.

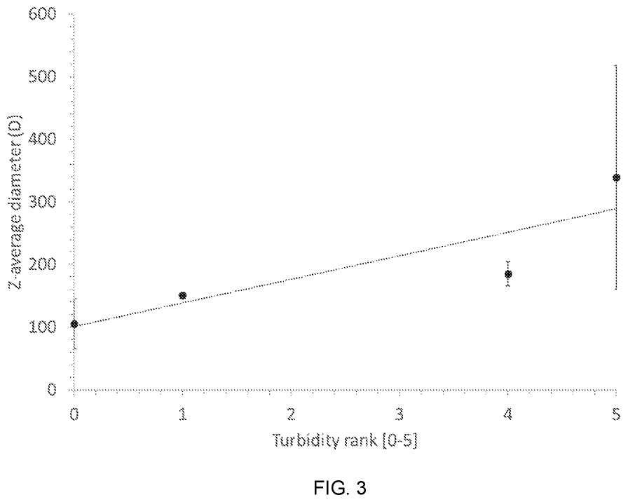

The appearance of 1.0% aqueous emulsions also changed with varying surfactant content. Formulations containing 75% surfactant formed 1.0% emulsions with a turbidity rank of 4-5, while those containing 90% surfactant formed 1.0% emulsions with a turbidity rank of 0-4. In general, apparent turbidity decreased with increasing HLB number. Also, compositions containing stearate fatty acids (polysorbate and Span 80) generally appeared more turbid. Apparent turbidity differences were most noticeable at 90% surfactant content, where turbidity rank of HLB=13 and 15 compositions were 4 and 1, respectively, while for all other, non-stearate high HLB compositions, HLB=14.9, 15.6 and 16.7, turbidity rank values were 0. As shown in FIG. 3, the apparent turbidity (turbidity rank) of the emulsions directly correlated with the Z-average particle, D data for 1.0% emulsions. Similar to low surfactant compositions, relatively high turbidity rank values for 1.0% emulsions of all 75% surfactant compositions and 90% surfactant compositions at low HLB values suggest a significant population of large particles that are able to interfere with visible light, despite their relatively low Z-average particle size measured by DLS. In contrast, the high transparency (low apparent turbidity) of 1.0% emulsions formed from 90% surfactant, high HLB compositions (HLB≥14.9) suggest that a significant population of large particles do not exist in these emulsions.

Change of Particle Size Upon Further Aqueous Dilution:

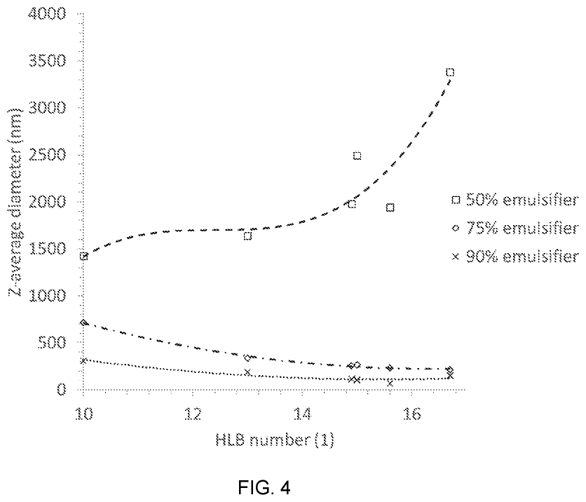

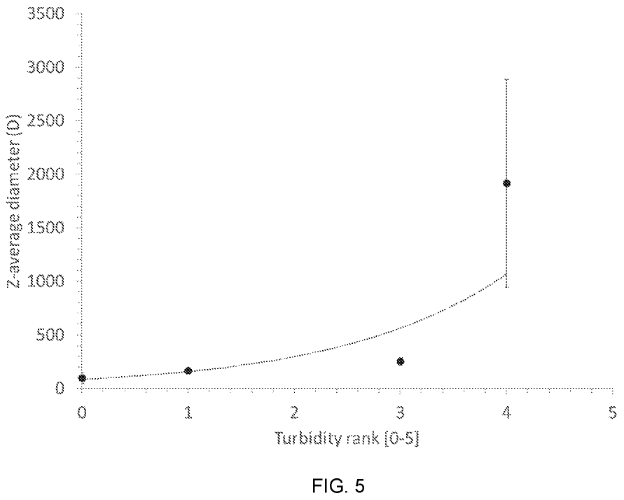

Changes in particle size upon further dilution of 1.0% aqueous emulsions were next investigated. FIG. 4 shows the dependence of D on HLB number at an aqueous emulsion concentration of 0.1%. The most pronounced change in emulsion particle size upon further dilution in water was observed for formulations with the lowest surfactant content. At 50% surfactant, D>1 μm for all 0.1% emulsions. With increasing surfactant content, the apparent change (increase) in particle size upon dilution decreased. FIG. 5 shows the direct relationship of apparent turbidity and Z-average particle size measured by DLS for 0.1% emulsions. Despite their increasing Z-average size with further dilution, the apparent turbidity of both 50% and 75% surfactant content 0.1% emulsions decreased in comparison to their 1.0% emulsions, presumably due to decreasing particle concentration. The turbidity rank of 0.1% emulsions were 4-5 and 3-4, for 50% and 75% surfactant compositions, respectively. Similar to their 1.0% emulsions, formulations containing 90% surfactant formed clear, transparent emulsions at an aqueous concentration of 0.1%, suggesting the absence of a significant population of large particles in these high surfactant content emulsions.

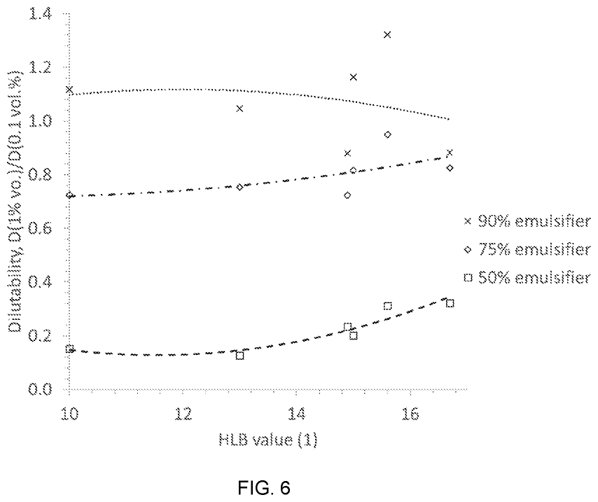

We defined a “solvent capacity” or “dilutability” parameter as the ratio of D value measured for 1.0% to D measured for 0.1% aqueous emulsions. For example, a dilutability parameter of 1.0 and 0.1 would correspond to a 0% and 900% increase in particle size upon dilution from 1.0% to 0.1%, respectively. FIG. 6 shows a comparison of dilutability curves as a function of surfactant HLB number at different surfactant content. These data suggest that regardless of the HLB number, the dilutability was low at 50% surfactant content. Increasing surfactant content to 75% significantly improved dilutability, while dilutability values were high and generally ≥0.9 for 90% surfactant content.

In Vivo Testing

The formulations of the present invention can be tested in vivo using methods well known in the art. For example, animals (e.g., beagle dogs) can be dosed with a unit dose of a cannabinoid formulation. Blood is then collected at various time points, e.g., 0.5, 1, 2, 4, 6, 8, 24, 30, 48 hours post-dose and stored (e.g., −80±10° C.) for subsequent analysis. Plasma/serum samples are then analyzed using validated methods for THC, CBD, 11-Hydroxy THC, THC-COOH. PK analysis of the concentrations of test article are determined, for example, using a non-compartmental module of WinNonlin. Individual parameters, such as, Cmax, Tmax, AUC, t1/2, Vd, and Clearance are tabulated as appropriate.

Flavoring Selection for Use as Beverage Additive:

Flavoring oils and sweetener were added to formulations A30 and A31 to determine their effect on particle size (Table 8) and their suitability as beverage additives.

TABLE 8

| | Extract | PS 80 | Lemon | Peppermint | | Particle |
|---|---|---|---|---|---|---|
| No. | wt % | wt % | Oil | Oil | Sucralose | Size |
| A30 | 10 | 90 | 0 | 0 | 0 | 110 |
| A31 | 5 | 95 | 0 | 95 | 0 | 11 |
| A33 | 9.1 | 82.3 | 2.0 | 2.0 | 4.6 | 131 |
| A34 | 4.8 | 86.2 | 2.0 | 2.1 | 4.8 | 41 |

The results for A33 and A34 showed that the addition of flavor oils to the polysorbate 80-based formulation of A30 and A31 had little impact on particle size or dissolution of the cannabinoid extract.

Additional beverage additives (Table 9) were prepared and tested.

TABLE 9

| | wt % | wt % | wt % | wt % | wt % | wt % |
|---|---|---|---|---|---|---|
| No. | Polysorbate 80 | THC-distillate | CBD | Sucralose | Peppermint | Lemon |
| BA9 | 86.9 | 0.1 | 4.5 | 4.6 | 2.0 | 1.9 |
| BA10 | 86.9 | 0.5 | 4.1 | 4.6 | 2.0 | 1.9 |

| | wt % | wt % | wt % Steam | wt % | wt % | Flavor |
|---|---|---|---|---|---|---|
| No. | Polysorbate 80 | THC-distillate | distillate | Sucralose | Flavor | description |
| BA11 | 90.0 | 5.0 | 0.0 | 5.0 | 0.0 | N/A |
| BA12 | 78.3 | 4.3 | 13.0 | 4.3 | 0.0 | N/A |
| BA13 | 85.7 | 4.8 | 0.0 | 9.5 | 0.0 | N/A |
| BA14 | 75.0 | 4.2 | 0.0 | 4.2 | 16.7 | Peppermint oil, FCC |
| BA15 | 75.0 | 4.2 | 0.0 | 4.2 | 16.7 | Lemon oil, Cold-pressed, FCC |
| BA16 | 75.0 | 4.2 | 0.0 | 4.2 | 16.7 | Artificial Lemon Flavor |
| BA17 | 75.0 | 4.2 | 0.0 | 4.2 | 16.7 | Natural Orange Flavor |
| BA18 | 75.0 | 4.2 | 0.0 | 4.2 | 16.7 | Artificial Orange Flavor |
| BA19 | 75.0 | 4.2 | 0.0 | 4.2 | 16.7 | Artificial Lime Flavor |
| BA20 | 75.0 | 4.2 | 0.0 | 4.2 | 16.7 | Dragonfruit Flavor |
| BA21 | 75.0 | 4.2 | 0.0 | 4.2 | 16.7 | Passionfruit Flavor |

| No. | Flavor Composition |
|---|---|
| BA14 | ≥50% menthol, ≥5% total esters as menthyl acetate |
| BA16 | 70-80% limonene, 7.5-10% citral, 0.1-0.5% linalool, 0.1-0.5% beta-pinene, |
| | 0.1-0.5% eucalyptol, 0.1-0.5% citronellol, 0.1-0.5% geraniol, |
| | 0.1-0.5% nerol, 0.5-1% geranyl acetate, vitamin E |
| BA17 | 80-90% limonene, 0.5-1% citral, 1-2.5% linalool, 0.1-0.5% perillaldehyde |
| BA18 | 80% medium-chain triglycerides, 10-15% limonene, 0.1-0.5% citral, |
| | 0.5-1% linalool, 0.5-1% acetaldehyde, vitamin E |
| BA19 | 72% medium-chain triglycerides, glycerin triacetate |

The artificial orange flavoring contained 80% medium-chain triglycerides, 10-15% limonene, 0.5-1% acetaldehyde, 0.5-1% linalool, 0.1-0.5% citral and vitamin E.

Enhancing the Beverage Additive Dissolution Rate by Use of Co-Solvents:

Formulations BA16, BA18, and BA19 had superior taste-masking, however, the addition of the flavoring significantly increased the dissolution time (the time to form a transparent micellar dispersion after being added to an aqueous medium). In an attempt to improve dissolution, a series of formulations (Table 10) comprising a co-solvent were made and tested. Dissolution rate was assessed visually, by adding formulations (corresponding to 10 mg cannabinoid dose) to 237 ml water at room temperature followed by brief mixing (approx. 10 sec.) using a stir bar. After mixing, samples were observed for dissolution as determined by formation of a clear, single-phase emulsion. For BA18, complete dissolution took place between 1-2 hours, while co-solvent added formulations, BA22-BA24 dissolved within 5 minutes. The ascending order of dissolution time among co-solvent added formulations was: BA22 (ethanol)≤BA23 (ethyl lactate)<BA24 (propylene glycol). At a co-solvent concentration of 24-30 wt %, ethanol (BA22) and ethyl acetate (BA23) formulations immediately formed a clear, single-phase emulsion while stirring, while dissolution of propylene glycol formulation (BA24) took up to 5 minutes. The transparency of the aqueous single-phase emulsions indicated an average particle size of 100 nm. The formulations demonstrated a rapid onset of action (about 15-25 minutes).

TABLE 10

| | wt % | wt % | wt % | wt % Artificial | wt % | |
|---|---|---|---|---|---|---|
| No. | Polysorbate 80 | THC-distillate | Sucralose | Orange Flavor | Co-solvent | Co-solvent |
| BA22 | 56.6 | 3.1 | 3.1 | 12.6 | 24.6 | Ethanol |
| BA23 | 52.9 | 2.9 | 2.9 | 11.8 | 29.5 | Ethyl lactate |
| BA24 | 52.7 | 2.9 | 2.9 | 11.7 | 29.7 | Propylene glycol |

Enhancing the Dissolution Rate with Ethanol Co-Solvent in Binary Ethanol:

### Surfactant Placebo Formulations:

Viscosity Measurements

Viscosity measurements were conducted using a microchannel viscometer (Rheosense microVISC). The viscosity of each formulation was measured in triplicate at ambient temperature.

Visual Assessment of Dissolution Rate

Dissolution rate was measured by adding each formulation to deionized water to obtain a 0.1 wt % formulation, while stirring the deionized water at 100 rpm. Dissolution time and emulsion appearance were assessed visually.

Polysorbate 80: Ethanol System:

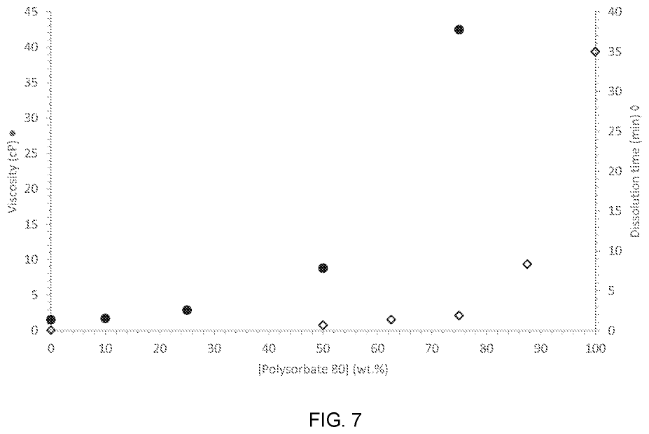

FIG. 7 shows the dependence of dissolution time and viscosity on polysorbate 80 concentration in binary ethanol: polysorbate 80 systems (0 wt. % and 100 wt. % polysorbate 80 correspond to 100 wt. % and 0 wt. % ethanol, respectively).

Both viscosity and dissolution time increased with increasing polysorbate 80 content. Therefore, increasing ethanol content led to faster dissolution in a manner that correlated well with a corresponding decrease in formulation viscosity. Preferred compositions enabling dissolution of the formulation within 2-3 minutes contained ≥25 wt. % ethanol and have a viscosity value ≤45 cP.

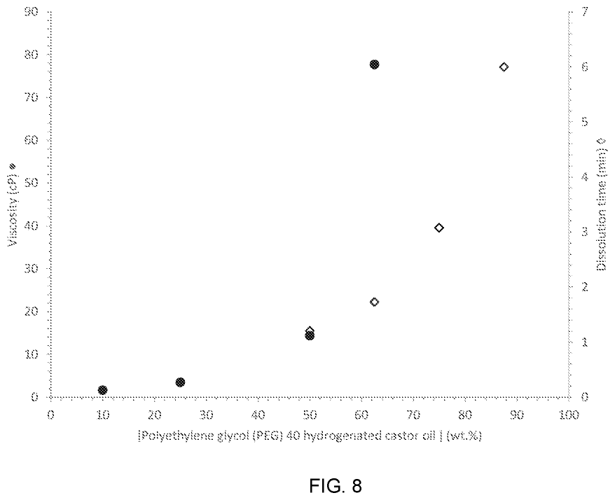

Polyethylene glycol (PEG) 40 hydrogenated castor oil: Ethanol System: FIG. 8 shows the dependence of dissolution time and viscosity on polyethylene glycol (PEG) 40 hydrogenated castor oil concentration in binary ethanol: polyethylene glycol (PEG) 40 hydrogenated castor oil systems (0 wt. % and 100 wt. % polyethylene glycol (PEG) 40 hydrogenated castor oil correspond to 100 wt. % and 0 wt. % ethanol, respectively). Both viscosity and dissolution time increased with increasing polyethylene glycol (PEG) 40 hydrogenated castor oil content. Therefore, increasing ethanol content led to faster dissolution in a manner that correlated well with a corresponding decrease in formulation viscosity. Preferred compositions enabling dissolution of the formulation within 2-3 minutes contained ≥25 wt. % ethanol. More preferred formulations enabling dissolution within 2 minutes contained ≥37.5 wt. % ethanol and had a viscosity value ≤80 cP.

These experiments suggest that for rapid dispersion formulations comprising polysorbate 80 and/or polyethylene glycol (PEG) 40 hydrogenated castor oil systems, a lower limit of ≥25 wt. % ethanol concentration is preferred.

### Dependence of Organoleptic Properties on Surfactant Chemistry:

Possible effects of surfactant selection on organoleptic properties was assessed by substituting polysorbate 80 in BA22 formulation with an equal amount of polyethylene glycol (PEG) 40 hydrogenated castor oil (Table 11). Formulations comprising polyethylene glycol (PEG) 40 hydrogenated castor oil were superior to polysorbate 80 with regards to taste. Formulations comprising polyethylene glycol (PEG) 40 hydrogenated castor oil formed clear, transparent dispersions at an aqueous concentration of 0.1%. Formulation BA25 had equivalent onset, peak time, and duration, but was superior to BA22 in taste.

TABLE 11

| | wt % | | | | |
|---|---|---|---|---|---|
| | Kolliphor | wt % | wt % | wt % | wt % |
| No. | RH40 | THC-distillate | Sucralose | Flavor | Ethanol |
| BA25 | 56.8 | 3.2 | 3.2 | 12.6 | 24.3 |

### Determination of Maximum Dispersible Concentration of BA22 and BA25 Formulations:

Dilution Experiments

Aqueous emulsions were prepared by diluting BA22 and BA25 formulations to different final THC-distillate concentrations (2, 1, 0.5, 0.25, 0.1 and 0.05 wt. % THC distillate). Dilutions were prepared using DI water at 3 different temperatures (4° C., 21° C. and 60° C.). After dilution, each vial was visually inspected. The transparency was recorded as either opaque, translucent or transparent. The formation of a transparent emulsion was attributed to a predominantly nanoparticulate size distribution.

For BA25, all emulsions (0.05-2 wt. % THC distillate) were transparent at all tested temperatures (4° C., 21° C. and 60° C.).

For BA22, at 2 lower temperatures (4° C. and 21° C.), emulsions transitioned from an opaque emulsion (at 2 wt %) to a translucent emulsion (at 0.25 wt. %) and from a translucent emulsion to a transparent emulsion (at 0.1 wt. %) with increasing dilution. For BA22, the transition from an opaque to a translucent emulsion (at 0.1 wt. %) and from a translucent to a transparent emulsion (at 0.05 wt. %) occurred at lower THC-distillate concentration compared to that for lower temperatures.

The THC-distillate concentration values at which the emulsions were transparent were attributed to a majority nanoparticulate size distribution. Correspondingly, the maximum THC-distillate concentration with majority nanoparticulate size distribution ([CN]max) values for 2 tested formulations were determined as (see Table 12):

TABLE 12
[CN]ₘₐₓ values for BA22 and BA25 formulations
at different dilution temperatures.

| | [CN]ₘₐₓ (wt. %) | | |
|---|---|---|---|
| No. | 4° C. | 21° C. | 60° C. |
| BA22 | 0.1-0.25 | 0.1-0.25 | 0.05-0.1 |
| BA25 | >2 | >2 | >2 |

The results indicate that the [CN]ₘₐₓ values were significantly higher for the polyethylene glycol (PEG) 40 hydrogenated castor oil HCO formulation (BA25) vs. polysorbate 80 formulation (BA22). Based on [CN]ₘₐₓ values, BA25 was the preferred formulation.

Estimation of Compositional Boundaries for Rapid Self-Emulsification into Micellar Dispersions:

The maximum preferred THC-distillate concentration in a rapid dispersion THC-distillate: polyethylene glycol (PEG) 40 hydrogenated castor oil: ethanol system was estimated using the following method. First, formulations at two extreme THC-distillate concentrations were prepared. A stock placebo polyethylene glycol (PEG) 40 hydrogenated castor oil: ethanol formulation (6-32-1) with minimum preferred ethanol content (25 wt. %) was used as lower (0 wt. %) extreme. A second stock formulation (6-32-2) containing THC-distillate: polyethylene glycol (PEG) 40 hydrogenated castor oil: ethanol in 1:1:1 mass ratio was used as the upper extreme. After assessing the dissolution of these 2 stock solutions, formulation iterations were prepared using these 2 stock solutions to determine the maximum THC-distillate concentration at which a transparent (nanoparticulate-rich) dispersion was obtained upon dissolution. Table 13 shows the resulting compositions and dissolution data for this system:

TABLE 13
Determination of maximum quickly dispersible THC-distillate concentration in THC-
distillate:polyethylene glycol (PEG) 40 hydrogenated castor oil:ethanol system.

| | Composition (wt. %) | | | | | |
|---|---|---|---|---|---|---|
| | | PEG-40 | | Dissolution | | |
| | | hydrogenated | | No. of | | Dissolution |
| No. | Ethanol | castor oil | THC-distillate | phases | Appearance | time (min) |
| 6-32-1 | 25 | 75 | 0 | 1 | Transparent | <1 |
| 6-32-2 | 33 | 33 | 33 | 2 | Oil + Turbid phase | N/A |
| 6-32-3 | 31 | 44 | 25 | 1 | Translucent | ~1 |
| 6-32-4 | 29 | 54 | 17 | 1 | Transparent | <2 |

Based on these results, the maximum rapid dispersion THC distillate concentration was approx. 16-24 wt. %. Thus, some rapid dispersion compositions contained approximately 0-25 wt. % THC-distillate. At around the lower ethanol limit of 25 wt. % (29 and 31 wt. % for 6-32-3 and 6-32-4, respectively), a minimum of 43.5-54 wt. % polyethylene glycol (PEG) 40 hydrogenated castor oil was required to obtain a transparent dispersion. Therefore, some ternary THC-distillate: polyethylene glycol (PEG) 40 hydrogenated castor oil: ethanol rapid dispersion compositions comprised approximately ≥25 wt. % ethanol and ≤25 wt. % THC-distillate.

TABLE 14

| | Composition (wt. %) | | | | | |
|---|---|---|---|---|---|---|
| | | PEG-40 | | | Artificial | |
| | | hydrogenated | | | Orange | Emulsion |
| No. | THC-distillate | castor oil | Ethanol | Sucralose | Flavor* | Appearance** |
| 6-17-4 | 7.6 | 22.8 | 50.6 | 3.8 | 15.2 | TL |
| 6-17-7 | 7.6 | 26.0 | 47.4 | 3.8 | 15.2 | TL |
| 6-17-6 | 7.6 | 29.2 | 44.2 | 3.8 | 15.2 | TP |
| 6-17-5 | 7.6 | 35.6 | 37.8 | 3.8 | 15.2 | TP |
| 6-17-3 | 7.6 | 48.4 | 25.0 | 3.8 | 15.2 | TP |
| 6-17-9 | 5.0 | 15.0 | 55.0 | 5.0 | 20.0 | O |
| 6-17-10 | 5.0 | 30.0 | 40.0 | 5.0 | 20.0 | TL |
| 6-17-12 | 5.0 | 33.8 | 36.3 | 5.0 | 20.0 | TL |
| 6-17-11 | 5.0 | 37.5 | 32.5 | 5.0 | 20.0 | TP |
| 6-17-8 | 5.0 | 45.0 | 25.0 | 5.0 | 20.0 | TP |

*The artificial orange flavoring contained 80% medium-chain triglycerides, 10-15% limonene, 0.5-1% acetaldehyde, 0.5-1% linalool, 0.1-0.5% citral and vitamin E.
**For emulsion appearance, PS: Phase separation; O: Opaque, TL: Translucent, TP: Transparent.

TABLE 15

| | Composition (wt. %) | | | | | |
|---|---|---|---|---|---|---|
| | | PEG-40 | | | Artificial | |
| | | hydrogenated | | | Orange | Emulsion |
| No. | THC-distillate | castor oil | Ethanol | Sucralose | Flavor* | Appearance** |
| BA30 | 5.0 | 50.0 | 30.0 | 5.0 | 10.0 | TP |
| BA31 | 7.6 | 34.2 | 35.4 | 7.6 | 15.2 | TP |

*The artificial orange flavoring contained ≥25 wt. % d-limonene and 0.1-1 wt. % citral.
**For emulsion appearance, PS: Phase separation; O: Opaque, TL: Translucent, TP: Transparent.

Some beverage additive compositions listed in Tables 14 and 15 rapidly self-emulsified into micellar dispersions with a particle size of less than 50 nm when added to water or another beverage of choice. Some compositions contained ≤25 wt. % THC-distillate, ≥25 wt. % ethanol, ≤25 wt. % artificial orange flavor and ≤10 wt. % sucralose.

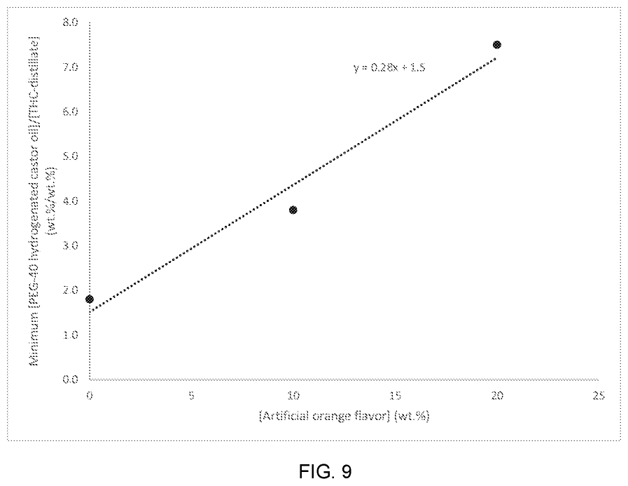

FIG. 9 shows the minimum amount of PEG-40 hydrogenated castor oil in some compositions plotted as the minimum mass ratio of PEG-40 hydrogenated castor oil to THC-distillate versus the concentration of artificial orange flavor (artificial orange flavoring contained 80% medium-chain triglycerides, 10-15% limonene, 0.5-1% acetaldehyde, 0.5-1% linalool, 0.1-0.5% citral and vitamin E).

Some compositions contained a minimum amount of PEG-40 hydrogenated castor oil that satisfies equation [1]. Equation [1] describes the minimum PEG-40 hydrogenated castor oil (PEG-40 HCO) to THC-distillate mass ratio in some compositions as a function of the concentration of the artificial orange flavor (artificial orange flavoring contained 80% medium-chain triglycerides, 10-15% limonene, 0.5-1% acetaldehyde, 0.5-1% linalool, 0.1-0.5% citral and vitamin E):

[PEG-40 HCO/[THC-distillate]≥0.28*[Artificial orange flavor*]+1.5 Equation [1]

Compositions Comprising a Cannabinoid and Ethyl Pyruvate:

CBD extract, Ethyl Pyruvate (EP), and Polysorbate 80 (PS 80) were mixed in a ratio as shown in Table 16 in 20 mL scintillation vials.

The resulting solutions were stirred for 30 minutes at 80° C. Stirring was continued until CBD extract was completely soluble in the EP and PS 80 mix, forming a clear solution. This clear solution was used for dissolution studies in water by adding 45 microliters in 12 mL water (0.375%) with continuous stirring at 25° C. The resulting emulsion was stirred for 20 hours before particle size measurement. The particle size was measured using Dynamic Light Scattering instrument (Malvern Zetasizer Nano).

In this study, all CBD, EP and PS 80 ratios demonstrated high solubility. To determine how different ratios of EP and PS 80 influence the self-emulsifying properties of the mixture, dilution studies in water were performed.

TABLE 16

| | | Ethyl | | |
|---|---|---|---|---|
| | CBD | Pyruvate | PS80 | Particle |
| No. | wt % | wt % | wt % | Size nm |
| EP1 | 5 | 10 | 85 | <100 |
| EP2 | 5 | 20 | 75 | 100-500 |
| EP3 | 5 | 30 | 65 | 100-500 |
| EP4 | 5 | 40 | 55 | >500 |
| EP5 | 5 | 50 | 45 | >500 |

The results showed formulations all formed nano- or microemulsions. Formulations containing greater than 65% PS 80 produced emulsions with average particles sizes below 500 nm and formulations containing less than 65% PS 80 produced emulsions with average particle sizes above 500 nm.

Based on the results presented in Table 16. THC extract, CBD, EP, terpenes, and PS 80 were mixed in a ratio as shown in Table 17.

TABLE 17

| | No. | | | | |
|---|---|---|---|---|---|
| Composition (wt %) | EP22 | EP23 | EP24 | EP25 | EP26 |
| THC-distillate wt % | 0.52 | 0.54 | 0.51 | 0.53 | 0.1 |
| CBD wt % | 6.53 | 6.7 | 6.4 | 6.56 | 4.9 |
| Ethyl Pyruvate wt % | 29.14 | 29.89 | 9.85 | 10.1 | 1.19 |
| Beta-caryophyllene wt % | 0.4 | 0.41 | 0.39 | 0.4 | 0.333 |
| Linalool wt % | 0.4 | 0.41 | 0.39 | 0.4 | 0.047 |
| Limonene wt % | 0.4 | 0.41 | 0.39 | 0.4 | 0.736 |
| Alpha-pinene wt % | 0.4 | 0.41 | 0.39 | 0.4 | 1.281 |
| Eucalyptol wt % | 0.4 | 0.41 | 0.39 | 0.4 | 0.003 |
| Polysorbate 80 wt % | 59.28 | 60.81 | 78.8 | 80.79 | 86.42 |
| Sucralose wt % | 2.51 | 0 | 2.46 | 0 | 5 |

Edibles—Gummies:

Table 18 lists the amounts of ingredients for different gummy batch sizes. Additional batch sizes can be scaled accordingly.

TABLE 18

| | Amount of Gummy Base Ingredient Per Batch Size | | | | | | | |
|---|---|---|---|---|---|---|---|---|
| | 1X | | 2X | | 10X | | 20X | |
| Gelatin | 65 | g | 130 | g | 650 | g | 1300 | g |
| (280 | | | | | | | | |
| bloom) | | | | | | | | |
| Water | 165 | g | 330 | g | 1650 | g | 3300 | g |
| Sugar | 225 | g | 450 | g | 2250 | g | 4500 | g |
| Corn | 245 | g | 490 | g | 2450 | g | 4900 | g |
| Syrup | | | | | | | | |
| Xylitol | 26 | g | 52 | g | 260 | g | 520 | g |
| Citric | 45 | g | 90 | g | 450 | g | 900 | g |
| Acid | | | | | | | | |
| Flavor | 15 | g | 30 | g | 150 | g | 300 | g |
| Color | 1 | Drop | 2 | Drops | 10 | Drops | 20 | Drops |

Flavors (colors) used were as follows: coconut (white), blueberry (blue), strawberry-melon (green; flavor ½ and ½), watermelon (pink: use ½ number of drops of red), blood orange (red and orange equal parts), mango (light orange: use ½ number of drops of orange).
1. Ingredients are scaled to the desired size. Gelatin and water are combined and mixed well. The mixture will immediately begin to bloom.
2. Sugar, xylitol and corn syrup are combined in a pot and heated on a stove until it reaches 250° F.
3. Bloomed gelatin is added to the sugar mixture in semi-small chunks and mix well with a spatula until all gelatin melts. Gelatin mixture is weighed and amount of cannabinoid formulation required for desired dose is calculated.
4. Color, flavor, cannabinoid formulation, and citric acid are added to the gelatin mixture. The cannabinoid formulation is a cannabinoid composition of the present invention. For example, the cannabinoid formulation may consist of cannabinoid extract dissolved in MCT (total percent between 10-80 w/v) and polysorbate 80 (total percent between 10-90 w/v). The ingredients are mixed well with a mixer and poured into a funnel. Foam is allowed to come to the top (5 minutes) before pouring.
5. The mixture is poured into square pans sprayed with a non-stick spray. Foam is not allowed to pour into pans. The funnel is topped off as needed with the remaining gummy mixture.
6. Trays are transferred to a rolling rack and allowed to set up slightly before moving to refrigerator.
7. Gummies are cut into cubes. Each gummy cube typically contains a cannabinoid dose ranging from 1-10 mg.

Hard Gelatin Capsules

Unsealed hard gelatin capsules containing formulations comprising a large amount of polysorbate 80 were found to be prone to leakage. Attempts were made to prevent leakage of these polysorbate 80-rich liquid formulations during storage of capsules, without having to add any new process steps, such as capsule sealing or banding. Formulation were made that substituted a portion of the polysorbate 80 with TPGS. Formulations with a 1:0 and 3:0 ratio of polysorbate 80:TPGS leaked at ambient temperature. A formulation with a ratio of 1:1 polysorbate 80:TPGS, however, did not leak under the same conditions.

Clinical Observational Study

Observational studies including 23 subjects were conducted to compare the psychoactive effects of formulations A30 (90% Polysorbate 80 and 10% THC-distillate), A32 (90% MCT oil and 10% THC-distillate) and A34 (86.2% Polysorbate 80, 4.8% THC-distillate, 4.8% Sucralose, 2.0% Lemon oil and 2.1% Peppermint oil). A30 and A32 were supplied as capsules, while A34 was supplied as a beverage additive. The protocol was reviewed and approved by an independent ethics committee, and all subjects provided written informed consent. Subjects were recruited from two Medical Marijuana (MM) dispensaries in the Greater Boston Area. Subjects were asked to complete follow-up surveys (e.g., MM use behavior and effects) after each dispensary visit. All self-report data were collected via secure online research portal and identified only by the subject's unique ID number.

Effect:

A34 and A30 provided a more intense effect than A32. Specifically, subjects experienced a 124% greater peak effect for A34 versus A32 and 60% greater peak effect for A30 versus A32. The effect of A30 was also less variable than that of A32, with 83% lower interquartile range with A30.

Onset Time:

Subjects reported significantly faster onset of the effects of A30 than that of A32 (a=0.016). The mean onset of effects was within 31-45 minutes for A30, while that of A32 was within 46-66 minutes. For A34, the onset time of effects was significantly faster, and consistently 15-20 minutes.

Peak Time:

Similar to onset time, peak times of the effects of A34 and A30 were also shorter than that of A32. On average, peak effects were observed within 80-90 for A32, within 60 minutes for A30, and within 45 minutes for A34.

Duration:

The duration of effect that subjects experienced for A30 and A34 was similar to that of A32 but less variable, with 60% lower standard deviation.

1. A composition comprising:
(a) at least one cannabinoid or cannabinoid extract;
(b) at least one surfactant; and
(c) at least one co-solvent, wherein the at least one co-solvent is ethanol;
wherein the composition comprises no exogenously added fatty acid, monoglyceride, diglyceride, or triglyceride, or
wherein the only exogenously added fatty acid, monoglyceride, diglyceride, triglyceride, or combination thereof is a flavoring oil.

2-239. (canceled)

240. The composition of claim 1, wherein the surfactant is polysorbate 80.

241. The composition of claim 1, further comprising an antioxidant.

242. The composition of claim 241, wherein the antioxidant is selected from the group consisting of ascorbyl palmitate, butylated hydroxy anisole, butylated hydroxy toluene, propyl gallate, α-tocopherol, γ-tocopherol, and mixed tocopherols.

243. The composition of claim 242, wherein the antioxidant is present in an amount from about 0.01% w/v to about 0.1% w/v.

244. The composition of claim 1, wherein the surfactant is present in an amount of at least 60 wt %.

245. The composition of claim 244, wherein the surfactant is present in an amount of at least about 65 wt %.

246. The composition of claim 1, wherein the ethanol is present in an amount of at least about 25 wt %.

247. The composition of claim 246, wherein the ethanol is present in an amount between 25 and 35 wt %.

248. The composition of claim 247, wherein the ethanol is present in an amount between 25 and 30 wt %.

249. The composition of claim 1, wherein the cannabinoid or cannabinoid extract is present in an amount of about 2.5 to about 5 wt %.

250. The composition of claim 1, wherein the cannabinoid or cannabinoid extract is present in an amount of at least about 5 wt %.

251. The composition of claim 1, wherein the at least one cannabinoid or cannabinoid extract comprises tetrahydrocannabinol, Δ9-tetrahydrocannabinol (THC), Δ8-tetrahydrocannabinol, a cannabis extract, tetrahydrocannabinolic acid (THCA), cannabidiolic Acid (CBDA), Δ8-tetrahydrocannabinol-DMH, Δ9-tetrahydrocannabinol propyl analogue (THCV), 11-hydroxy-tetrahydrocannabinol, 11-nor-9-carboxy-tetrahydrocannabinol, 5′-azido-Δ8-tetrahydrocannabinol, AMG-1, AMG-3, AM411, AM708, AM836, AM855, AM919, AM926, AM938, cannabidiol (CBD), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabidiol propyl analogue (CBDV), cannabinol (CBN), cannabichromene (CBC), cannabichromene propyl analogue, cannabigerol (CBG), cannabicyclol (CBL), cannabielsoin (CBE), cannabinodiol (CBDL), and cannabitriol (CBTL), CP 47497, CP 55940, CP 55244, CP 50556, CT-3 or IP-751 (ajulemic acid), dimethylheptyl HHC, HU-210, HU-211, HU-308, WIN 55212-2, desacetyl-L-nantradol, dexanabinol, JWH-051, JWH-133, levonantradol, L-759633, nabilone, O-1184, cannabicyclohexanol (CP-47,497 C8 homolog), 10-hydroxycannabidiol, 1′,2′,3′,4′,5′-pentanorcannabinol-3-carboxylic acid, l′-hydroxycannabinol, 11-hydroxycannabinol, 9-carboxy-11-norcannabinol, 1′-oxocannabinol, 11-nor-Δ8-THC-9-carboxylic acid, T-carboxy-3′,4′,5′-trinor-Δ9-THC, 5′-carboxy-Δ9-THC, 9-carboxy-11-nor-Δ9-THC, 9-carboxy-11-nor-Δ8-THC, [(6aR,10aR)-3-[(1S,2R)-1,2-dimethylheptyl]-6a,7,10,10a-tetrahydro-6, 6,9-trimethyl-6H-dibenzo[b,d]pyran-1-ol], 9-carboxy-11-nor-(2 or 4)-chloro-Δ8-THC, 8α-11-dihydroxy-Δ9-THC, 8β-11-Dihydroxy-Δ9-THC, 5′-Dimethylamino-Δ8-THC, 11-hydroxy-Δ9-THC, 1′-hydroxy-Δ9-THC (Isomer B), 11-hydroxy-Δ8-THC, 2′-hydroxy-Δ9-THC, 3′-hydroxy-Δ9-THC, 4′-hydroxy-Δ9-THC, 5′-hydroxy-Δ9-THC, 8α-hydroxy-Δ9-THC, 8β-hydroxy-Δ9-THC, 5′-methylamino-Δ8-THC, 5′-N-methyl-N-4-(7-nitrobenzofurazano)amino-Δ8-THC, (−)-trans-Δ8-THC, 5′-trimethylammonium-Δ8-THC phenolate, 5′-Trimethylammonium-11-hydroxy-Δ8-THC phenolate, or a mixture thereof.

252. The composition of claim 251, wherein the at least one cannabinoid or cannabinoid extract comprises THC, CBD, THCA or CBDA, or a combination thereof.

253. The composition of claim 1, wherein said composition is a non-aqueous composition.

254. The composition of claim 1, wherein the composition is a beverage additive.

255. A composition comprising an aqueous emulsion or a micellar dispersion of the self-emulsifying composition of claim 1 in an aqueous solvent.

256. The composition of claim 255, wherein particles of the aqueous emulsion or the micellar dispersion have an average particle size less than about 100 nm.

257. The composition of claim 256, wherein the average particle size is less than about 50 nm.

258. The composition of claim 257, wherein the average particle size is between about 10 and about 50 nm.

259. The composition of claim 1, wherein the composition is a solid or semi-solid composition.

260. The composition of claim 1, wherein the composition is an oral dosage form.

261. The composition of claim 260, wherein the oral dosage form is a liquid.

262. The composition of claim 260, wherein the oral dosage form is a capsule.

263. A unit dosage form of the composition of claim 255, wherein the unit dose contains between about 0.5 and about 2.5 mg of total cannabinoid(s).
